(19) United States
(12) Patent Application Publication
(10) Pub. No.: US 2021/0117985 A1
(43) Pub. Date: Apr. 22, 2021

(54) ANALYTICS ENGINE FOR DETECTING MEDICAL FRAUD, WASTE, AND ABUSE

(71) Applicant: Alivia Capital LLC, Woburn, MA (US)

(72) Inventor: Kleber Gallardo, Woburn, MA (US)

(21) Appl. No.: 17/132,644

(22) Filed: Dec. 23, 2020

Related U.S. Application Data
(63) Continuation of application No. 15/462,312, filed on Mar. 17, 2017, now abandoned.
(60) Provisional application No. 62/310,176, filed on Mar. 18, 2016.

(51) Int. Cl. G06Q 30/00; G06N 20/00; G06Q 40/08; G06Q 10/06; G06Q 50/22; G06N 3/04; G06N 5/02; G06N 3/08; G06F 30/20; G06N 20/20; H04L 12/24; H04L 29/08

(52) U.S. Cl. CPC G06Q 30/0185; H04L 67/2842; G06Q 40/08; G06Q 10/063; G06Q 50/22; G06N 3/0454; G06N 5/025; G06N 3/08; G06F 30/20; G06N 20/20; H04L 41/0809; H04L 67/02; H04L 67/12; H04L 67/2804; G06N 20/00

(57) ABSTRACT

Exemplary embodiments relate to a Health Care Fraud Waste and Abuse predictive analytics projects sharing network where analytic models can be shared and used directly with minimum changes. The shared/passed Models and Rules on the network are directly applied to datasets from different customers by mapping and creating useful results electronically within a healthcare claims space. A drag-and-drop graphical user interface simplifies the creation of models by associating one or more data sources with one or more pre-defined plug-and-play application graphically.

### CROSS-REFERENCE TO RELATED APPLICATION(S)

This patent application is a continuation of, and therefore claims priority from, U.S. patent application Ser. No. 15/462,312 entitled ANALYTICS ENGINE FOR DETECTING MEDICAL FRAUD, WASTE, AND ABUSE filed Mar. 17, 2017, which claims the benefit of U.S. Provisional Patent Application No. 62/310,176 filed Mar. 18, 2016. Each of these patent applications is hereby incorporated herein by reference in its entirety.

### FIELD OF THE INVENTION

The invention generally relates to data analytics and, more particularly, the invention relates to visualizations of data analytics.

### BACKGROUND OF THE INVENTION

U.S. healthcare expenditure in 2014 was roughly 3.8 trillion. The Centers for Medicare and Medicaid Services (CMS), the federal agency that administers Medicare, estimates roughly $60 billion, or 10 percent, of Medicare's total budget was lost to fraud, waste, and abuse. In fiscal year 2013, the government only recovered about $4.3 billion dollars.

### SUMMARY OF VARIOUS EMBODIMENTS

In accordance with one embodiment of the invention, a healthcare fraud detection system comprises a user interface, a core processing system coupled to the user interface and also coupled to a database storage, and a data input providing healthcare data, the data input being user selectable from at least one data source, the data input being coupled to the core processing system. The core processing system comprises a set of stored pre-defined plug-and-play applications configured to manipulate the data, and the core processing system is configured to permit, via the user interface, drag-and-drop selection and interconnection of at least one data source and at least one pre-defined plug-and-play application by a user to produce a healthcare fraud detection model and to display, via the user interface, fraud analytics data produced by the healthcare fraud detection model.

In various alternative embodiments, the user interface may be a web-browser interface. The core processing system may display the least one data source and the at least one pre-defined plug-and-play application as interconnected icons on the user interface.

The core processing system may include a deep learning engine, such as a machine learning engine, configured to process the data. The deep learning engine may be configured to automatically determine a set of performance metrics and a plurality of algorithms to use for the at least one data source and create therefrom an ensemble of models, where each component in the ensemble is a deep learning model focusing on a specific type of fraud. The deep learning engine may be configured to detect medical claim fraud in real time, or substantially in real time, from a stream of medical claims.

In other embodiments, graphs and/or dashboards may be reusable artifacts that are part of a template that can be integrated with data sources, filters and models to build a complete template. The core processing system may allow the user to alter the display of the fraud analytics data. The core processing system may allow sharing of the healthcare fraud detection model over a network. The set of stored pre-defined plug-and-play applications may include an analyzer operator, which may be configured to extract meta-data from the at least one data source, perform data cleansing on a set of user-specified fields, select a set of default metrics for use in comparing performance of a plurality of fraud detection models, select a set of operators to be applied to the data, format the data for each selected operator, execute the selected operators, and determine a best model from the plurality of models based on the execution of the selected operators. The set of stored pre-defined plug-and-play applications additionally or alternatively may include at least one filter operator, at least one fraud detection operator, and/or at least one visualization operator. The core processing system may allow the user to associate the at least one data source and the healthcare fraud detection model as a project, which may be shared over a network. The core processing system may allow the user to export results from the healthcare fraud detection model to CSV.

In certain embodiments, the healthcare fraud detection system additionally may include a distributed in-memory cache coupled to the core processing system. The core processing system may run on a distributed computing cluster and may utilize a distributed file system.

Additional embodiments may be disclosed and claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1 provides a summary of features provided by an exemplary embodiment referred to as Absolute Insight.

FIG. 2 is a schematic block diagram of the distributed architecture of Absolute Insight, in accordance with one exemplary embodiment.

FIGS. 3-11 show various interactions between components in the Absolute Insight distributed architecture, in accordance with one exemplary embodiment.

FIG. 12 is a sample annotated screen that shows various operations that can be performed by a user, in accordance with one exemplary embodiment.

FIG. 13 shows a sample annotated screen for viewing the items currently residing in a particular project or information regarding a particular project, in accordance with one exemplary embodiment.

FIG. 14 shows a sample annotated screen for creating a new project, in accordance with one exemplary embodiment.

FIG. 15 shows a sample annotated screen with two participants added to a project, in accordance with one exemplary embodiment.

FIG. 16 shows a sample annotated Data Repository screen such as might be presented when the user selects the “Data Repository” tab, in accordance with one exemplary embodiment.

FIG. 17 shows a sample annotated screen from which the user can take a quick peak into the actual data in order to understand what is inside the data source, in accordance with one exemplary embodiment.

FIG. 18 shows a sample annotated screen such as might be displayed when the user selects the “Data Cleansing” tab, in accordance with one exemplary embodiment.

FIG. 19 is a sample annotated screen with a popup window allowing the user to choose if and where a particular rule is applied, in accordance with one exemplary embodiment.

FIG. 20 is a sample annotated screen such as might be displayed when the user selects the “Query Builder” tab, in accordance with one exemplary embodiment.

FIG. 21 is a sample annotated screen showing a portion of the sample screen of

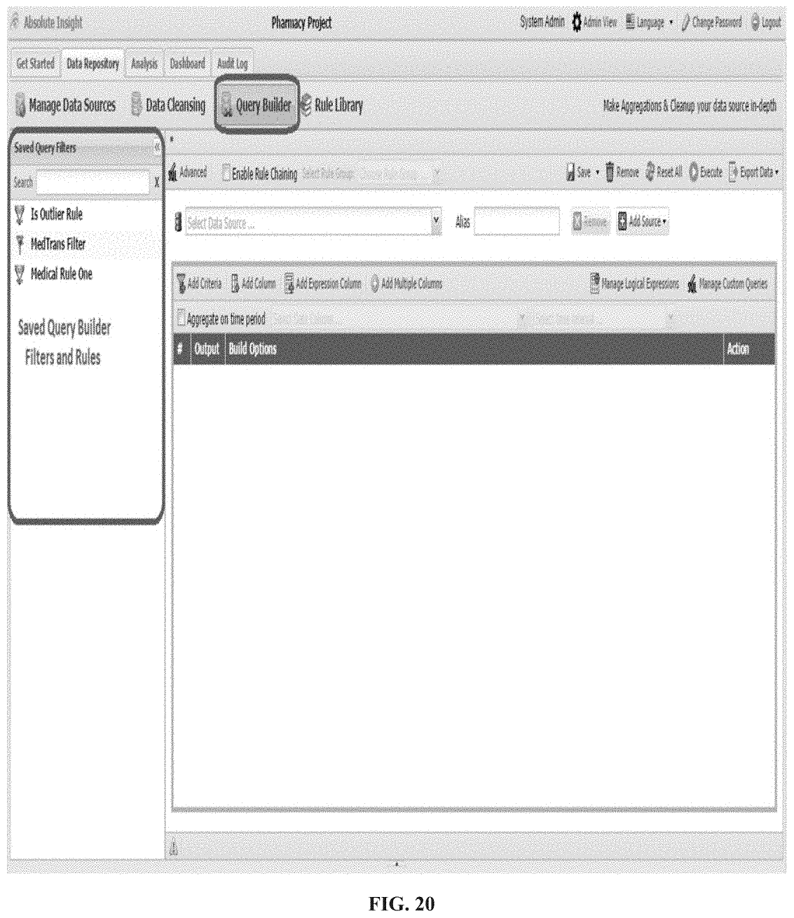

FIG. 20 highlighting a first set of controls.

FIG. 22 is a sample annotated screen showing a portion of the sample screen of

FIG. 20 highlighting a second set of controls.

FIG. 23 is a sample annotated screen showing a popup box such as when the “Add Multiple Columns” button is selected in FIG. 22, in accordance with one exemplary embodiment.

FIG. 24 shows a portion of a sample annotated screen displaying two rule groups, in accordance with one exemplary embodiment.

FIG. 25 shows an expanded version of the sample screen of FIG. 24 with further annotations.

FIG. 26 shows a sample annotated screen from which the user can create a set of queries in the query builder, in accordance with one exemplary embodiment.

FIG. 27 shows a sample annotated screen from which the user can save a query, in accordance with one exemplary embodiment.

FIG. 28 shows a sample annotated screen where a query used as a rule in some rule group is shown with an icon different from the icons of a standard query, in accordance with one exemplary embodiment.

FIG. 29 shows a sample annotated screen that allows the user to configure a rule, in accordance with one exemplary embodiment.

FIG. 30 shows a sample annotated screen for saving a Query Builder item as a rule snippet, in accordance with one exemplary embodiment.

FIG. 31 shows a sample annotated screen where a rule snippet is available but cannot be executed, in accordance with one exemplary embodiment.

FIG. 32 shows a sample annotated screen where the snippet rule can now be used in Rule Chaining, in accordance with one exemplary embodiment. for example, as depicted in the sample annotated screen shown in FIG. 32.

FIG. 33 shows a sample annotated screen where a rule snippet is used by just referring to the snippet, in accordance with one exemplary embodiment.

FIG. 34 shows a sample screen for saving a new Chained Rule as a normal Rule, in accordance with one exemplary embodiment.

FIG. 35 shows a sample annotated screen where the user can execute a rule directly inside Query Builder to see results immediately, in accordance with one exemplary embodiment.

FIG. 36 shows a sample annotated screen where the user can execute a new Chained Rule in the Rule Library and generate results, in accordance with one exemplary embodiment.

FIG. 37 shows a sample annotated screen with a visualization integrated into a dashboard, in accordance with one exemplary embodiment.

FIG. 38 is a sample screen showing an analysis of the a data source with the results sorted by rank, in accordance with one exemplary embodiment.

FIG. 39 is a sample screen showing an example of a model created using drag-and-drop operations provided by the graphical user interface (GUI) of the application, in accordance with one exemplary embodiment.

FIG. 40 shows a sample annotated screen showing an example of model creation using drag-and-drop operations provided by the graphical user interface (GUI), in accordance with one exemplary embodiment.

FIG. 41 shows a sample annotated screen where the user is automatically taken to a “Dashboard “View” following completion of model execution, in accordance with one exemplary embodiment.

FIG. 42 is a sample annotated screen showing various types of operators, in accordance with one exemplary embodiment.

FIG. 43 shows a sample annotated screen showing all the models that are saved by the user from the modeling canvas, in accordance with one exemplary embodiment.

FIG. 44 shows a sample annotated screen allowing the user to plot any chart, in accordance with one exemplary embodiment.

FIG. 45 is a sample annotated screen that highlights that a potentially high-risk doctor has been identified, in accordance with one exemplary embodiment.

FIG. 46 shows a sample annotated screen demonstrating how a potentially high-risk provider can be found by simply plotting TOT_AMT_PAID against PHYSICIAN_NAME using a pie chart, in accordance with one exemplary embodiment.

FIG. 47 shows a sample screen where the total amount that has been paid to a physician in shown, in accordance with one exemplary embodiment.

FIG. 48 shows a sample annotated screen for filtering data, in accordance with one exemplary embodiment.

FIG. 49 shows a sample annotated screen providing an example of a bar chart with gradient color, in accordance with one exemplary embodiment.

FIG. 50 is a sample annotated screen showing a scatter plot using the Detail, Color and Size input slots, in accordance with one exemplary embodiment.

FIG. 51 is a sample screen of a Tree Map showing physicians grouped by their geographical location and locations with the highest propability of containing outliers, in accordance with one exemplary embodiment.

FIG. 52 is a sample annotated screen showing a back button allowing the user to drill back up, in accordance with one exemplary embodiment.

FIG. 53 shows a sample annotated screen where user can select any Descriptor in Columns and drag and drop any measure against it in Rows, in accordance with one exemplary embodiment.

FIG. 54 is a sample annotated screen showing a grouped bar chart with more than one set of values that the user wants to see side by side, in accordance with one exemplary embodiment.

FIG. 55 shows a sample annotated screen of an area chart corresponding to the grouped bar chart of FIG. 54, in accordance with one exemplary embodiment.

FIG. 56 shows a sample annotated screen with a selected chart highlighted in the charting palette and also showing the required ingredients to make that chart, in accordance with one exemplary embodiment.

FIG. 57 shows a sample annotated screen for drawing a table, in accordance with one exemplary embodiment.

FIG. 58 shows a sample annotated screen showing a “Choose Columns” pop-up screen to allow the user to select the columns to use for the table, in accordance with one exemplary embodiment.

FIG. 59 shows a sample annotated screen with resulting information from the selections in FIG. 58, in accordance with one exemplary embodiment.

FIG. 60 shows a sample annotated screen for adding a grid or a chart to a dashboard, in accordance with one exemplary embodiment.

FIG. 61 shows a sample screen with Model Execution History information, in accordance with one exemplary embodiment.

FIG. 62 shows a sample screen with Rule Execution Results information, in accordance with one exemplary embodiment.

FIG. 63 is a sample annotated screen with an audit log, in accordance with one exemplary embodiment.

FIG. 64 shows a sample annotated screen providing the user with an option to export logs into a csv formatted document, in accordance with one exemplary embodiment.

FIG. 65 is a sample annotated screen for exporting a Data Source into CSV from the Manage Data Sources tab, in accordance with one exemplary embodiment.

FIG. 66 is a sample annotated screen for exporting cleansing filter results data into CSV from the Data Cleansing tab, in accordance with one exemplary embodiment.

FIG. 67 is a sample annotated screen for exporting query filter results data into CSV from the Query Builder tab, in accordance with one exemplary embodiment.

FIG. 68 is a sample annotated screen for exporting Dashboard results data into CSV, in accordance with one exemplary embodiment.

FIG. 69 shows a sample annotated screen with Organization access permissions that can be viewed and updated, in accordance with one exemplary embodiment.

FIG. 70 shows a sample annotated screen for creating a new Organization with access permissions, in accordance with one exemplary embodiment.

FIG. 71 shows a sample annotated screen with Region access permissions that can be viewed and updated, in accordance with one exemplary embodiment.

FIG. 72 shows a sample annotated screen for creating a new Region with access permissions, in accordance with one exemplary embodiment.

FIG. 73 shows a sample annotated screen with User Groups access permissions that can be viewed and updated, in accordance with one exemplary embodiment.

FIG. 74 shows a sample annotated screen for creating a new User Group with access permissions, in accordance with one exemplary embodiment.

FIG. 75 shows a sample annotated screen with User access permissions that can be viewed and updated, in accordance with one exemplary embodiment.

FIG. 76 shows a sample annotated screen for importing users into the application, in accordance with one exemplary embodiment.

FIG. 77 is a schematic block diagram of an Analyzer Operator, in accordance with one exemplary embodiment.

FIG. 78 shows various steps for performing deep learning, in accordance with one exemplary embodiment.

### DESCRIPTION OF ILLUSTRATIVE EMBODIMENTS

Exemplary embodiments relate to a Health Care Fraud Waste and Abuse predictive analytics projects sharing network where analytic models can be shared and used directly with minimum changes. The shared/passed Models and Rules on the network are directly applied to datasets from different customers by mapping and creating useful results electronically within a healthcare claims space.

In illustrative embodiments, a browser based software package provides quick visualization of data analytics related to the healthcare industry, primarily for detecting potential fraud, waste, abuse, or possibly other types of anomalies (referred to for convenience generically herein as “fraud”). Users are able to connect to multiple data sources, manipulate the data and apply predictive templates and analyze results. Details of illustrative embodiments are discussed below with reference to a product called Absolute Insight from Alivia Technology of Woburn, Mass. in which various embodiments discussed herein are or can be implemented.

Absolute Insight is a big data analysis software program (e.g., web-browser based) that allows users to create and organize meaningful results from large amounts of data. The software is powered by, for example, algorithms and prepared models to provide users “one click” analysis out of the box.

In some embodiments, Absolute Insight allows users to control and process data with a variety of functions and algorithms, and creates analysis and plot visualizations. Absolute Insight may have prepared models and templates ready to use and offers a complete variety of basic to professional data messaging, cleansing and transformation facilities. Its Risk score and Ranking engine is designed so that it takes about a couple of minutes to create professional risk scores with a few drag and drops.

In some embodiments, the data analysis software provides a number of benefits, for example:
Unobtrusive. For example, the software may be browser-based with zero desktop footprint.
Deep Intelligence that allows the user to understand why things are happening
Predictive Intelligence: predict what will happen next
Adaptive Learning: system learns and adjusts based on actual results
Complete Analytics Workflow: intuitive analytics processes
Powerful Insights: immediate productivity gains with drag and drop
Data Science in a Box: quickly understand the significance of the data
Perceptive Visualizations: articulate analysis with meaningful visualizations
Seamless Data Blending: quickly connect disparate data sources
Simplified Analytics: leverage prebuilt analytic models
Robust Security: be confident your data and analysis are secure

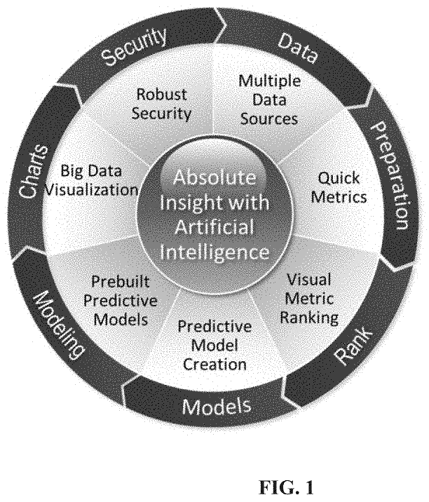

To that end, in some embodiments, Absolute Insight provides cloud enabled prebuilt data mining models, predictive analytics, and distributed in-memory computing. A summary of features provided by Absolute Insight is shown in FIG. 1.

The software allows users to begin by accessing data repositories. Users are then able to clean and generate aggregates and/or apply predictive templates and analyze results.

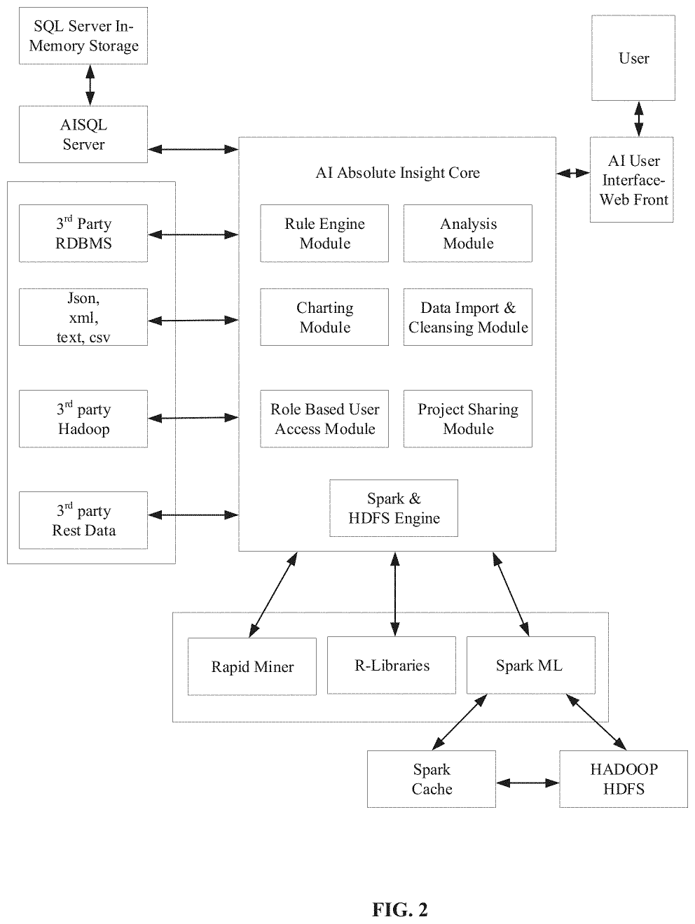

Absolute Insight's distributed architecture will be described further below and is schematically shown in FIG. 2. Among other things, Absolute Insight includes a core processing system (AI Absolute Insight Core) that includes various modules including a Rule Engine module, an Analysis Module, a Charting Module, a Data Import and Cleansing Module, a Role Based User Access Module, and a Project Sharing Module. These modules are discussed below. The core processing system also includes a Spark and HDFS Engine which represents an interface to a Spark distributed computing cluster supporting a version of the Hadoop Distributed File System (HDFS) upon which the core processing system runs in this exemplary embodiment.

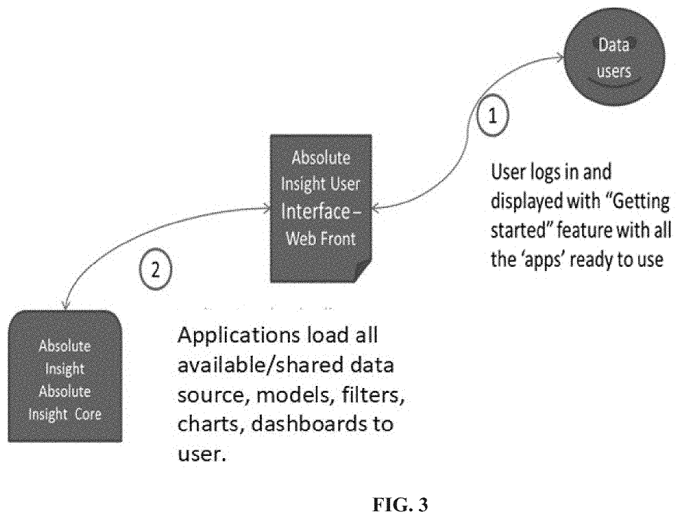

Various interactions between components in the distributed architecture are now described with reference to FIGS. 3-11. With reference to FIG. 3, at 1, a user interaction interface, e.g., a web-browser based interface, allows a user to log in. Once the user logs in, at 2, the “Get Started” feature (discussed in more detail below) is displayed and has ready-to-use ‘apps’. The interface is coupled to applicants that load all of the available and/or shared data sources, models, filters, charts, and dashboards to the user.

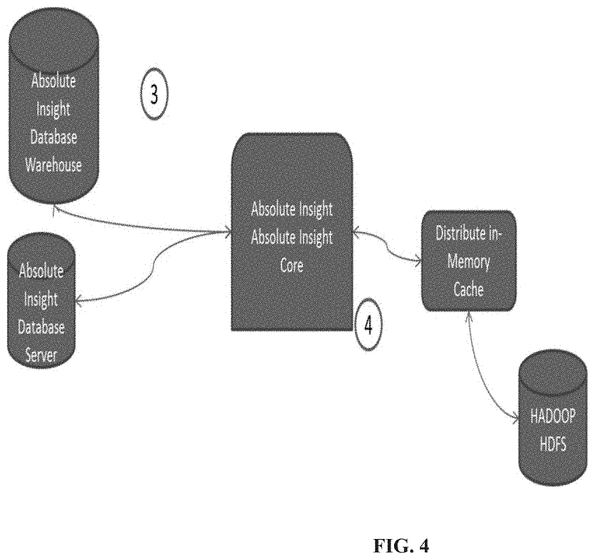

Then, with reference to FIG. 4, at 3, Absolute Insight obtains the list of all artifacts (saved and/or shared models, risk scores—rankings, charts and dashboards) from a Database. At 4, Absolute Insight also gets a list of cached processes, data and lists them for the user.

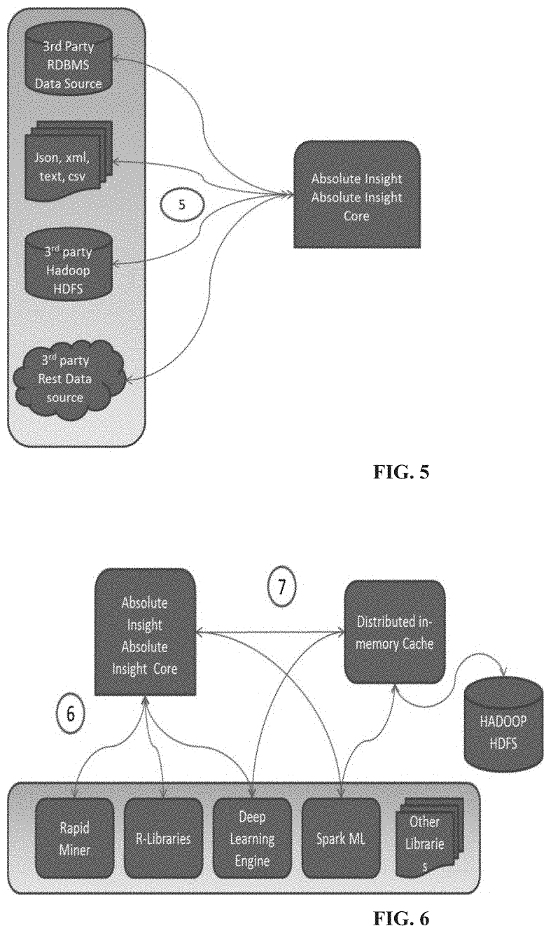

With reference to FIG. 5, at 5, Absolute Insight responds to a user request to read or bring in data from any source. Absolute Insight reads or copies the data to make it available for analysis.

With reference to FIG. 6, depending upon the app that the user activates, at 6, Absolute Insight may utilize the number of libraries, models, and templates involved in order to carry out the analysis. If the analysis involves machine learning, such as Deep learning, then the Deep Learning Engine is called at 7, which accesses the distributed in-memory cache and executes the learning process to build medical claim fraud predictive models. The best-suited medical claim predictive model is applied to unseen data to get fraud prediction results.

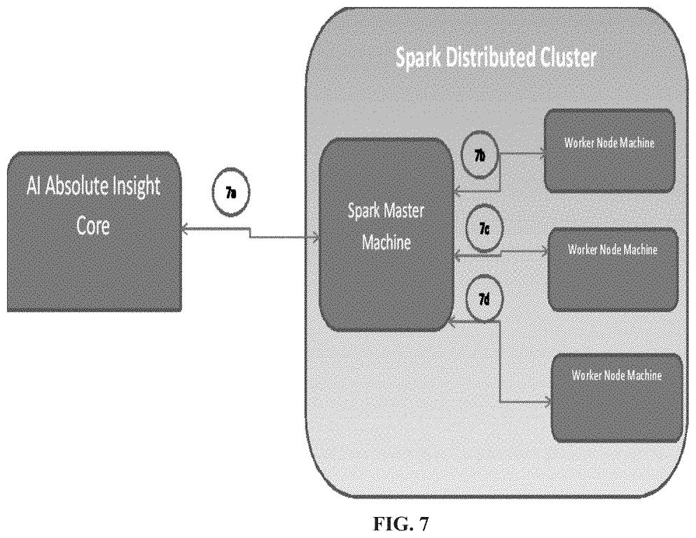

As depicted in FIG. 7, whenever a call is made to Spark for fraud detection (item 7a), the underlying call will be made to the cluster of machines (items 7b-7d) managed by the “Spark Master”

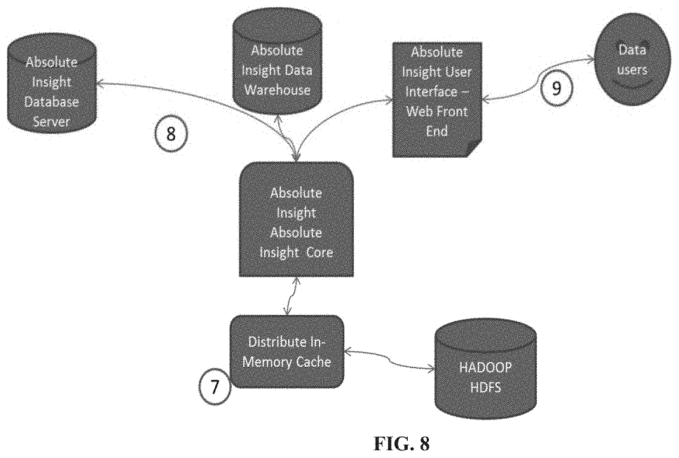

With reference to FIG. 8, once the results have been generated, they are pushed to the data warehouse and to the user interface at 8. At 9, the results are displayed to the user, who can further continue analysis of the results and/or share the results with peers, publish the results, and/or feed the results to the next process.

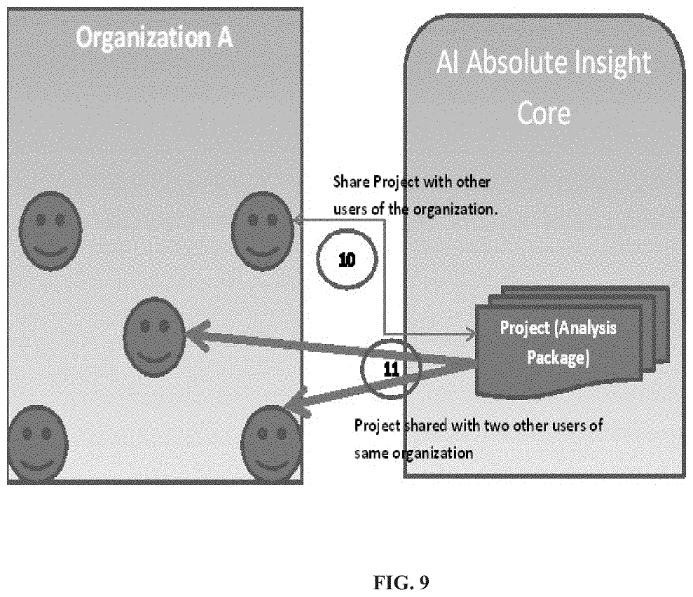

One major feature of Absolute Insight is its ability to share information within an organization and across organizations. Within an organization, the user can share a Project (Analysis Package) among other users as shown below in step 10 and 11, for example, as depicted in FIG. 9.

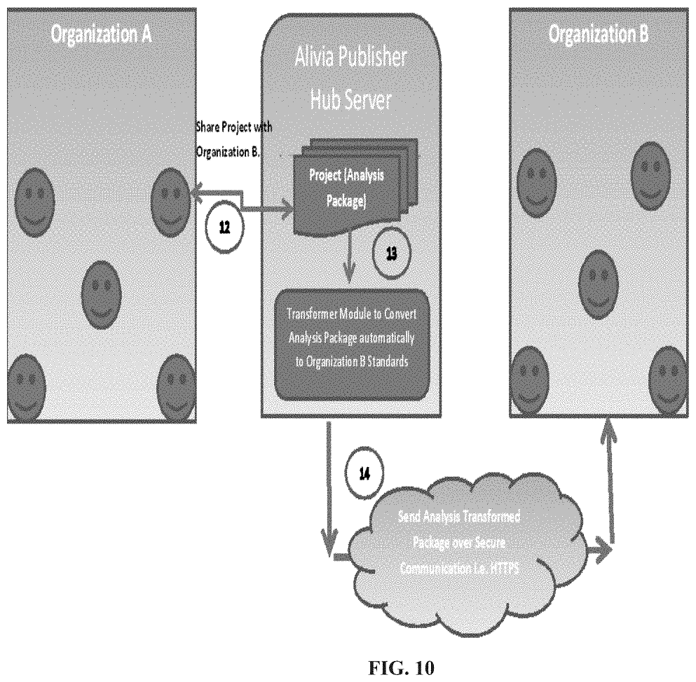

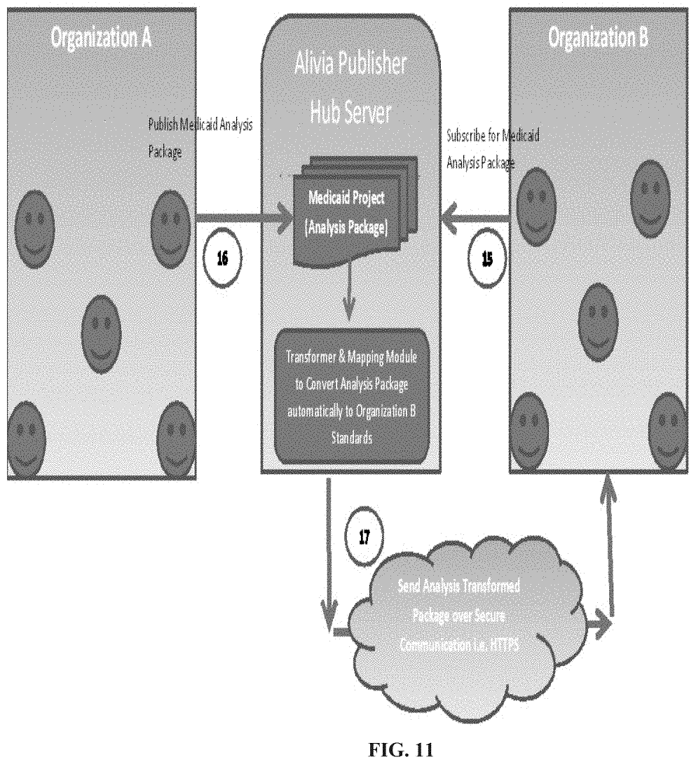

There are two modes for sharing projects among organizations:
1. A user from one organization can share a Project with other organization as shown below in steps 12, 13, 14, for example, as depicted in FIG. 10. In step 12, the user is sharing a project. In step 13, the Alivia Server Hub performs mapping and transformation for the organization, and then in step 14, sends the package to the other organization over a secure channel.
2. A publisher/subscriber mechanism can be used to share a project from one organization to other organizations, where organizations that have subscribed for a particular Analysis package will get it as soon as it is published by the other organization, for example, as depicted in FIG. 11.

### Interface

Absolute Insight's fraud detection interface is designed for ease of use. For example, the interface uses “drag & drop” and/or “plug & play” features. Each artifact, including data sources, filters, risk scores, models, templates, charts and/or dashboards, may be completely cohesive and pluggable to each other because of common input/output data ports. To that end, under the Modeling Tab the user may prepare their analysis model that can produce instant, reusable and/or schedulable results. All of the artifacts are listed and available in the modeling canvas to build reusable fraud processing template apps. Absolute Insight allows users to use one click ‘apps’. This provides a level of convenience to even novice users, who may drag and drop almost any kind of data source along any “fraud detection app” and it will do that analysis with no or minimal configuration.

### Deep Learning

In some exemplary embodiments, deep learning is used for healthcare fraud detection, such as for detecting medical claim fraud or other anomalies (e.g., doctors who over-prescribe certain drugs or treatments). In Absolute Insight, deep learning algorithms and models are available to use within templates. The deep learning templates are available to users as “plug-in” models. Deep learning models support “Big Data” analysis and, in certain exemplary embodiments, incorporate in-memory distributed computing and caching. Furthermore, Absolute Insight has learning templates that help to identify low level medical fraud patterns. Absolute Insight deep learning models help construct medical fraud indicators and more complex Medical Fraud processes.

Absolute Insight deep learning on distributed computing cluster makes computations highly scalable with extreme performance. The architecture can take hundreds of millions of medical claims, develop medical claim fraud features, process the feature creation using in-memory distributed processing, as each layer is processed the medical claim fraud features become more complex until it gets the picture of the entire medical fraud scheme and can classify the entity as fraudulent behavior.

Absolute Insight's in-memory cluster computing platform can take advantage of memory and CPUs, on all the network nodes available to the platform.

Absolute Insight Deep Learning uses multiple hidden layers and numerous neurons per layer, which are provided the medical claims feature set and the algorithm which identifies simple fraud indicators to complex fraud indicators.

In Absolute Insight, deep learning can process hundreds of epochs on the data (where one epoch represents a complete pass through a given data set) to minimize the error and maximize medical claim fraud classification.

Absolute Insight Deep learning may:
perform dimension reduction, classifier, regression and clustering attempting to mimic human brain modeled by neurons and synapses defined by weights.
identify simple medical fraud concepts and combine them to identify a whole medical fraud concept from simple medical indicators.
model high level medical fraud abstraction using a cascade of transformations.
identify simple medical fraud features to construct more complex representations of medical claim fraud in hidden layers and put together a whole picture representation identifying an entity as fraudulent or not.

Also, the process may reveal new methods of medical claim fraud analysis and refined automation of the identification of medial claim fraud.

Deep learning features finding patterns in extremely complicated and difficult problems. It has the potential to make huge contributions in detecting fraud, abuse, and waste in healthcare fraud detection.

In the context of healthcare fraud detection, the data usually includes the following parts. Claim data generally information such as the starting and the ending dates of the service, the claim date, the claim amount claim, and so on. Patient data has demographic information. Patients' eligibility data has information about the programs for which each patient is eligible or registered. Provider data has contacts of the providers, and providers' license and credential shows their qualifications. Contract data includes detailed rules in the insurance contracts.

In exemplary embodiments, two deep learning algorithms are applied, namely convolutional deep neural networks (CNN) and recurrent neural networks (RNN). These algorithms are combined to provide robust results.

The general steps of performing deep learning are now described with reference to FIGS. 78.

At 1, before running supervised deep learning, the application pre-processes the data, include labeling and computing metrics. For example, in the context of healthcare fraud detection, the application typically performs the following data pre-processing operations: (a) identify providers that have been excluded from Medicaid, Medicare, or insurance companies for fraud, waste, or abuse behavior; (b) perform breakout detection in the time-series records of each provider to identify statistically significant anomalies, e.g., based on the amount claimed per month, and label the breakouts periods as the times when the provider likely conducted fraud, waste, or abuse in healthcare; (c) compute metrics based on domain knowledge (deep learning models can determine useful metrics through analysis of the data, but it is still beneficial if the application can provide a base set of known metrics, as pre-computing metrics can save computation and iteration time in deep learning and can make the results more easily interpretable); and (d) connect the resulting data source to an Analyzer Operator (discussed below).

At 2, deep learning is called by the Analyzer Operator, which sends a request to identify algorithms to use for a given dataset (described below with reference to FIG. 77, Analyzer Operator diagram, step 6).

At 3, the deep learning algorithms are executed, the performance metrics are used, and parameters are tuned (described below with reference to FIG. 77, Analyzer Operator diagram, steps 7 to 10). Specifically, for deep learning, many parameters are tuned for optimal predictive models. For example, the number of hidden layers, the number of neurons in the layers, the epochs, the learning rate, the activation function, and others are optimized.

At 4, the application creates an ensemble of models. Each component in the ensemble is a deep learning model focusing on a specific type of fraud (e.g., frauds with a group of procedure codes, pharmacy drug codes, etc.). These models are trained in sequence, and results from earlier models are fed as inputs to later models. This allows more accurate modeling of fraud occurrence patterns and complex fraudulent relationships, and thus provides higher-quality predictions.

In some embodiments, the software may use distributed computing in core memory and utilize in-memory Map & Reduce to perform the analysis and quickly identify medical claim fraud. Clusters of computers may be used to process and calculate, but some embodiments also use in-memory caching over the cluster of nodes to do in-memory data processing. Machine learning algorithms may be computed on distributed computing platform, thus enabling the creation of the medical fraud detection “apps” for users of the Absolute Insight software.

It should be understood that in some embodiments, all the templates and models are reusable, embeddable, and schedulable on events or on time. Fraud detection templates and models, for “one click” analysis, are available as “apps” in the “Get Started” tab. This will be discussed later in the document. All key important aspects of the application have been detailed below.

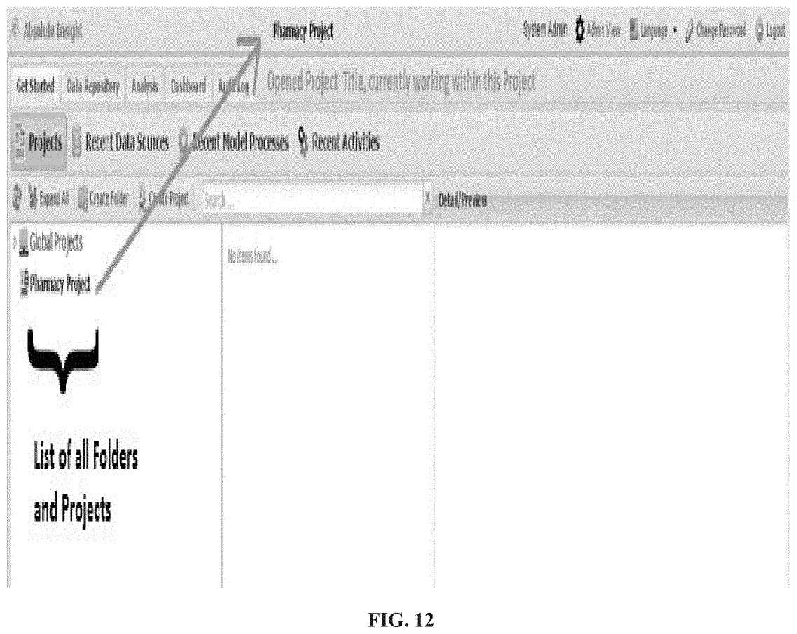

FIG. 12 is a sample annotated screen that shows various operations that can be performed by a user. Various exemplary embodiments are described herein with reference to “tabs” shown in the sample screen of FIG. 12. For example, from the sample screen shown in FIG. 12, a user can select from tabs labeled “Get Started,” “Data Repository,” “Analysis,” “Dashboard,” and “Audit Log.” Each of these tabs is discussed below.

### Get Started

With reference again to FIG. 12, when the user selects the “Get Started” tab, the user is presented with various options including “Projects,” “Recent Data Sources,” “Recent Model Processes,” and “Recent Activities.” In one exemplary embodiment, the “Recent Data Sources” tab shows a grouping of recently created data sources, a grouping of recently used data sources, and a grouping of recently shared/published data sources. Similarly, the “Recent Model Processes” tab shows a grouping of recently created processes, a grouping of recently used processes, and a grouping of recently shared/published processes. Similarly, the “Recent Activities” tab shows a grouping of recently created activities, a grouping of recently used activities, and a grouping of recently shared/published activities. The “Projects” tab is discussed below.

### Projects

Projects provide the Predictive Processes and Analysis packages that can be shared across organizations, divisions, departments, or groups. They can also be shared publically. In exemplary embodiments, this sharing is strictly governed by security implementations so that data may remain private among sharing entities.

The Analysis package is mostly mapped by wizard, when shared among different business domains then target mappings will help to transform one domain data to another seamlessly.

Project is like a workspace where all the work inside the Absolute Insight application is saved. Whatever the user creates in other parts of the application will be saved in one of the projects, which typically will be the currently opened project. FIG. 12 shows a sample screen with the “Projects” tab selected. This sample screen lists all folders and projects. In this example, there is one folder entitled “Global Projects” and one Project entitled “Pharmacy Project.” In this example, the title on the top bar indicates that the “Pharmacy Project” is the currently opened project and all activities done within the application will be saved under that project.

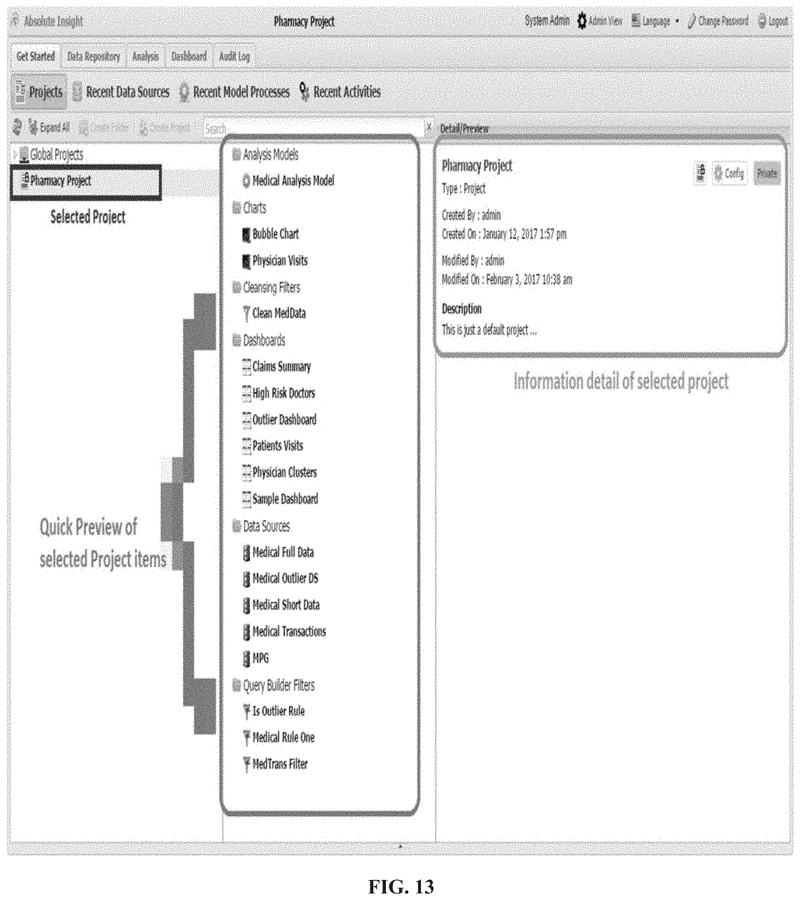

If the user wants to view the items currently residing in a particular project or wants to view information regarding a particular project, then the user just needs to click on that particular project, for example, as depicted in the sample annotated screen shown in FIG. 13. In this example, upon the user clicking on “Pharmacy Project,” a quick preview of selected Project items is displayed along with an information detail of the selected project.

### Creating a New Project or Folder

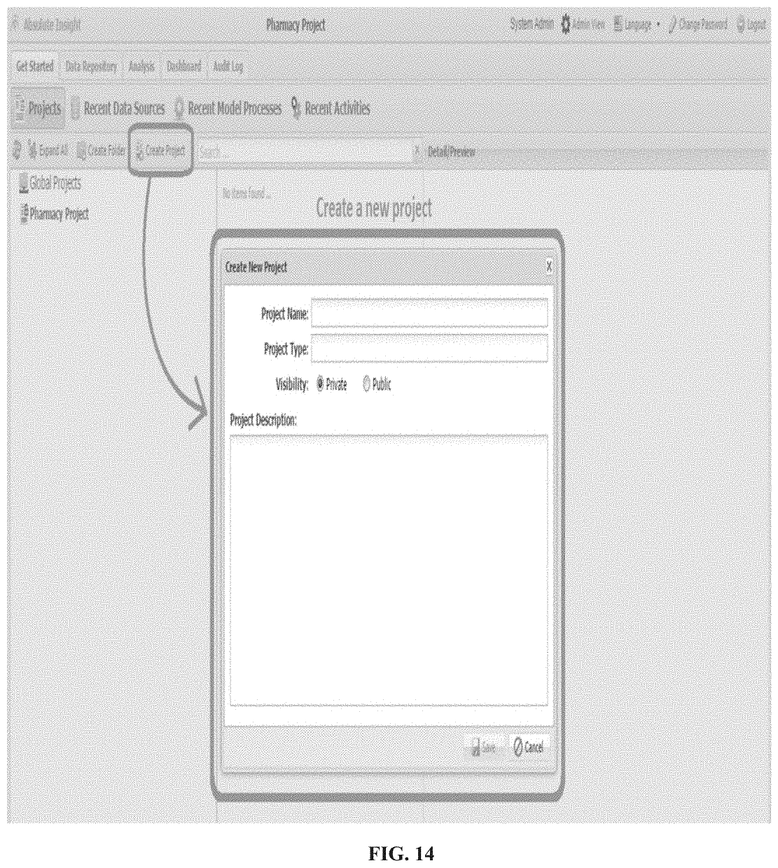

From certain screens, such as the sample screen shown in FIG. 12, the user can create a new project by selecting “Create Project,” for example, as depicted in the sample annotated screen shown in FIG. 14. Among other things, this brings up a dialog box in which the user can enter information such as the project name, the project type, the project visibility (e.g., private or public), and a project description. Similarly, from certain screens, such as the sample screen shown in FIG. 12, the user can create a new folder by selecting “Create Folder.”

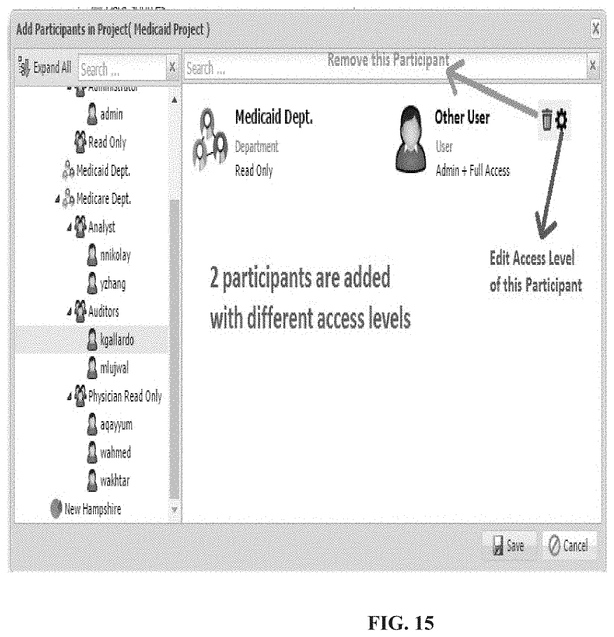

FIG. 15 shows a sample annotated screen with two participants added to a project called “Medicaid Project,” namely a “Medicaid Dept.” participant in which group members are given read-only access and an “Other User” participant given administrator and full access. Participants can be added to a project via a popup window. The user can select a particular participant and perform certain functions, such as removing the participant from the project (e.g., by selecting the trash can icon) or editing the access level of the participant (e.g., by selecting the gear icon).

### Absolute Insight (AI) Modules

AI is built in layers and services, and is divided into various vertical “tiers” which are made up of multiple modules, which combine to give a seamless service to the user.

### Base Modules

Exemplary embodiments typically include various types of base modules to process data and display results in various formats. The base modules may include such things as:
Rules Engine
Security Module
Models & Operators Engine
Dashboard
Query Builder
Data Repository
Ranking
Charting Engine

### Data Repository

In this module, users are able to create new data sources by connecting to various types of files and databases. They can view all the data sources that have been previously created, and they can manage those data sources, e.g., update connection information, or rename, refresh or delete them. In addition, the application shows meta-data details about a data source in the Detail View, when one is selected from the list of data sources.

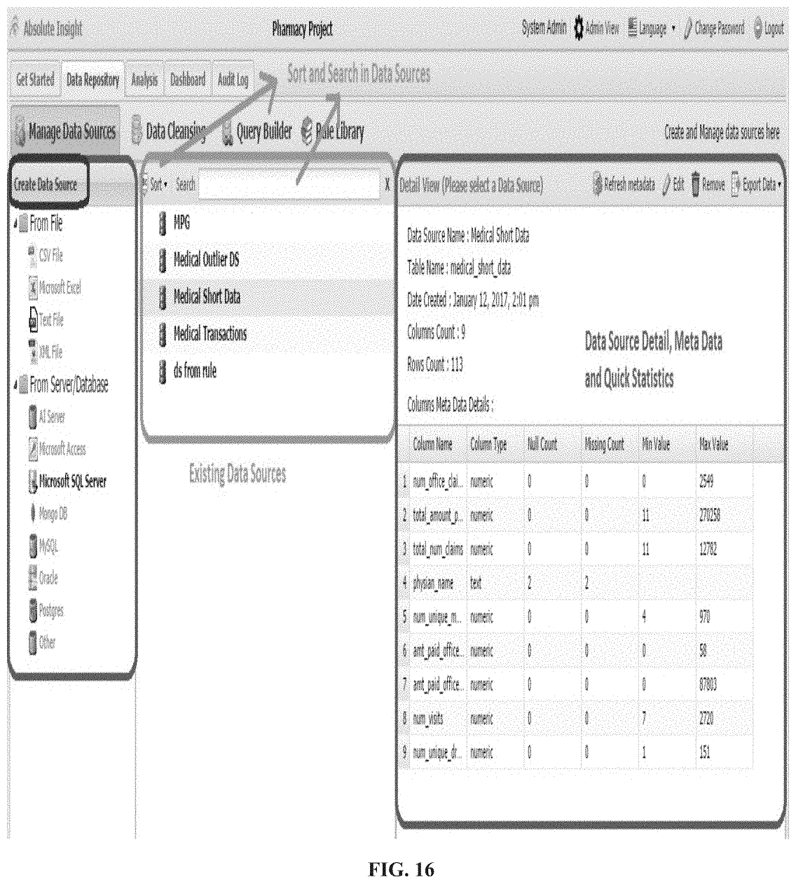

FIG. 16 shows a sample annotated Data Repository screen, such as might be presented when the user selects the “Data Repository” tab. When the user selects the “Data Repository” tab, the user is presented with various options including “Manage Data Sources,” “Data Cleansing,” “Query Builder,” and “Rule Library.” The sample screen shown in FIG. 16 shows sample information of the type that might be displayed when the user selects the “Manage Data Sources” tab. Here, the user is presented with a list of data sources and a “Create Data Source” button, a list of existing data sources with controls to sort and search the data sources, and a detail view presenting meta-data details about a selected data source (in this case, the data source entitled “Medical Short Data”).

### Snap Shot Grid

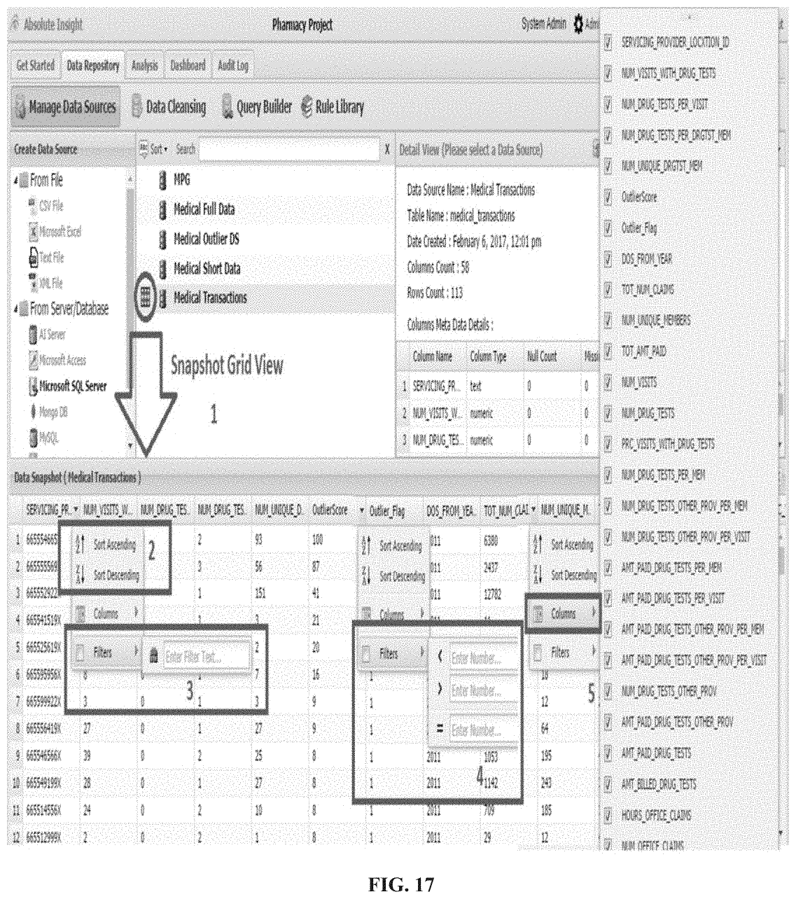

Users can also take a quick peak into the actual data in order to understand what is inside the data source. This can be done by clicking the “grid” icon available for each data source in the list, as depicted in the sample annotated screen shown in FIG. 17 (item 1). From this screen, the user can sort and filter data in each of a number of columns (items 2-5).

### Data Cleansing

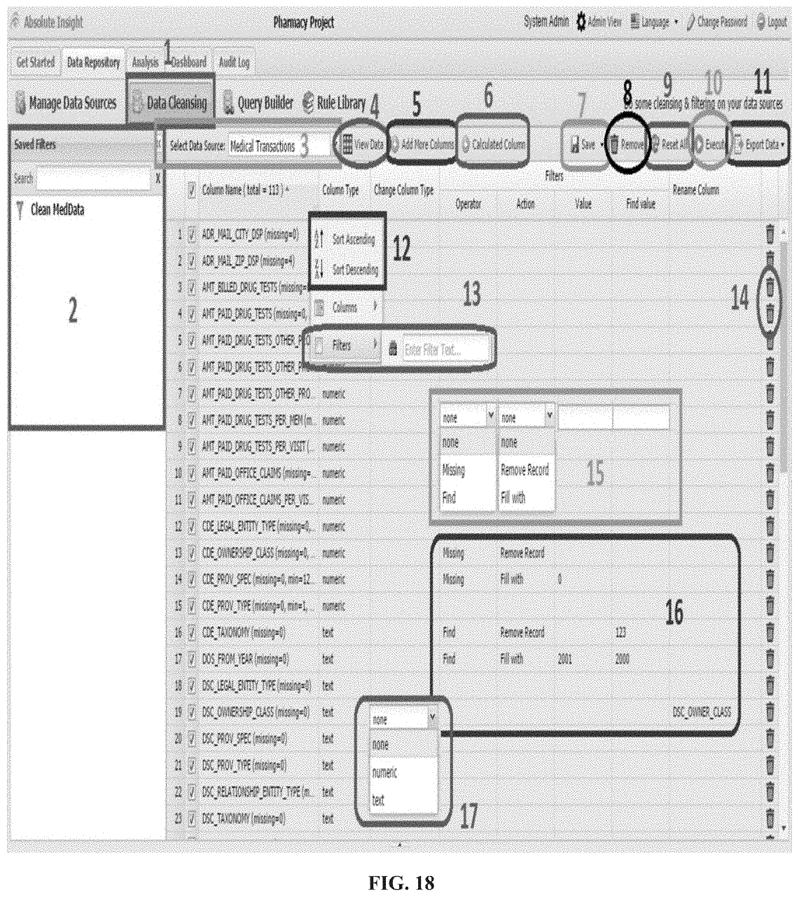

FIG. 18 shows a sample annotated screen such as might be displayed when the user selects the “Data Cleansing” tab (item 1). Here, users can quickly do the cleanup and apply filtering on the data in several ways within few clicks. Available data sources are accessible from the drop-down list.

The following is a brief description of the various items highlighted in FIG. 18:
Item 1 allows the user to Switch to Data Cleansing view.
Item 2 is a List of already created cleansing filters.
Item 3 allows the user to Select a data source in order to create a cleansing filter.
Item 4 allows the user to Quickly view a snap shot of the selected data source.
Item 5 allows the user to Add more columns, if they were deleted accidentally.
Item 6 allows the user to add calculated columns, e.g., apply some functions or merge of multiple columns into a single one.
Item 7 allows the user to Save current filter configurations as cleansing filter.
Item 8 allows the user to Remove the selected cleansing filter.
Item 9 allows the user to Reset all configuration done in data cleansing window.
Item 10 allows the user to Execute cleansing configuration of selected data source and view the results in snapshot grid view.
Item 11 allows the user to Export current cleansing configuration in various formats, such as CSV, and save as application data source.
Item 12 allows the user to Sort data source columns by name in ascending or descending order.
Item 13 allows the user to Filter data source columns by name.
Item 14 allows the user to Remove column so that it will not be included in the execution results.
Item 15 provides Filtering options available in the Data Cleansing view.
Item 16 shows some Examples of filters used.
Item 17 allows the user to Change column type from text to numeric or numeric to text.

### See Copy/Paste Cleansing Function Usage

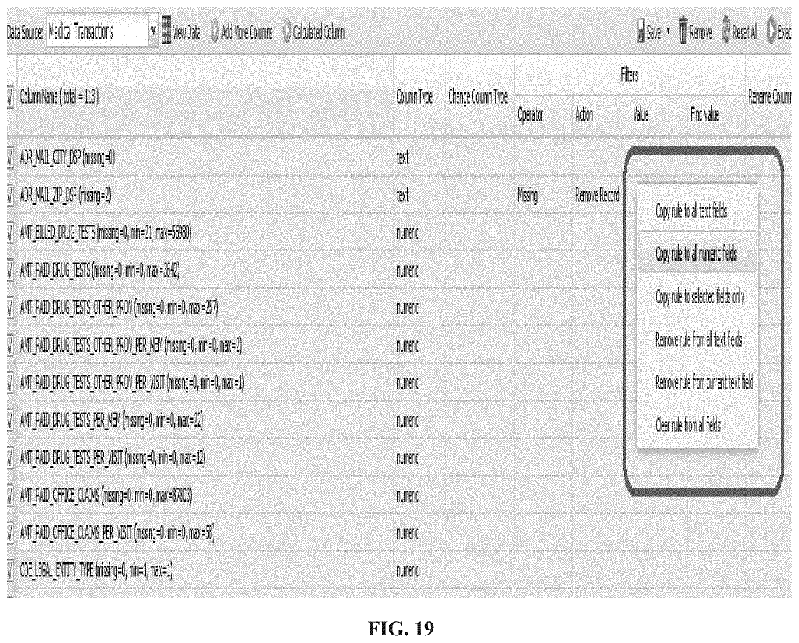

FIG. 19 is a sample annotated screen with a popup window allowing the user to choose if and where a particular rule is applied.

### Query Builder

FIG. 20 is a sample annotated screen such as might be displayed when the user selects the “Query Builder” tab. Among other things, this sample screen displays the saved Query Builder filters and rules. The Query Builder provides a very easy, yet comprehensive way to perform data mining and develop complex aggregations to squeeze desired information out of a large data-set. It also shows the Data Scientist a first-hand view of how the query looks in the “Query Editor” and it gives an option to edit it directly as well.

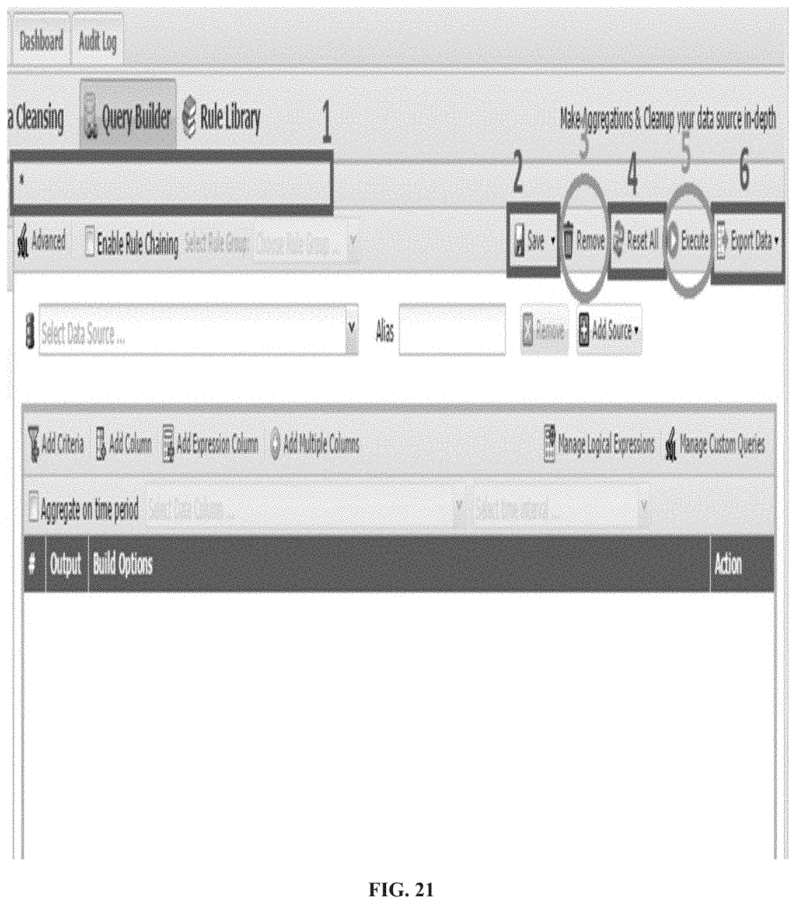

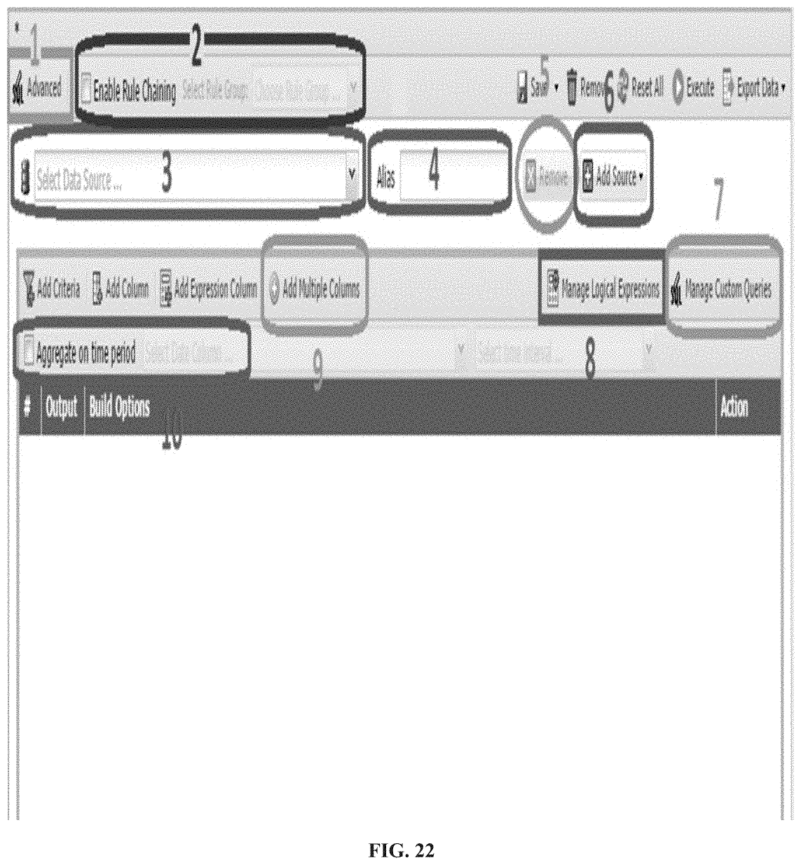

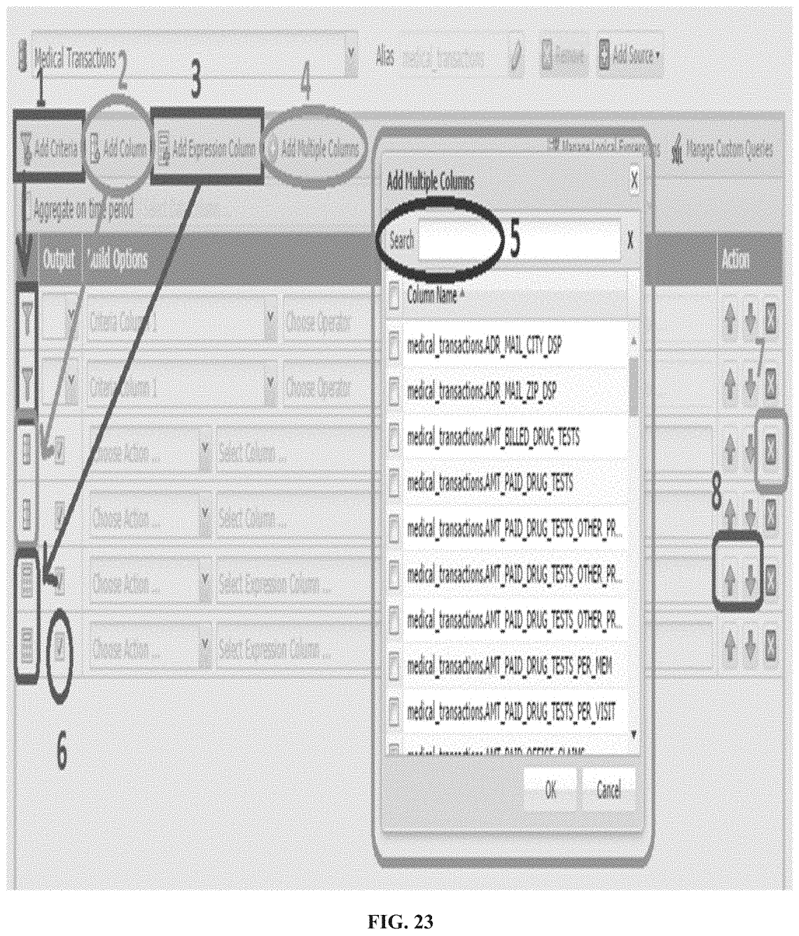

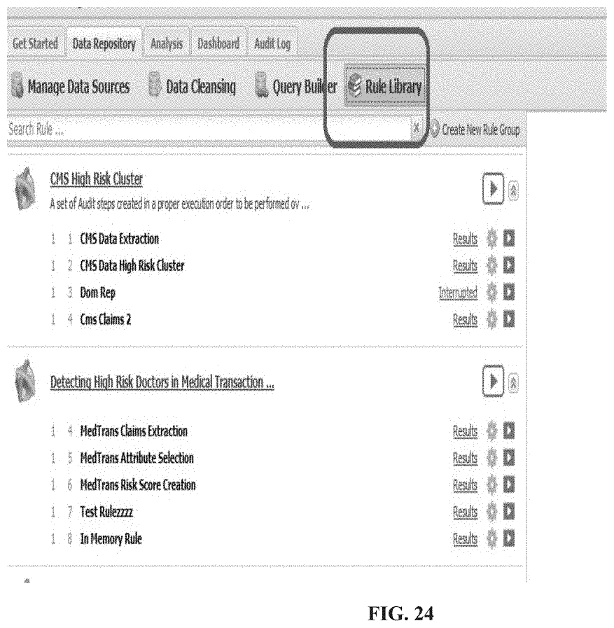

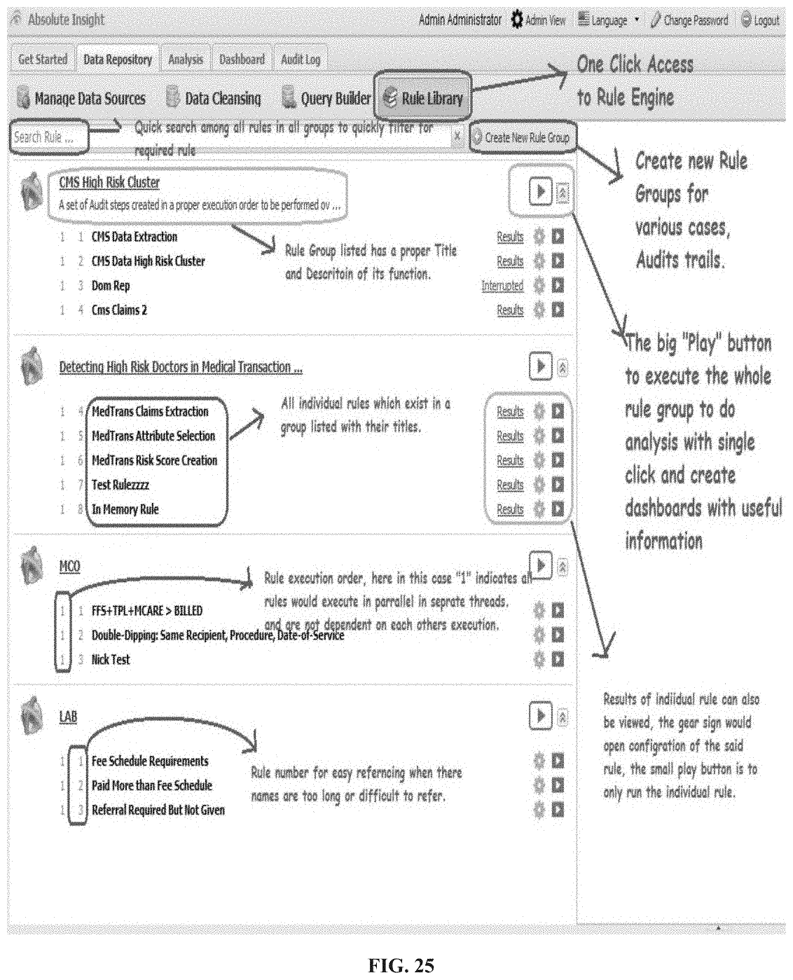

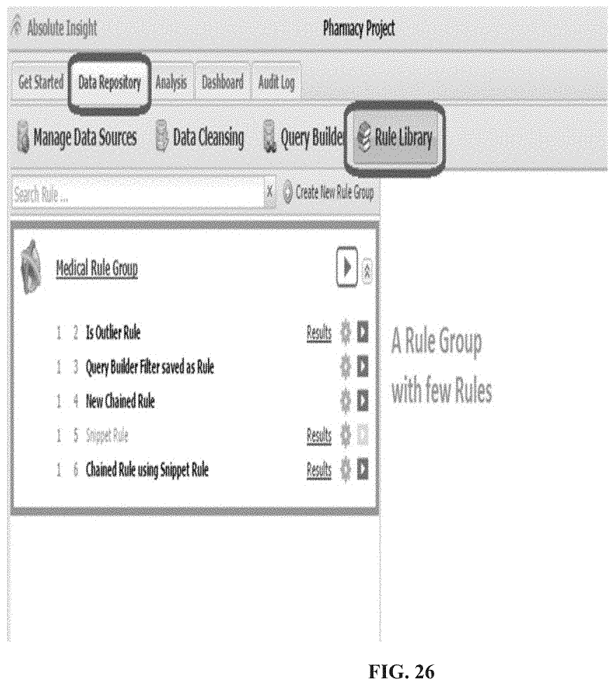

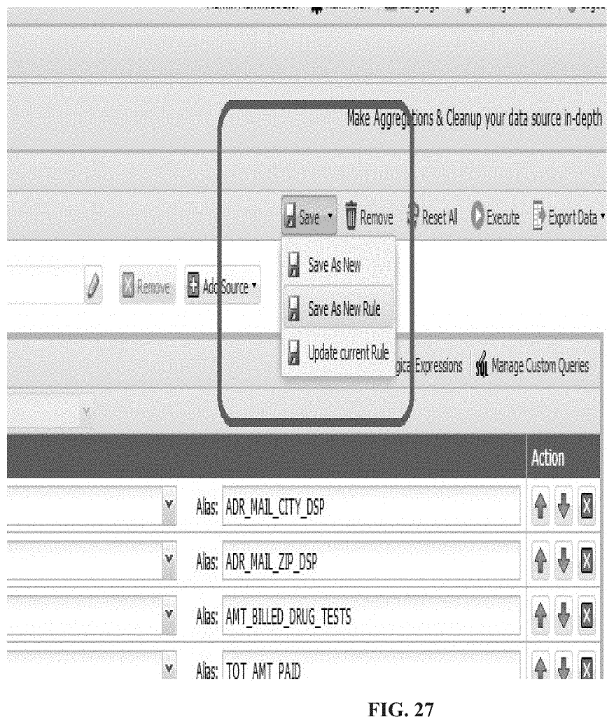

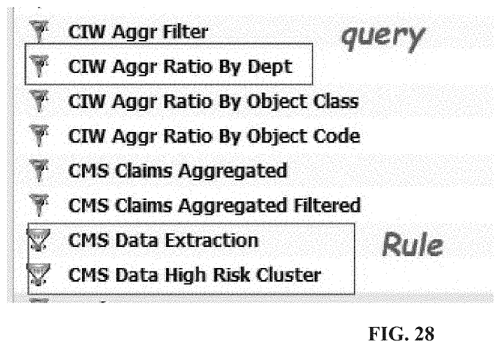

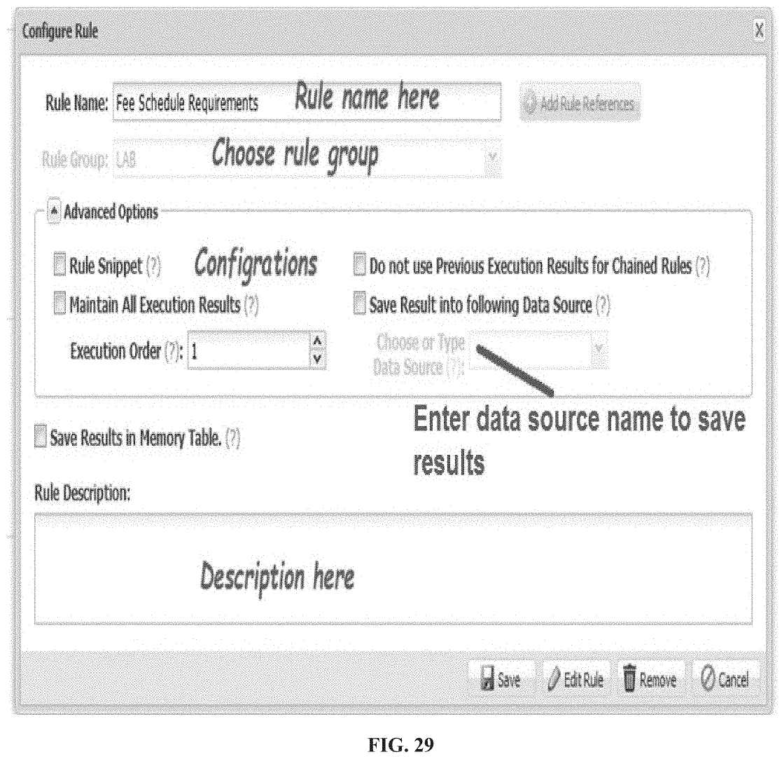

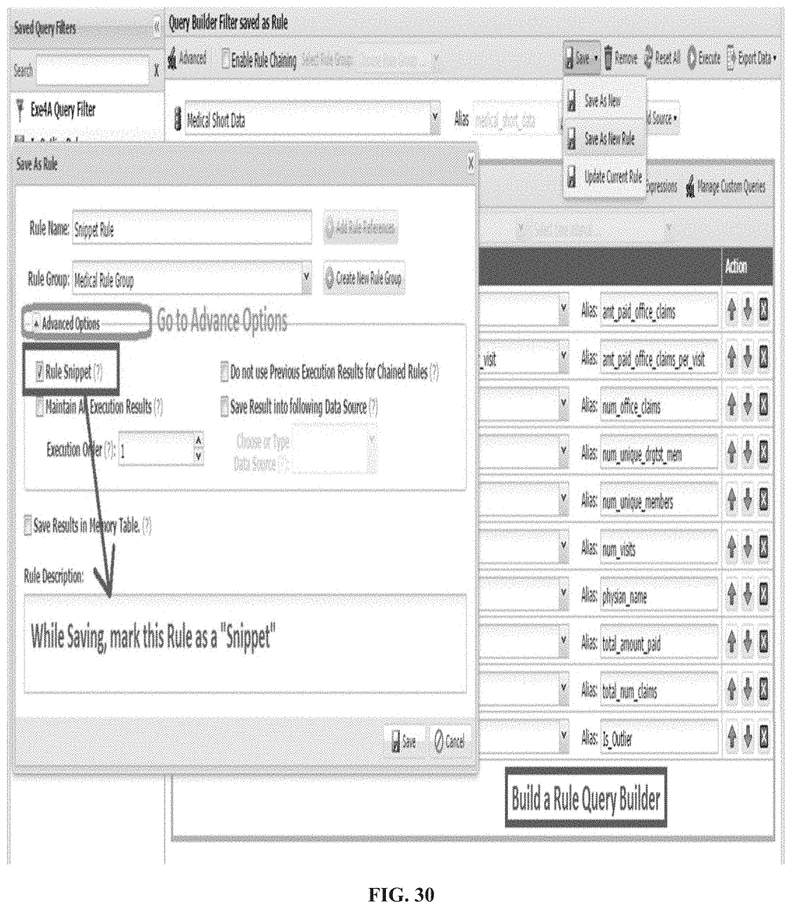

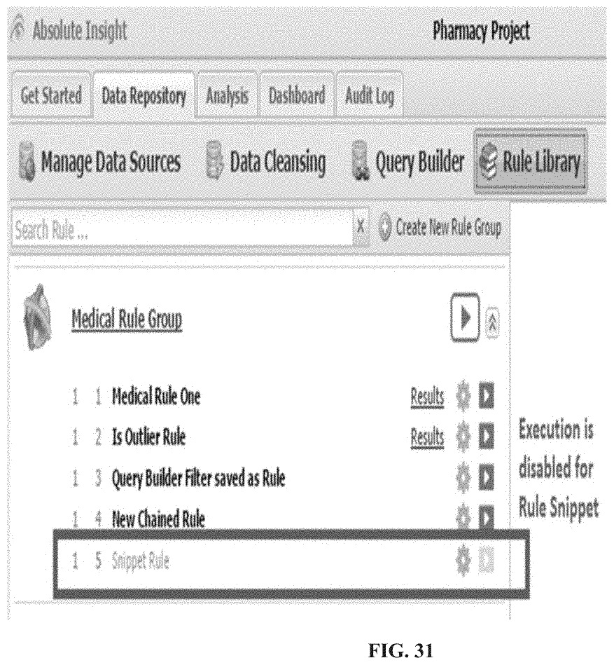

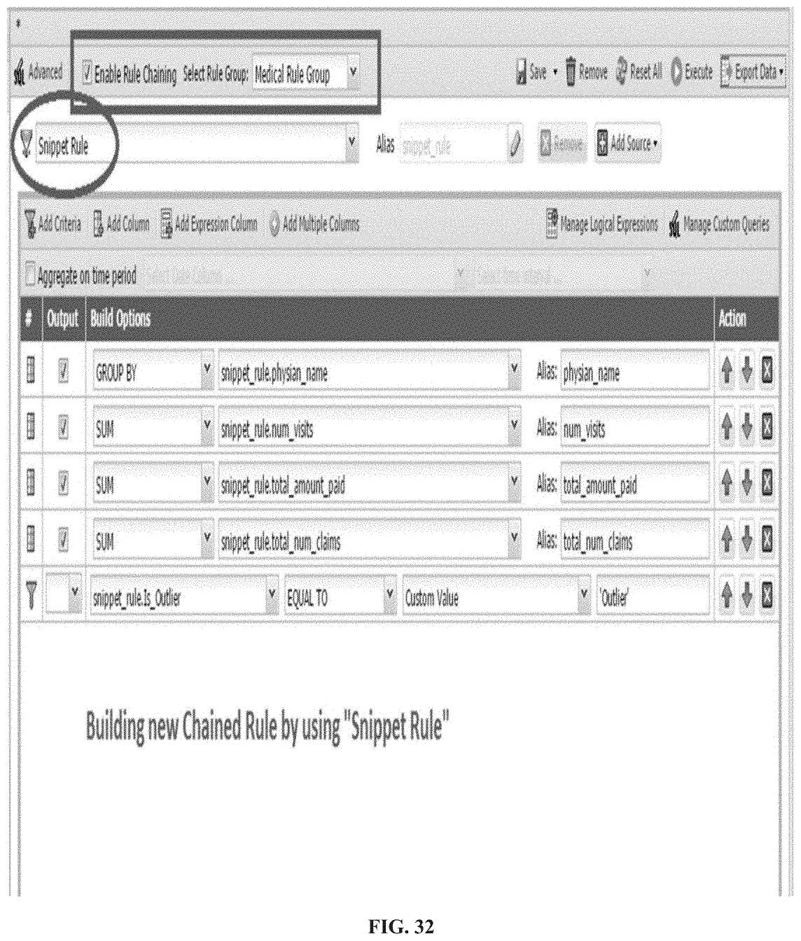

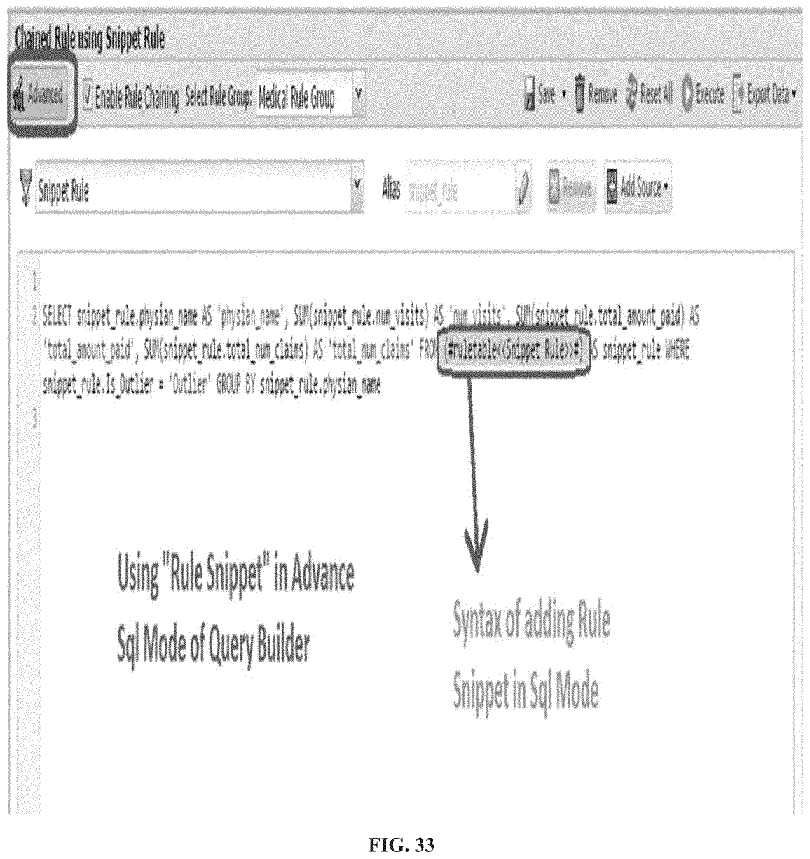

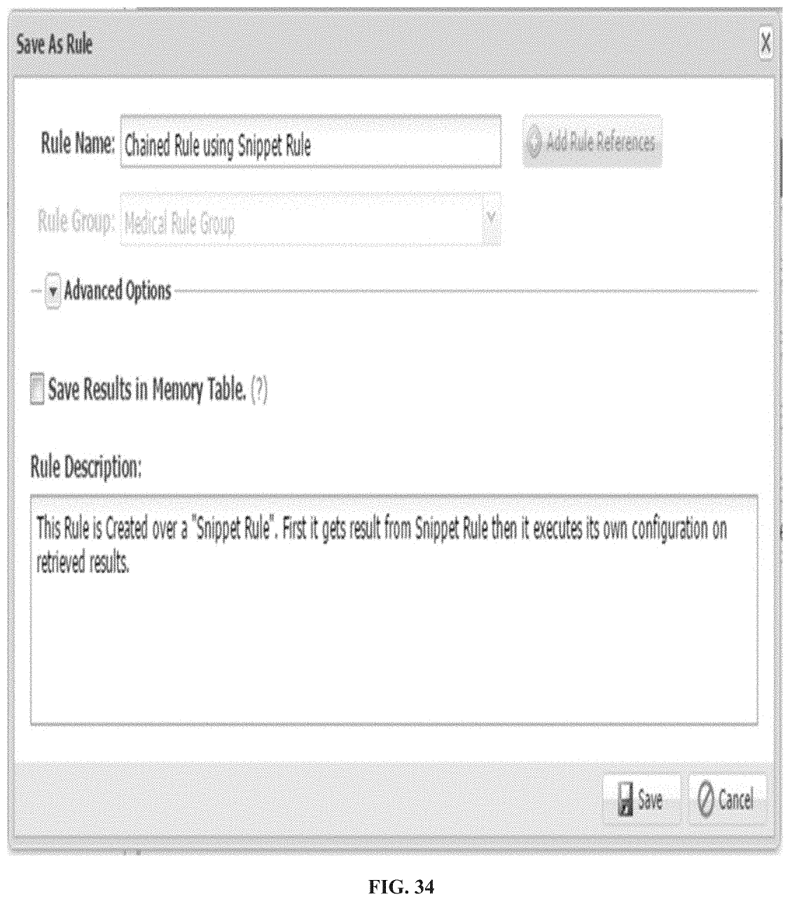

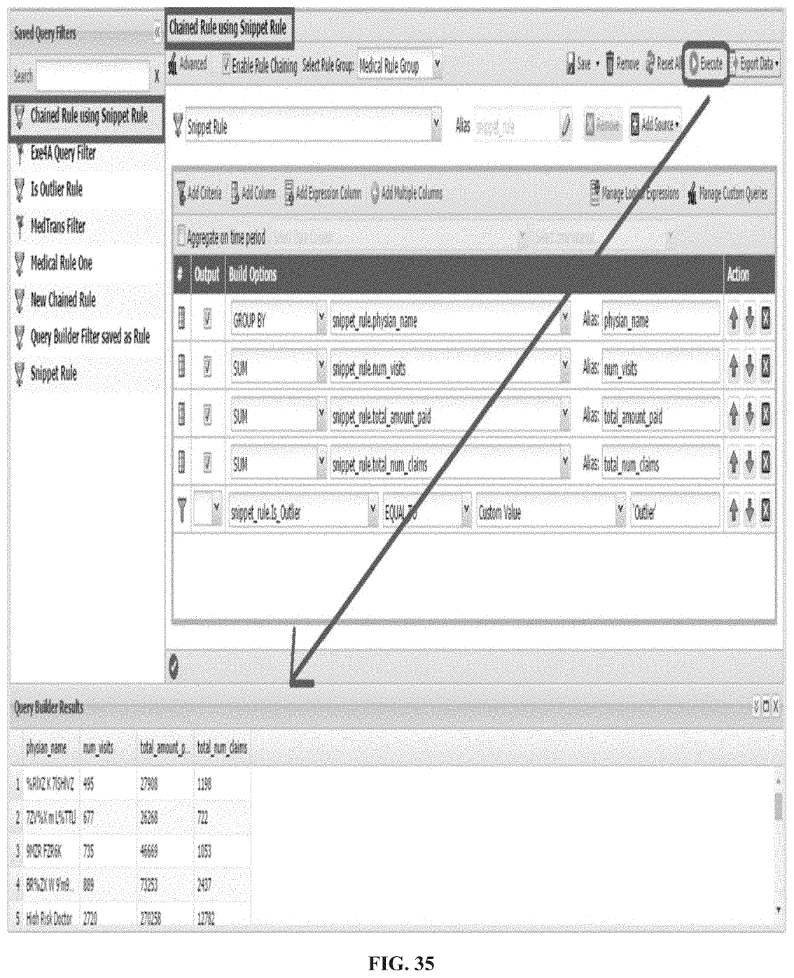

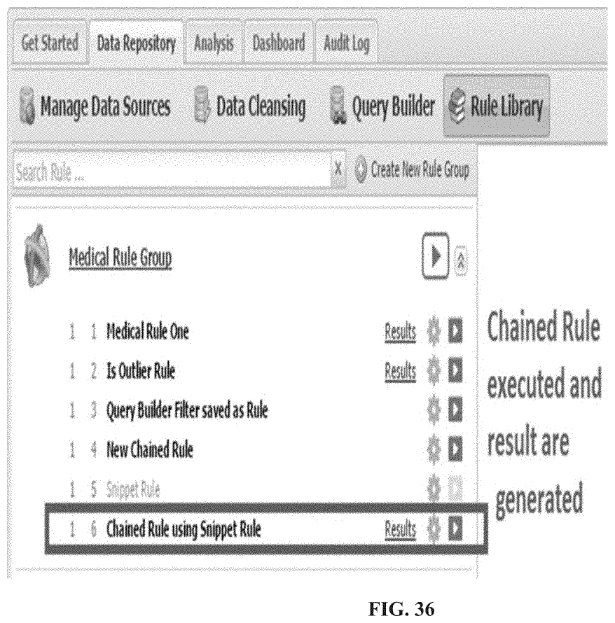

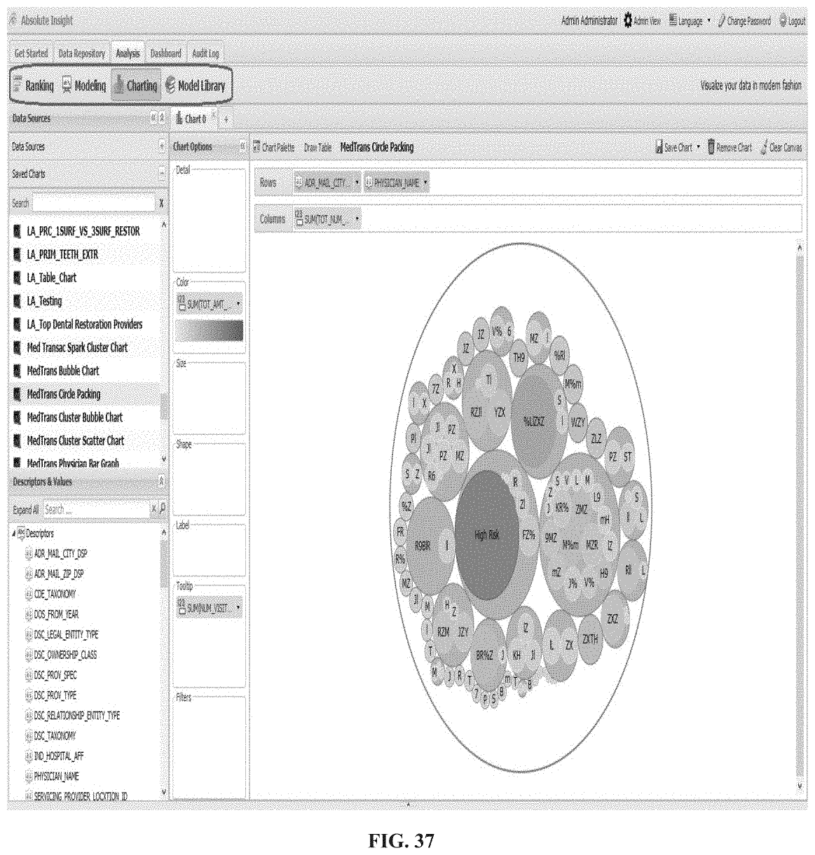

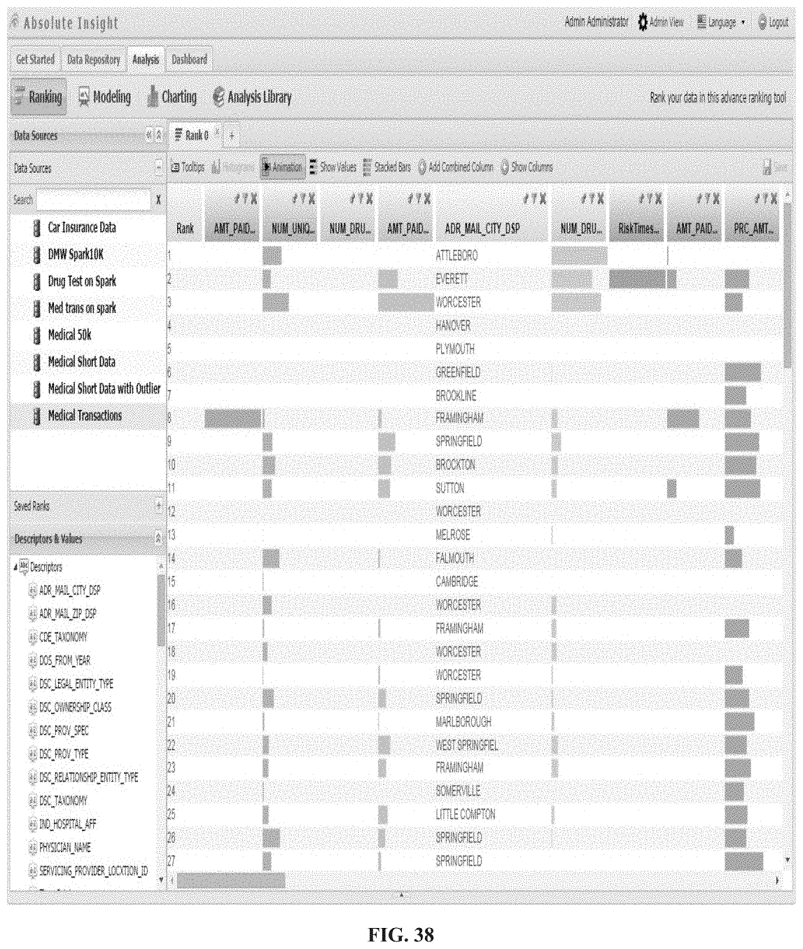

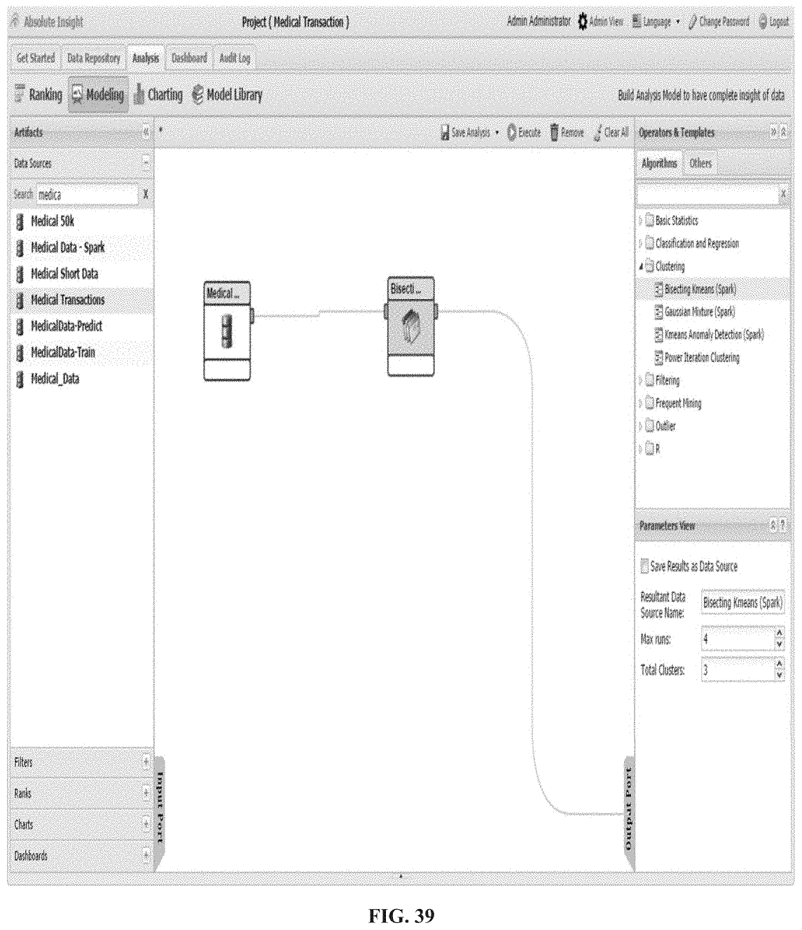

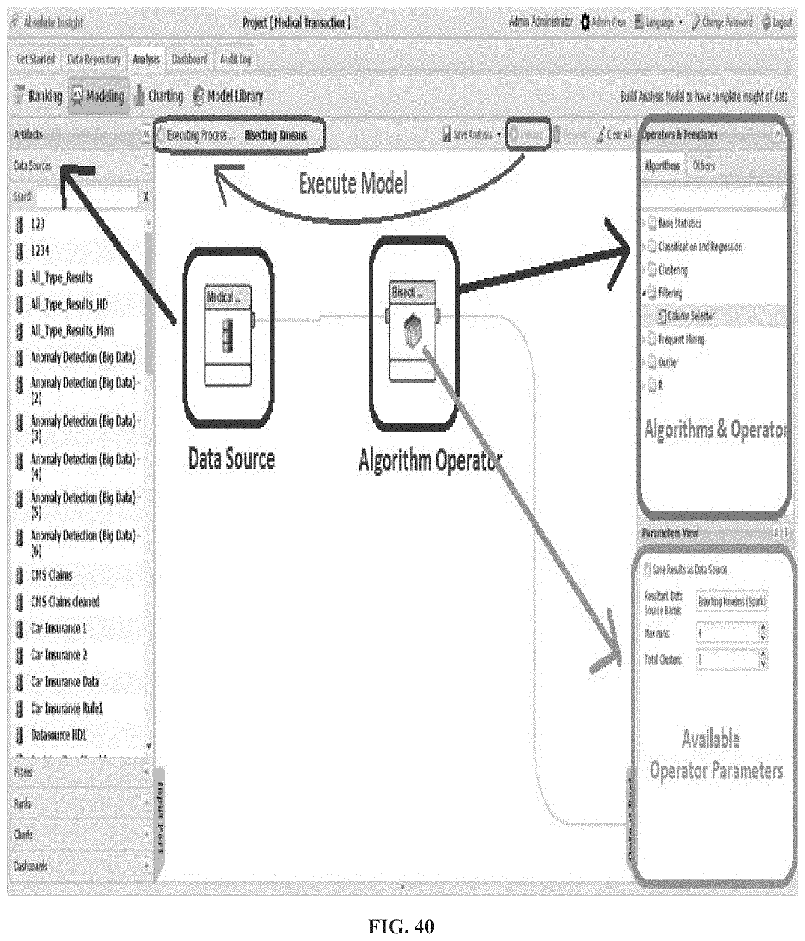

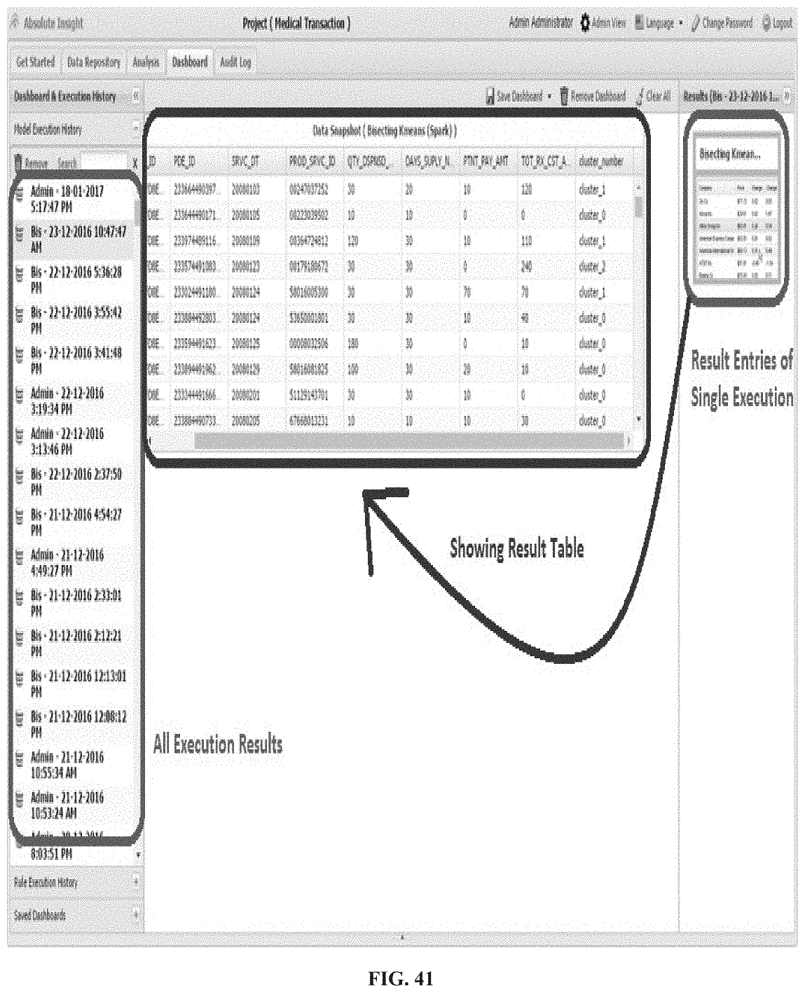

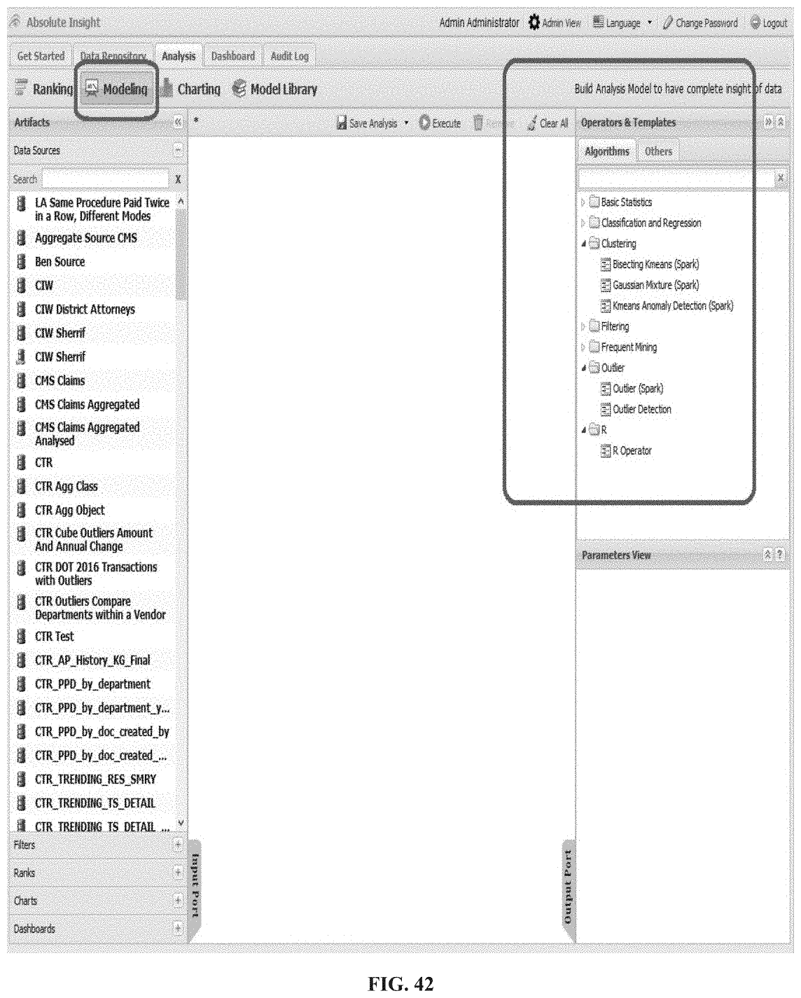

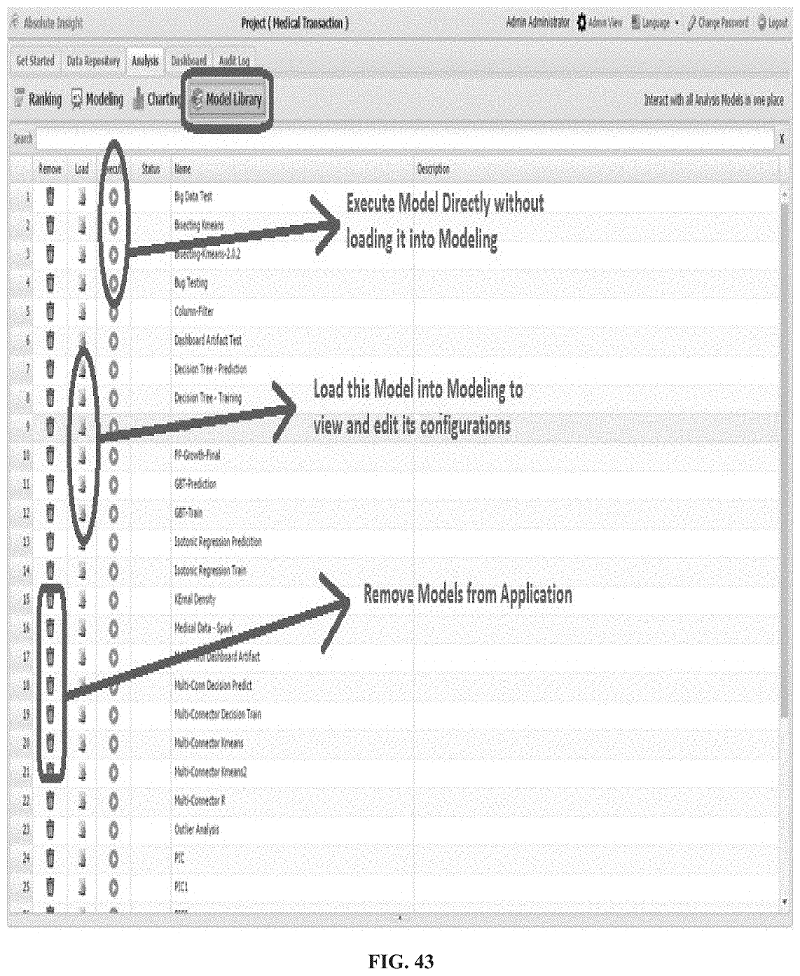

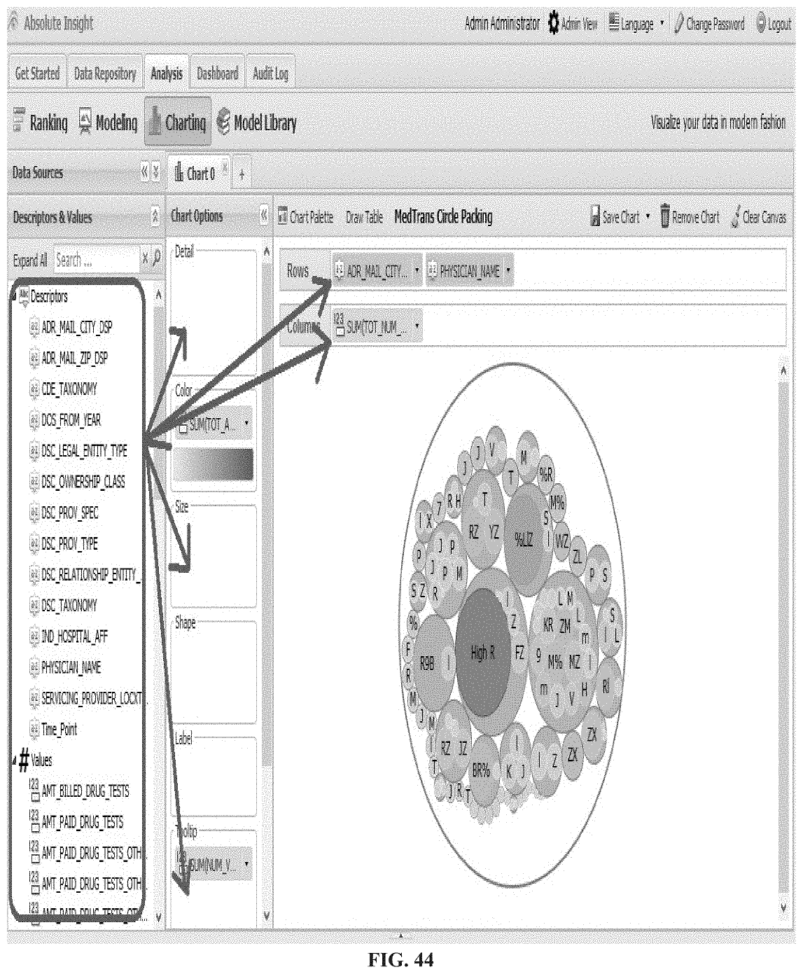

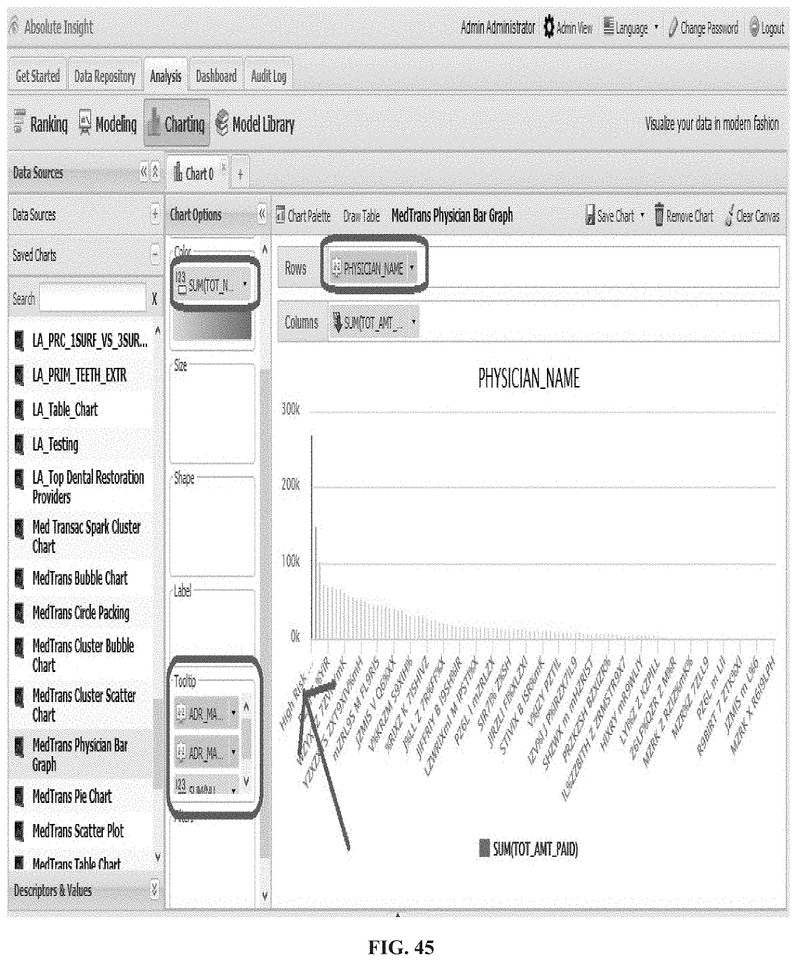

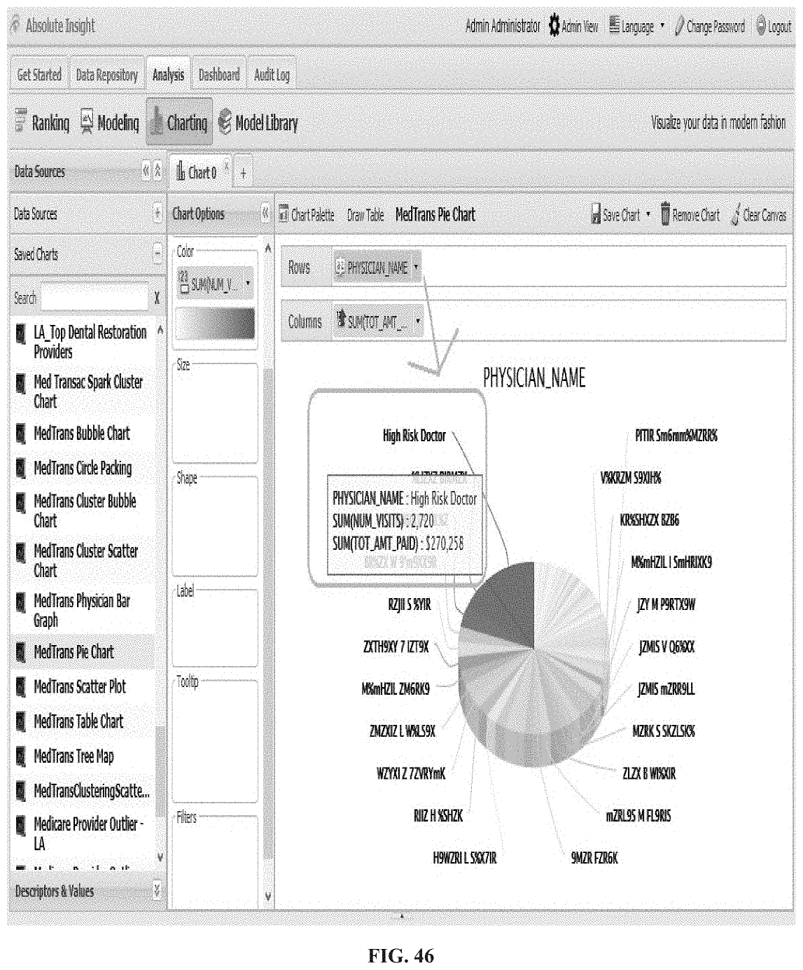

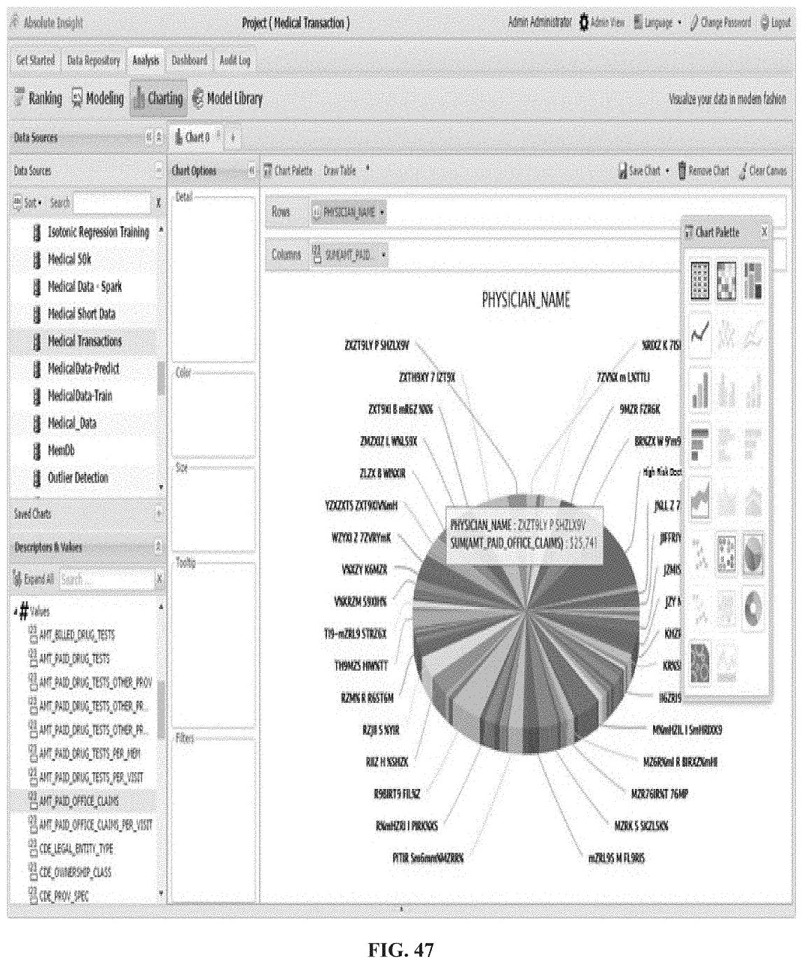

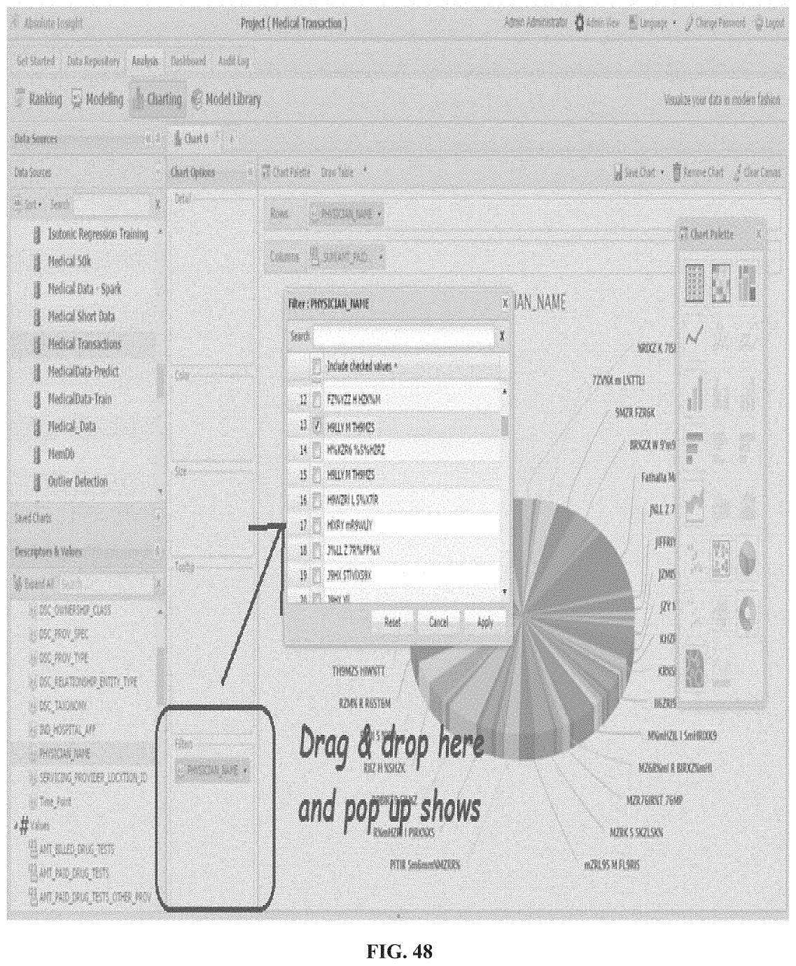

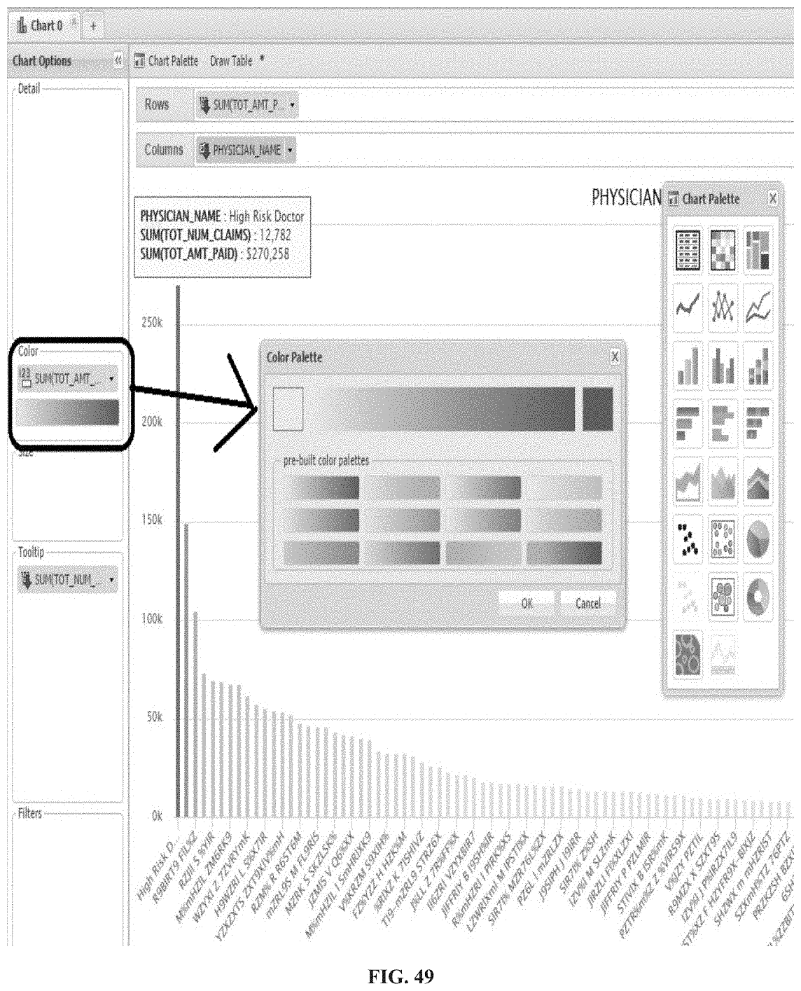

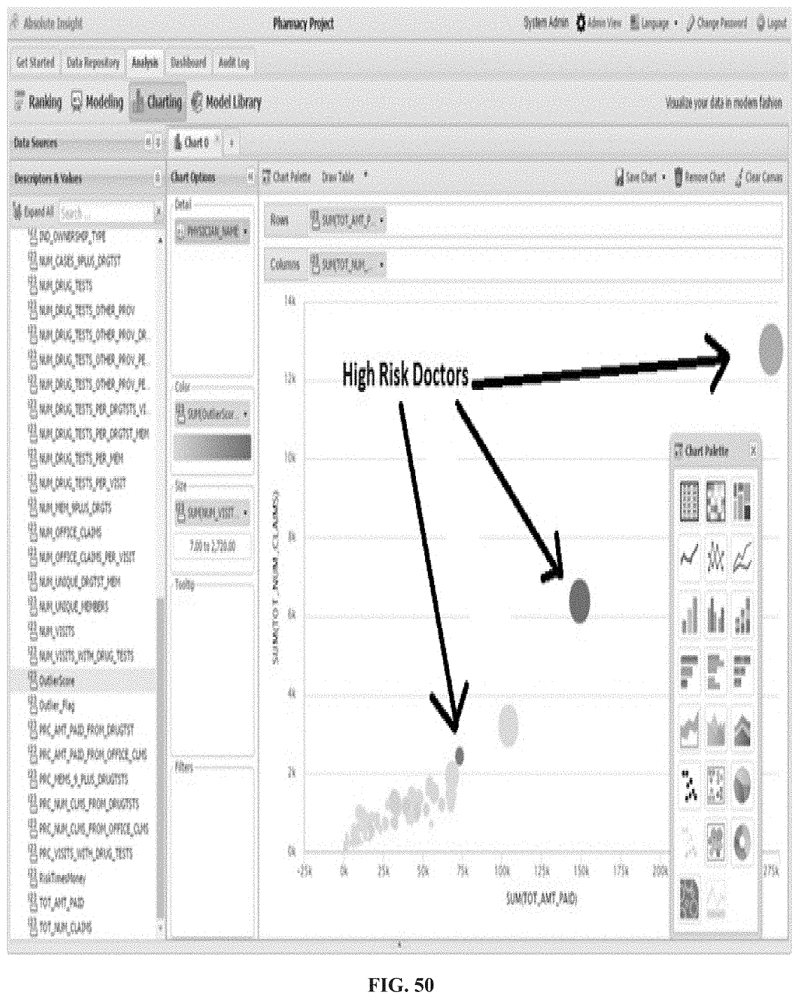

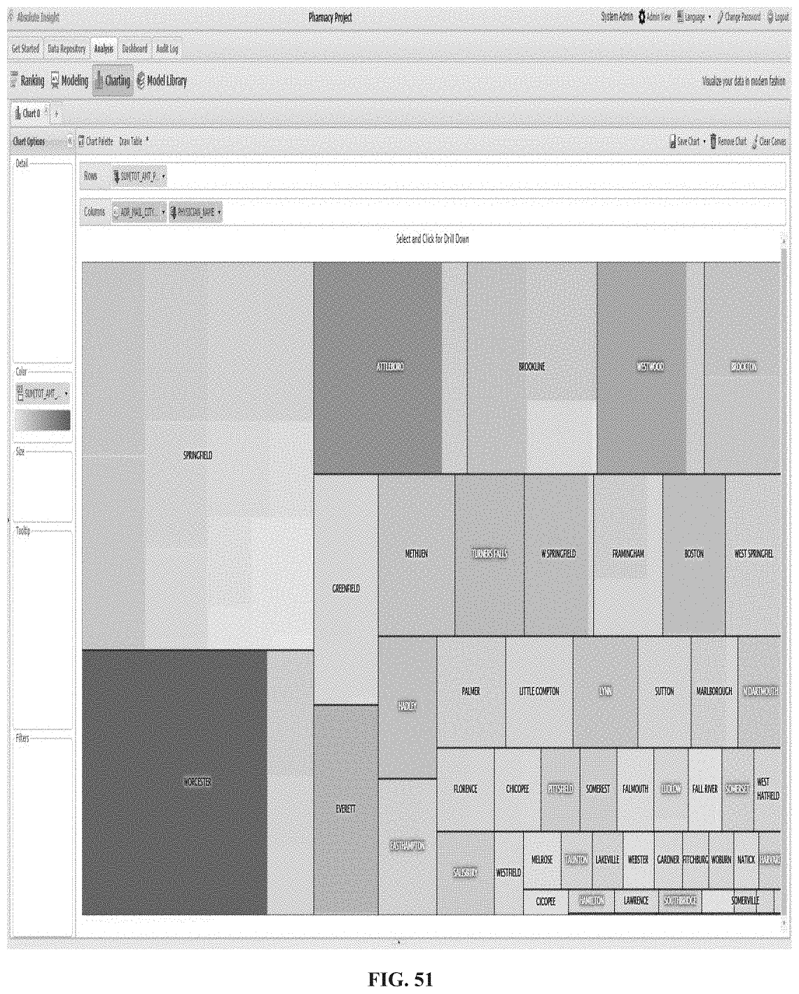

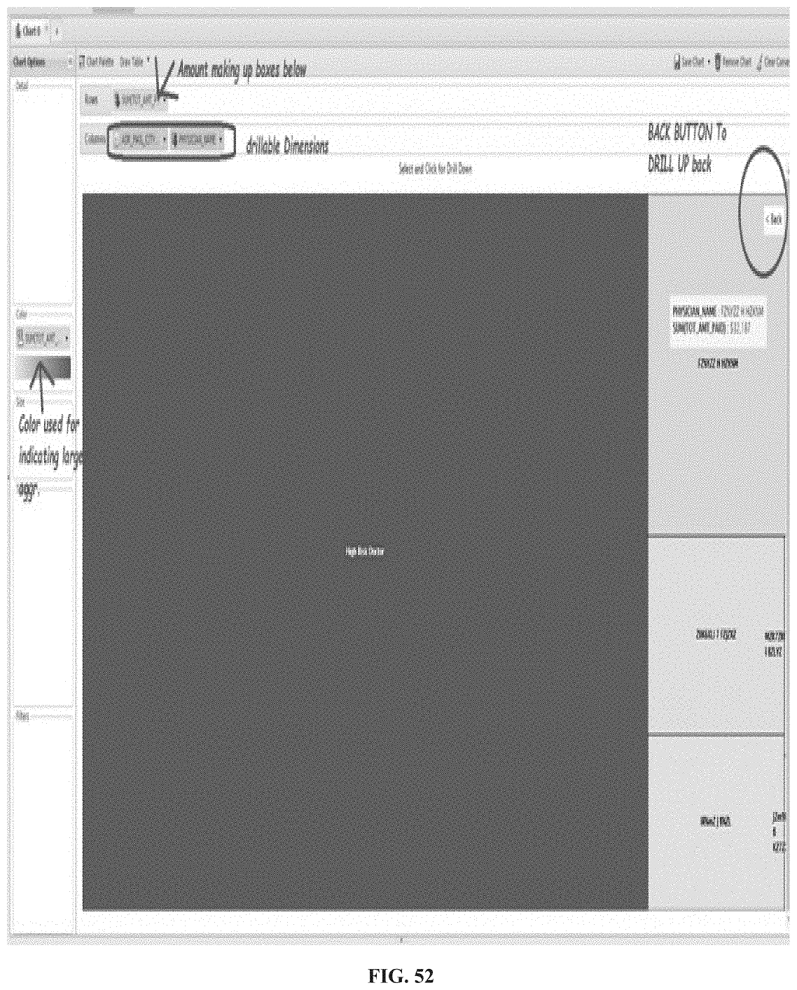

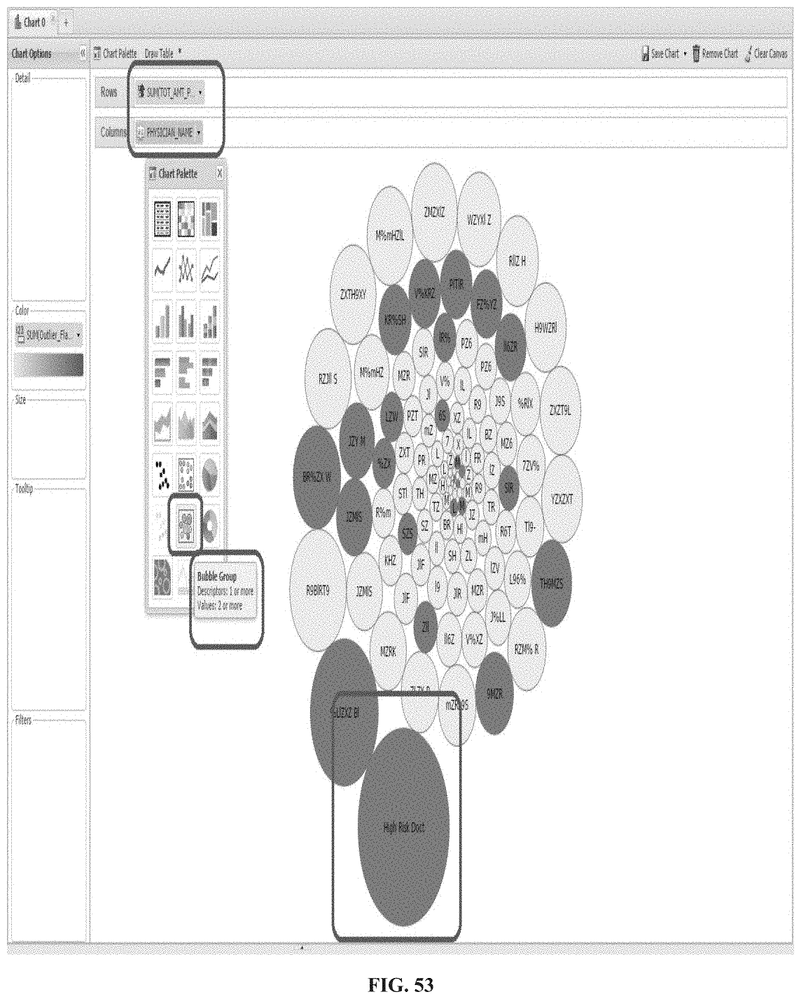

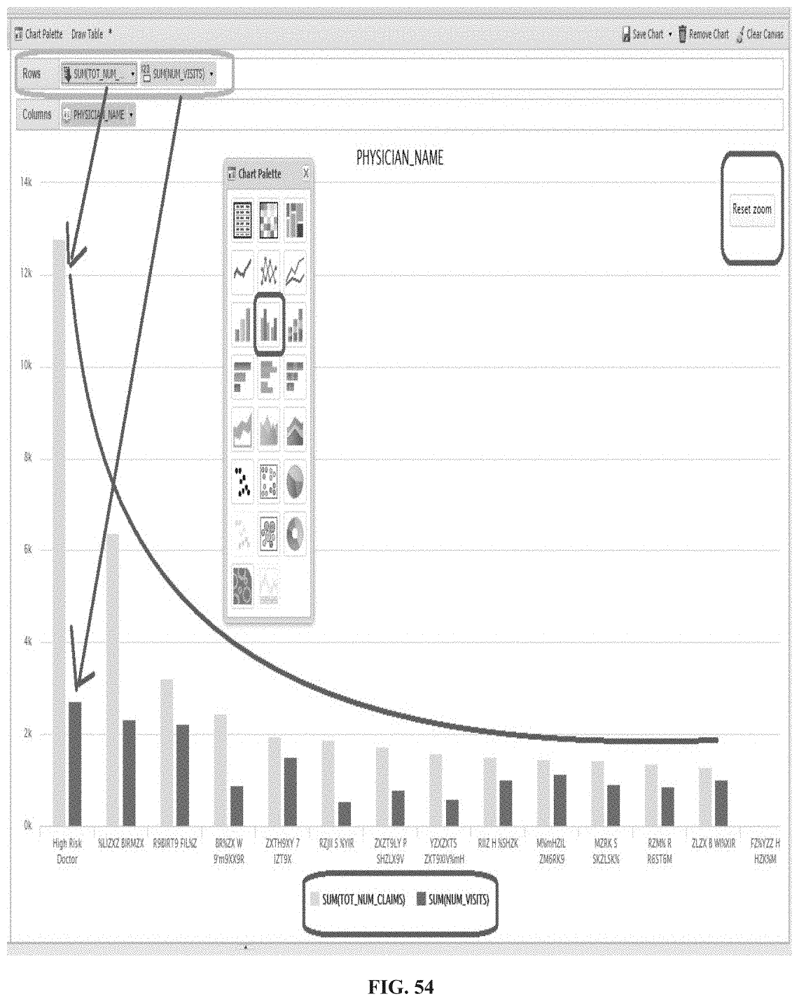

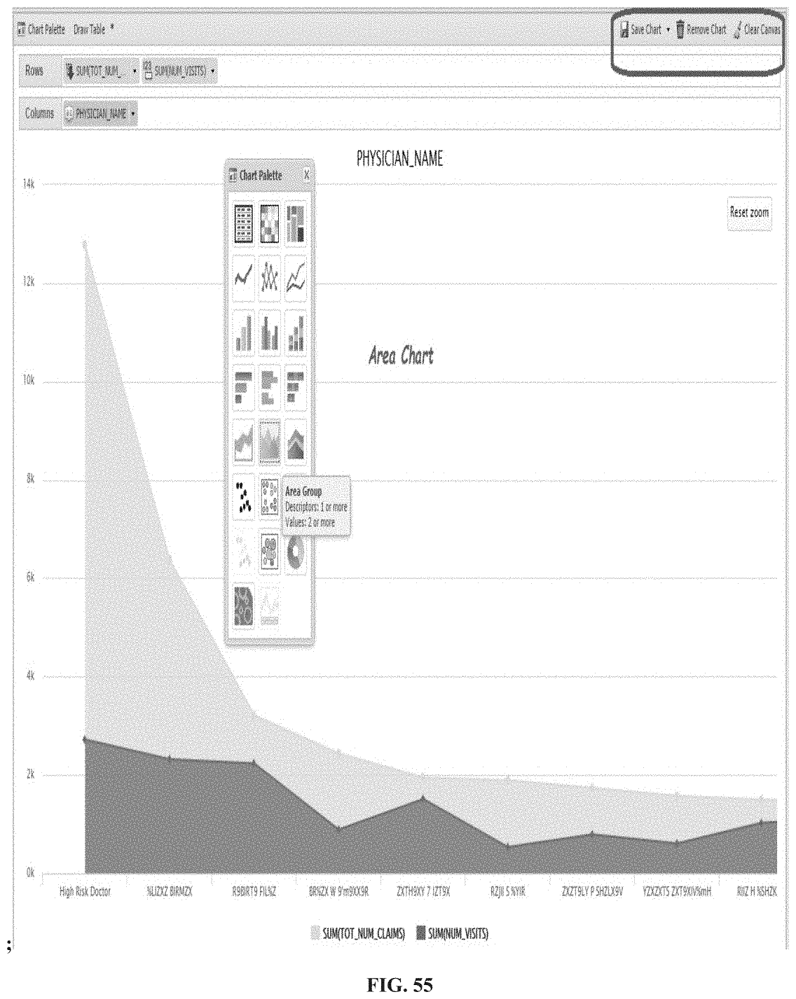

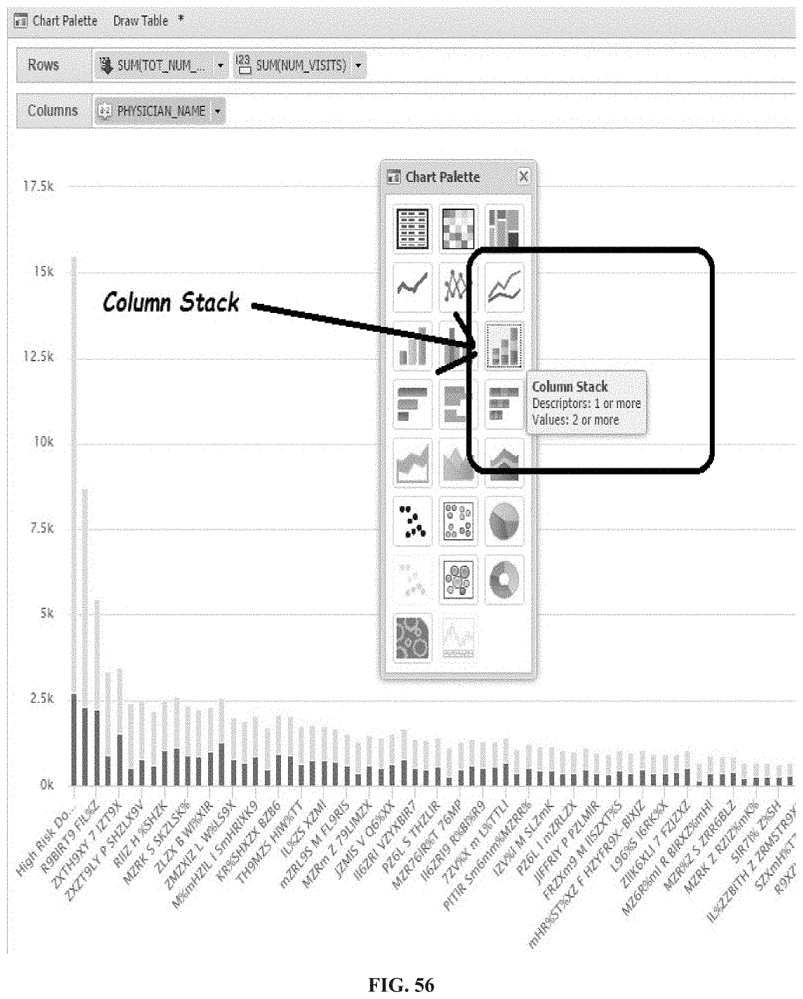

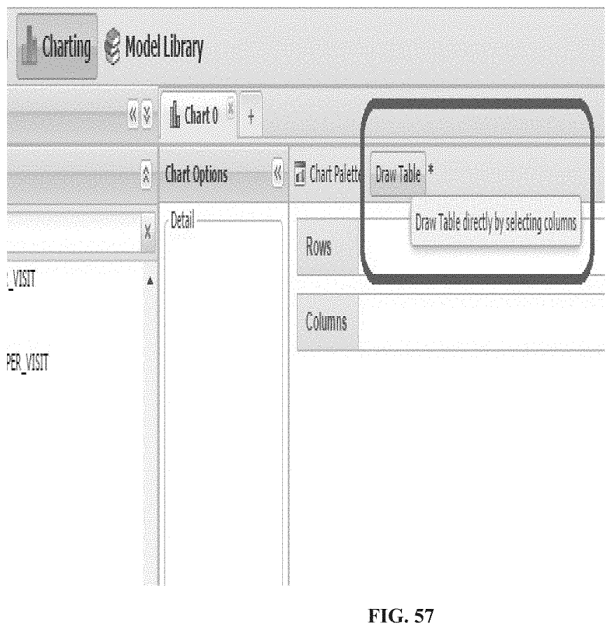

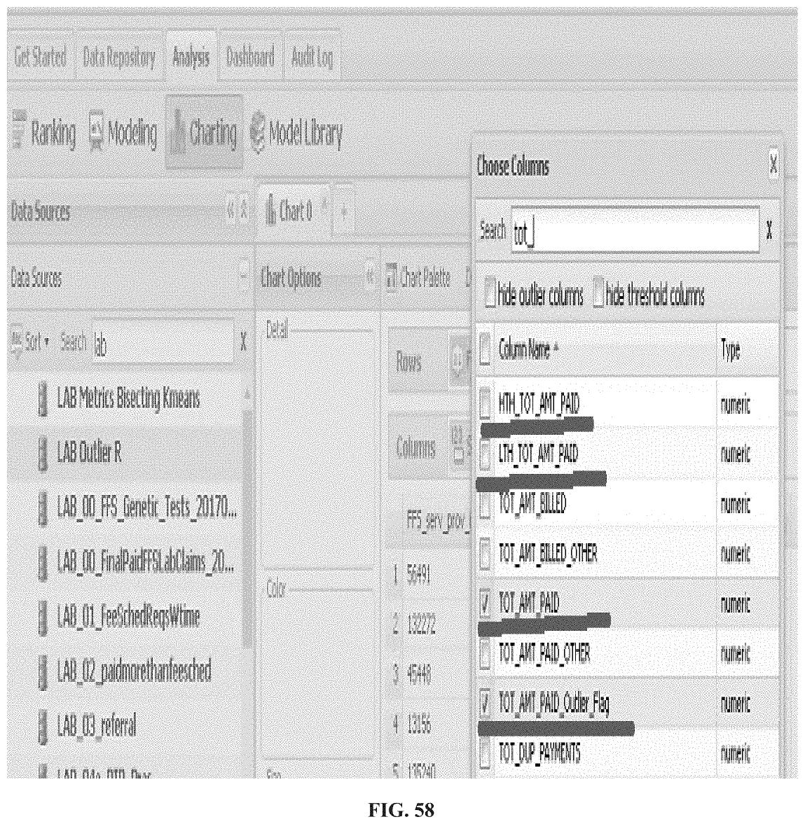

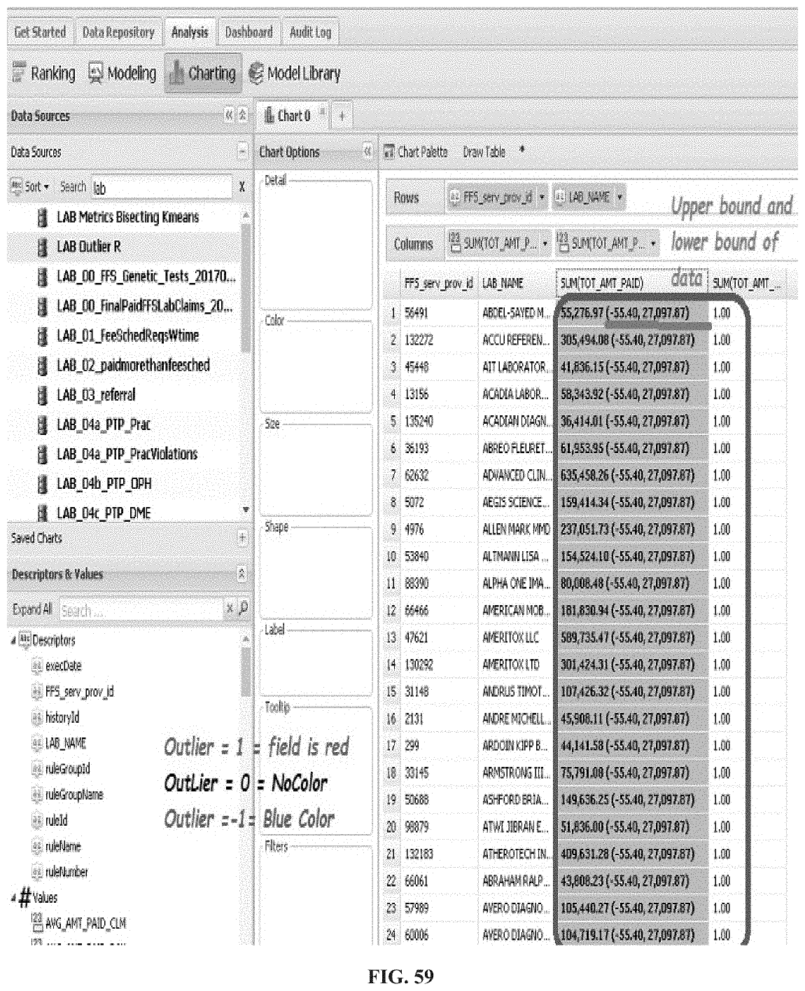

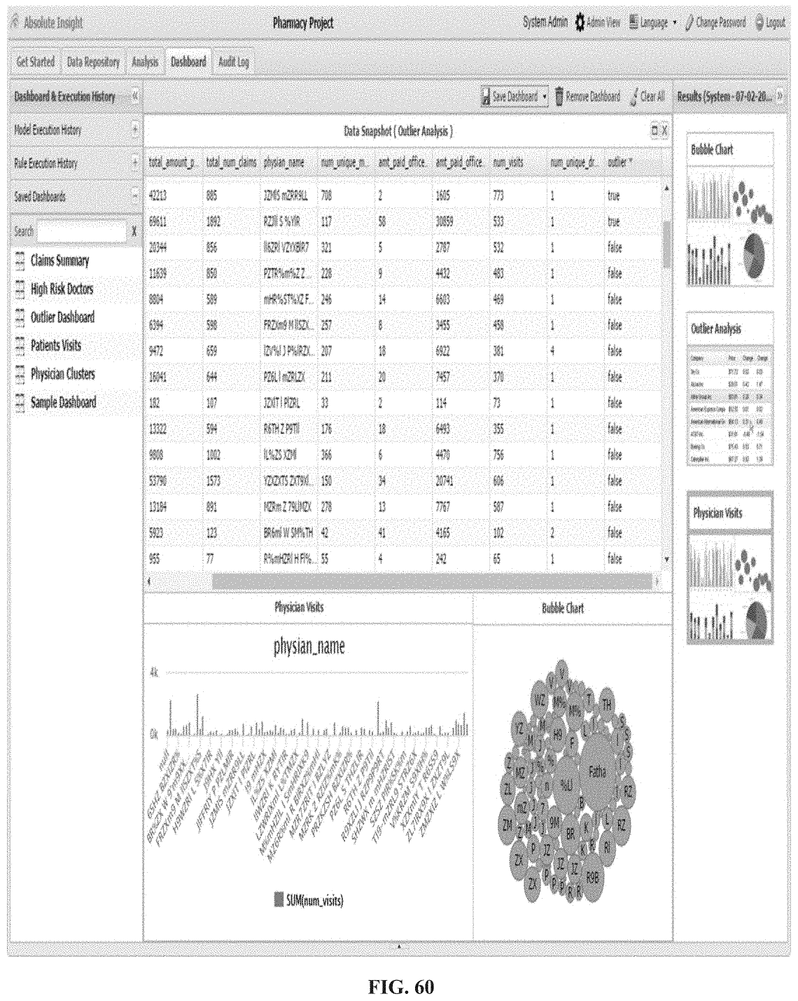

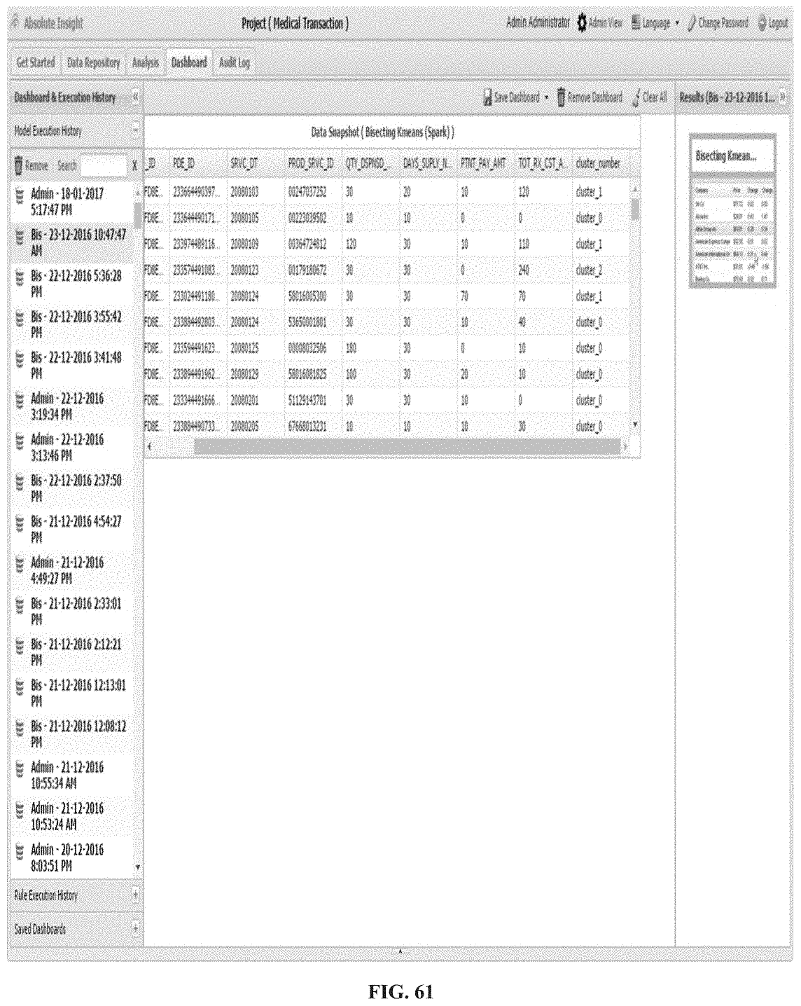

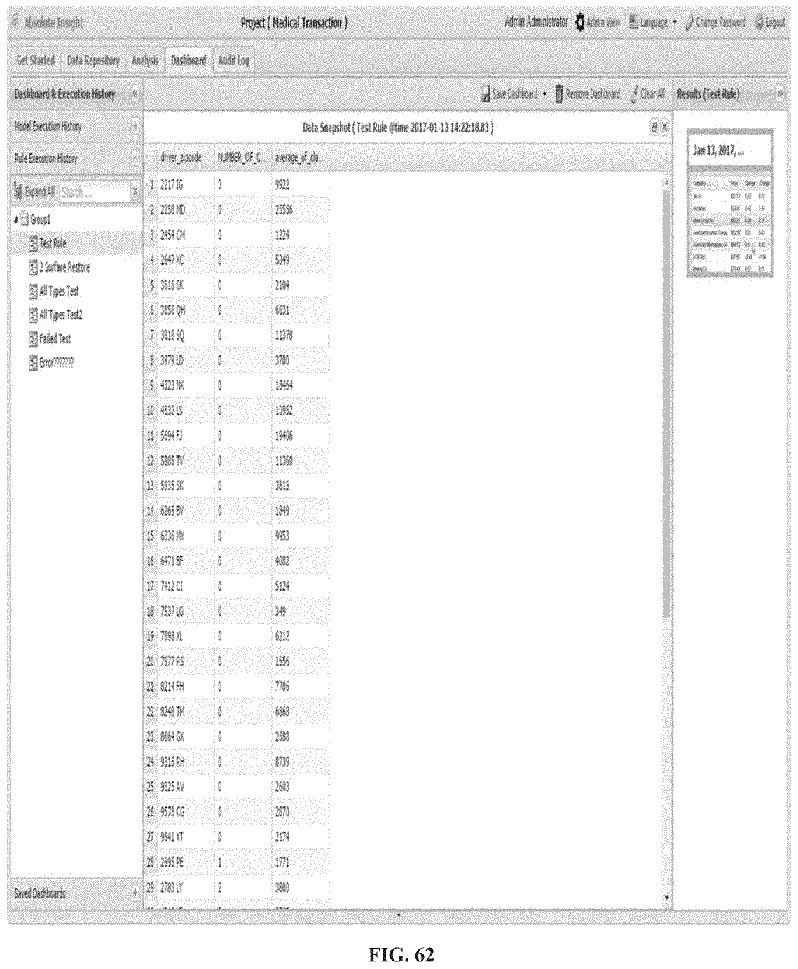

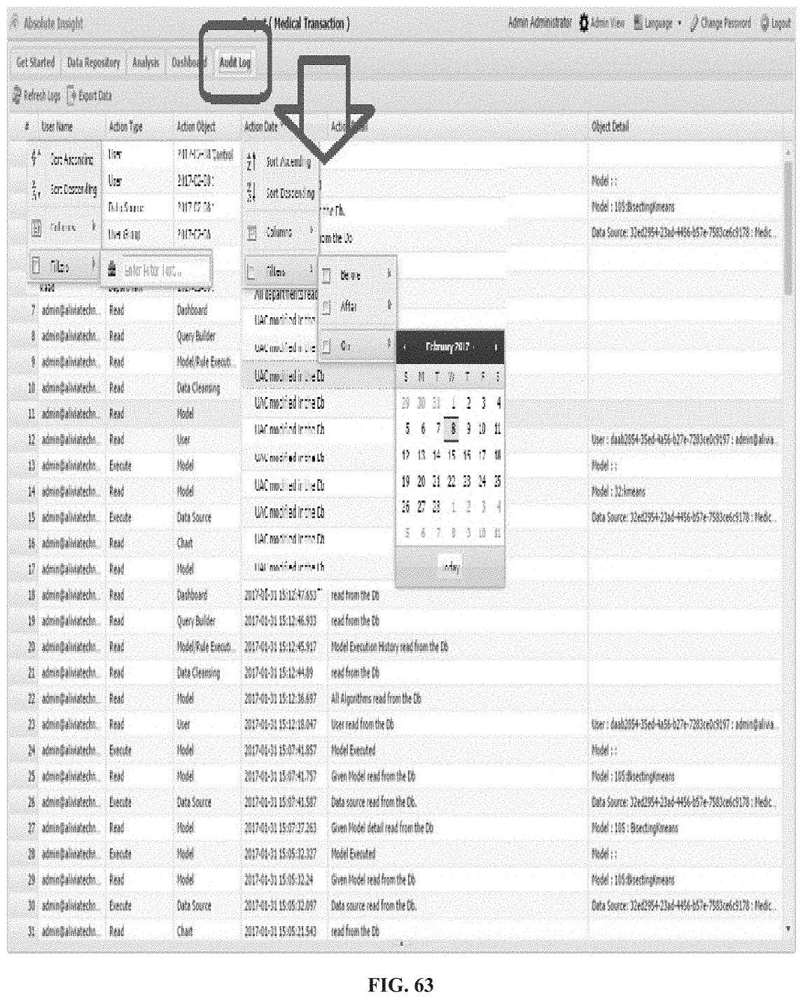

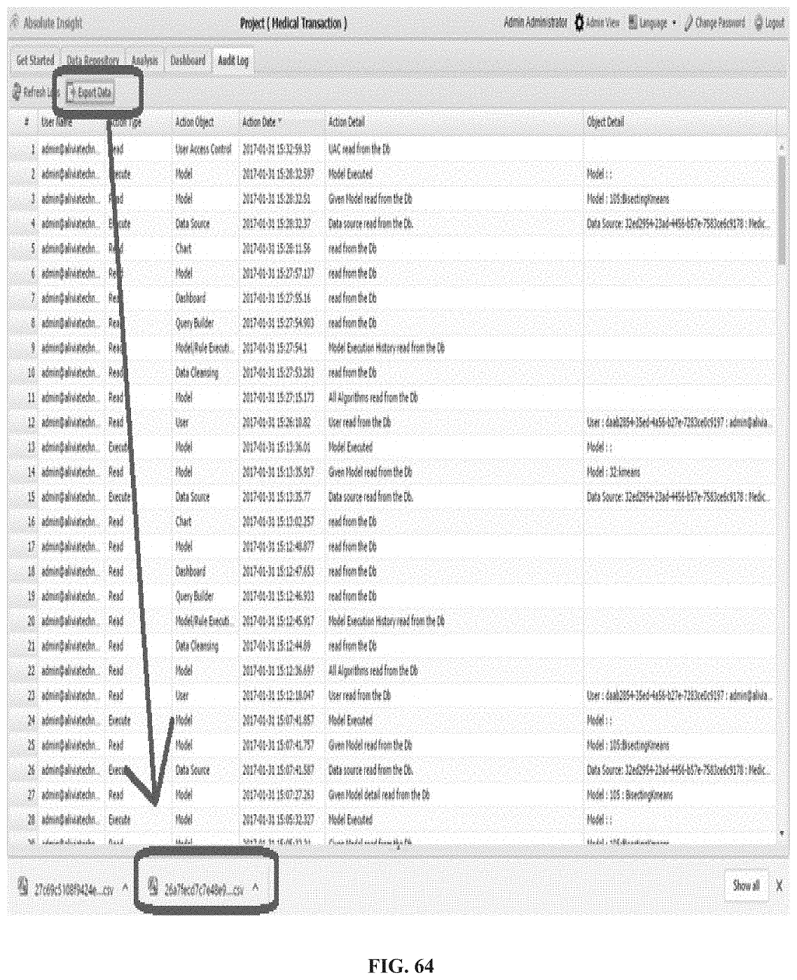

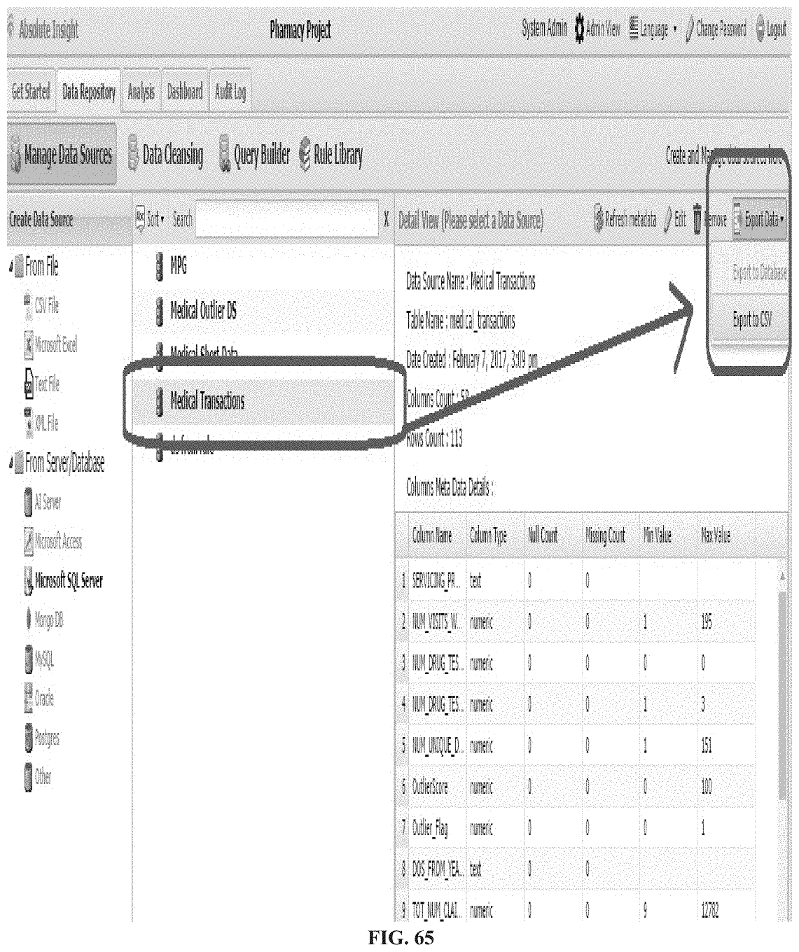

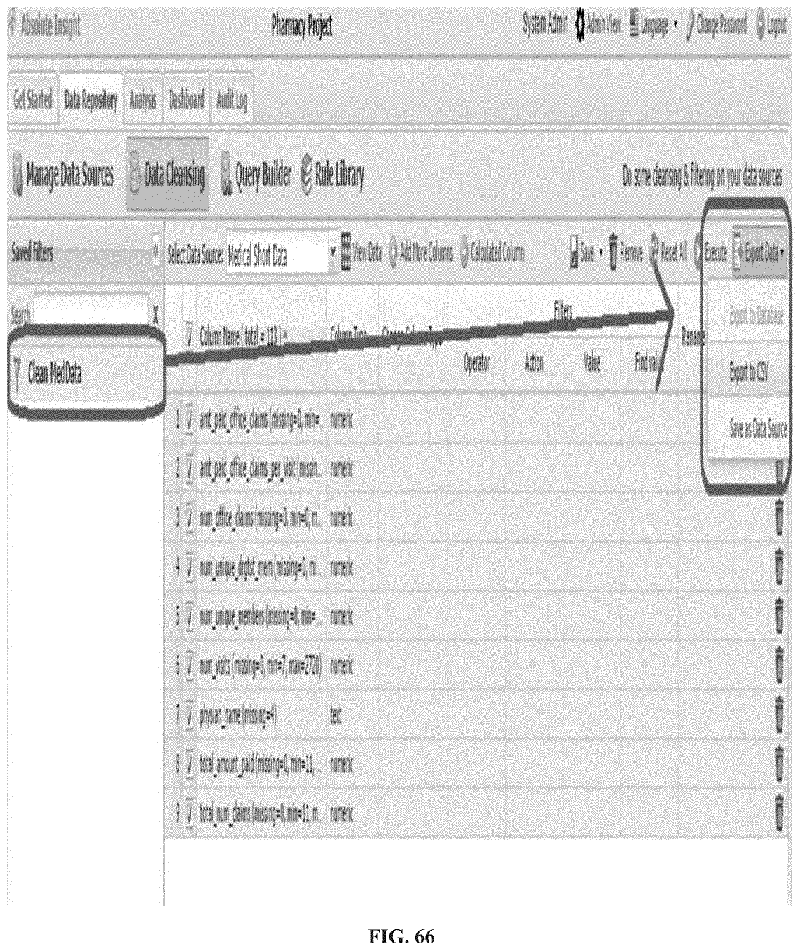

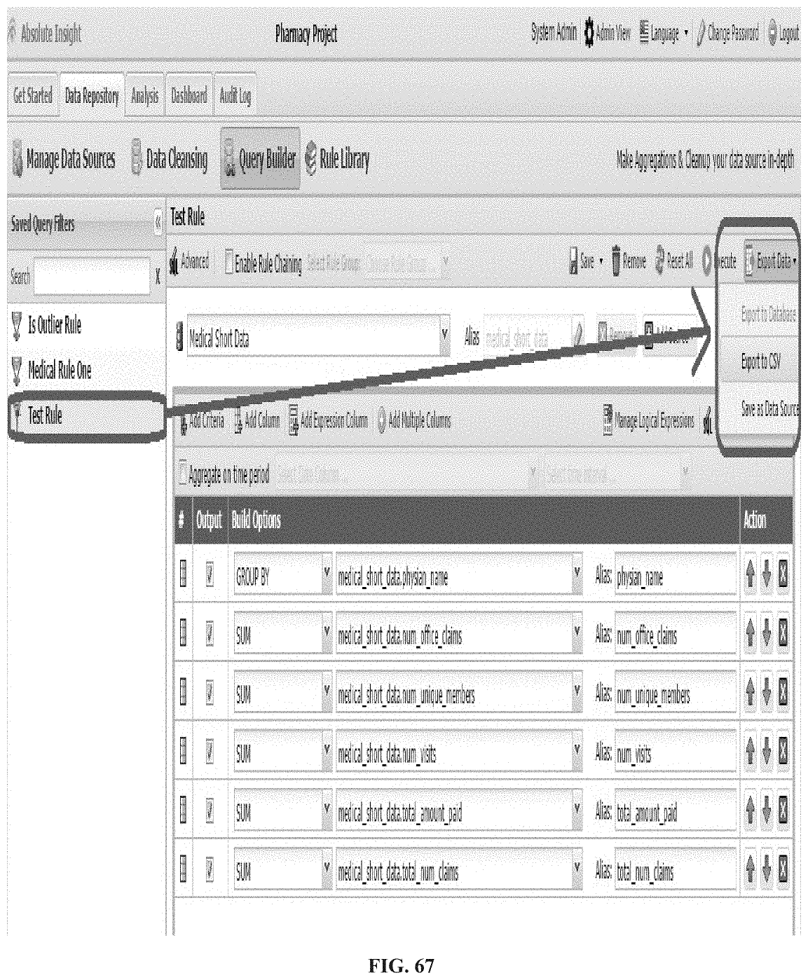

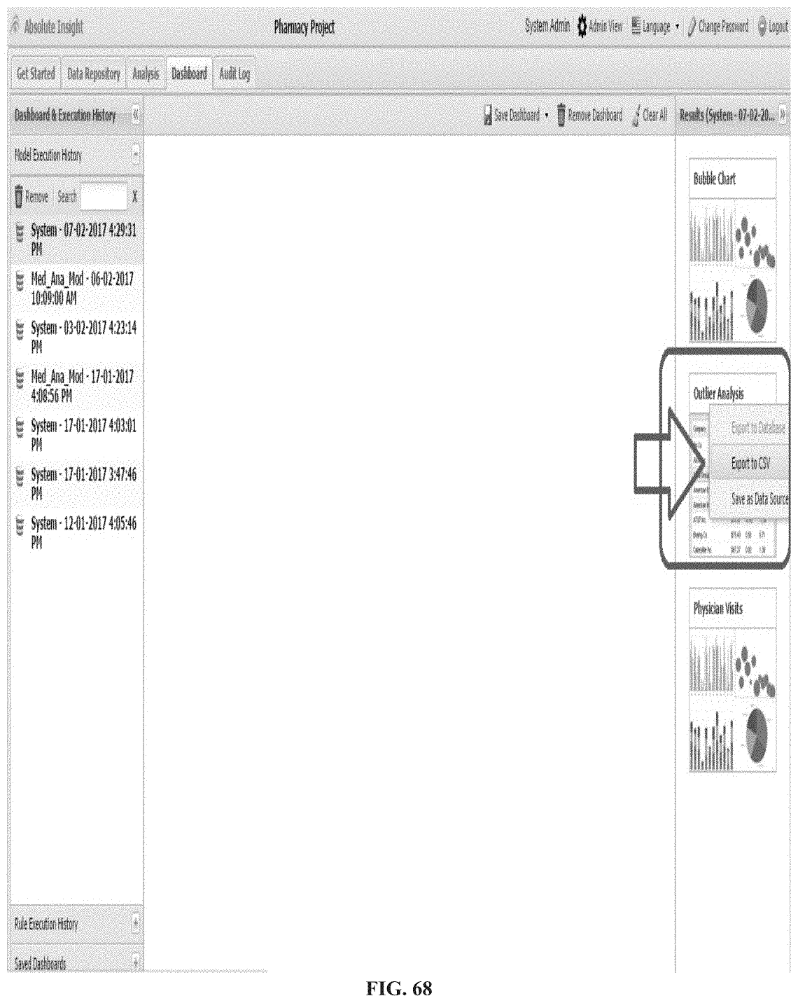

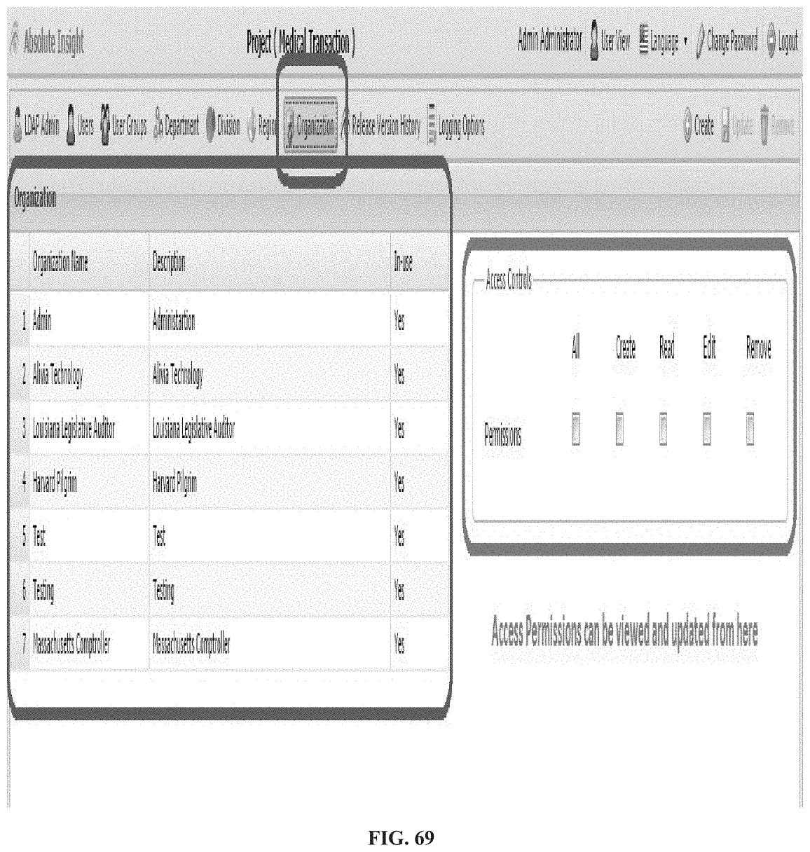

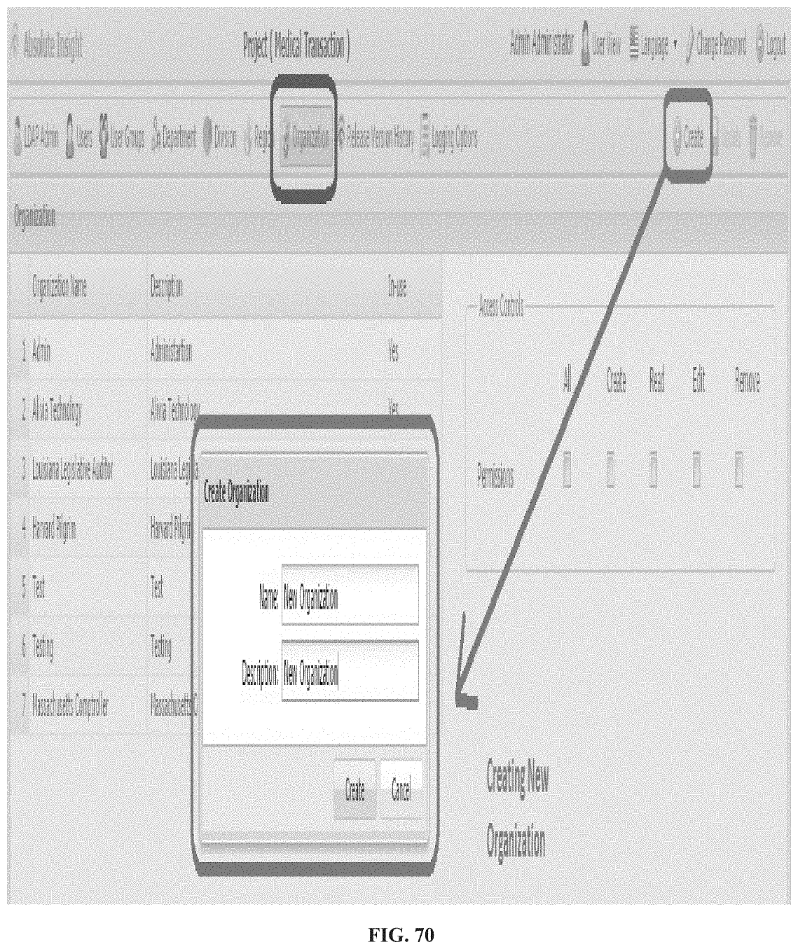

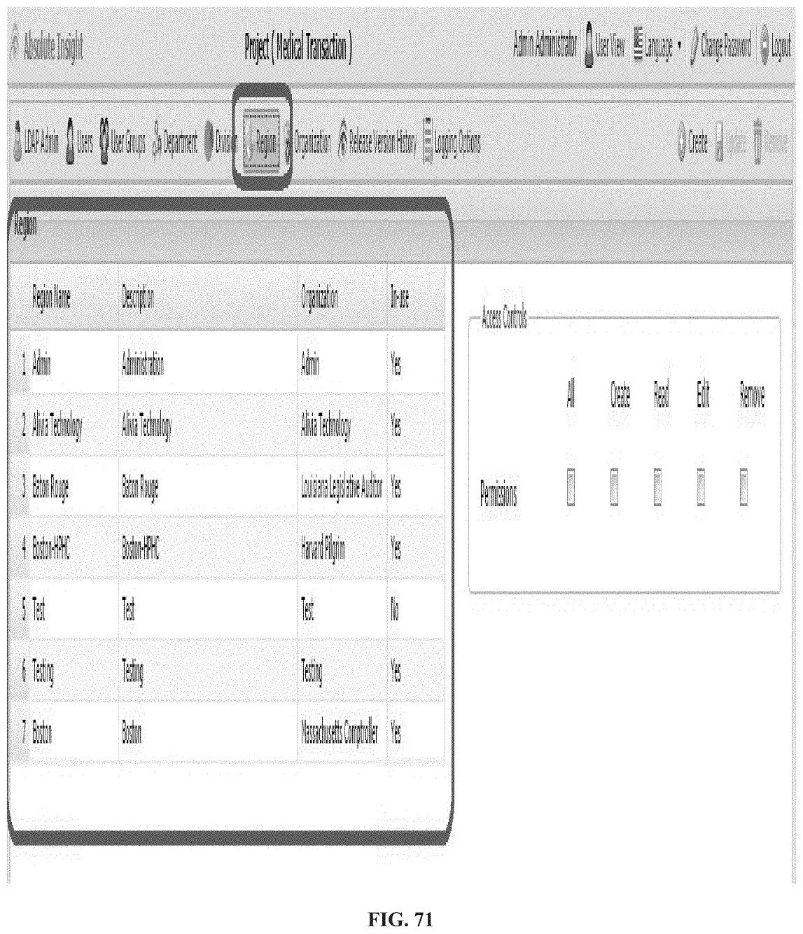

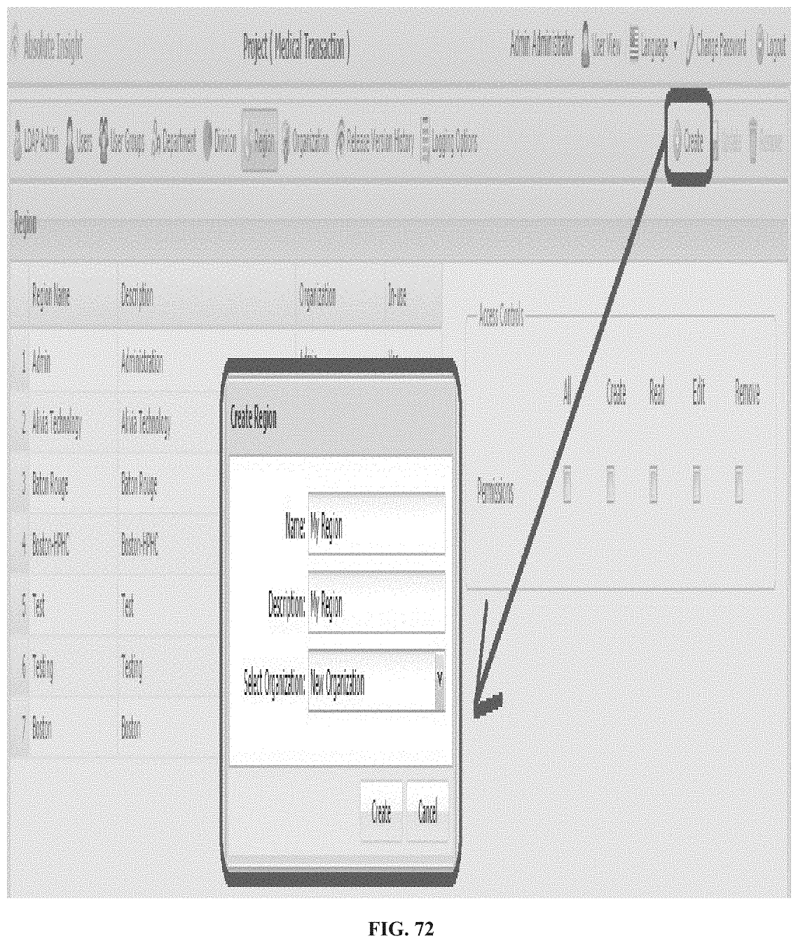

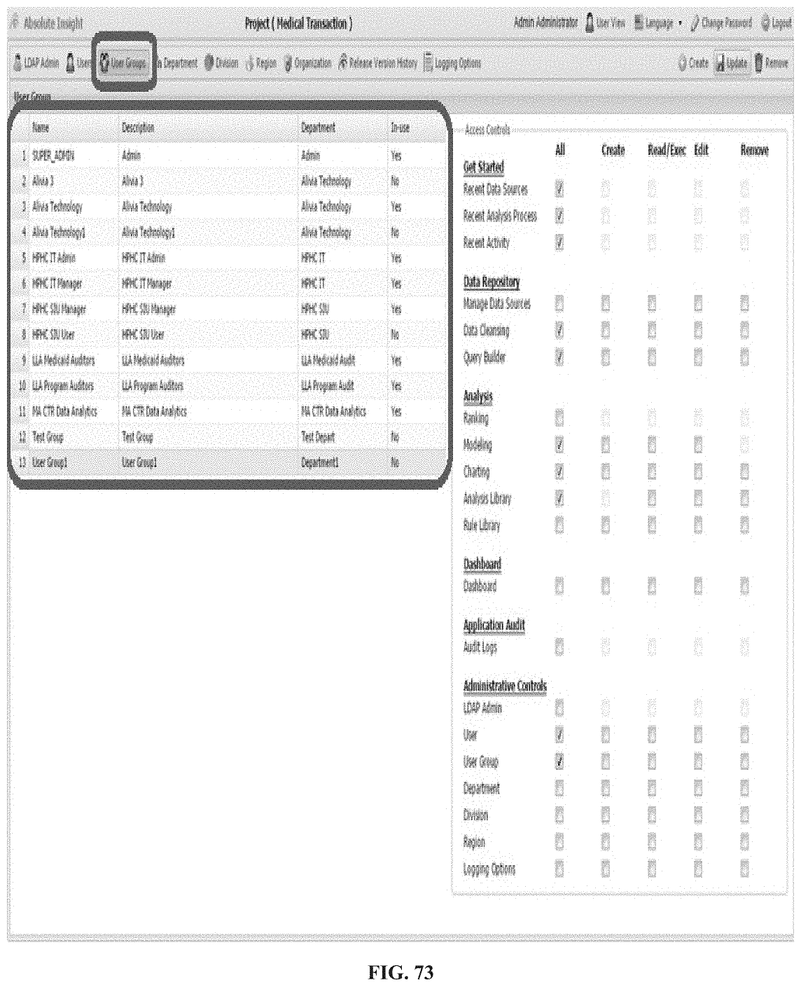

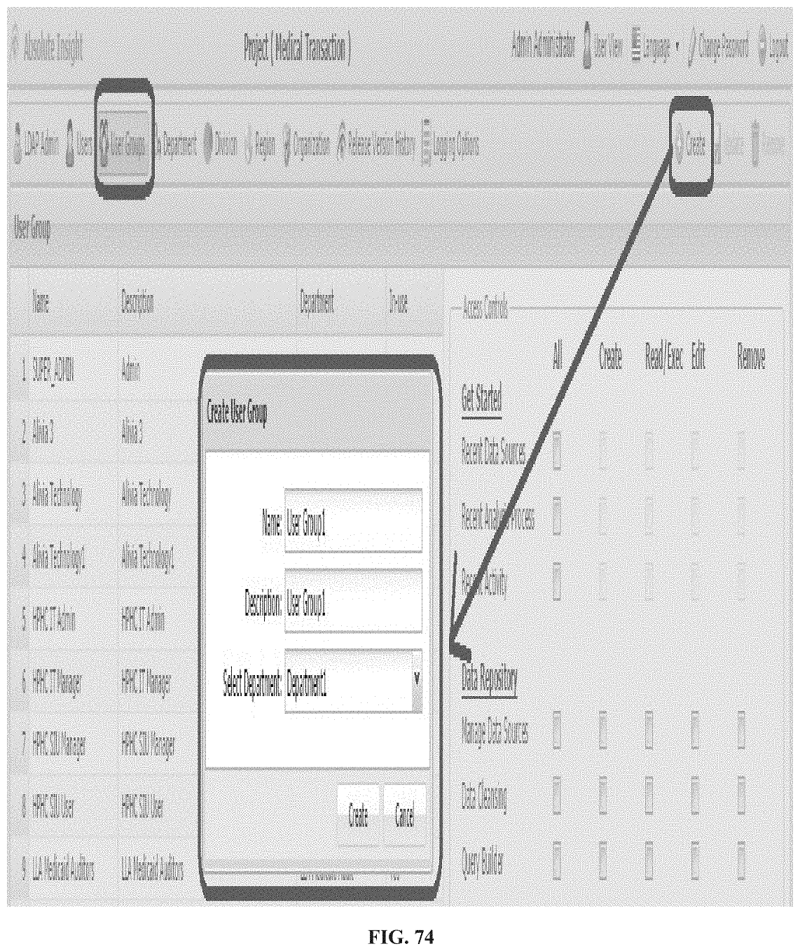

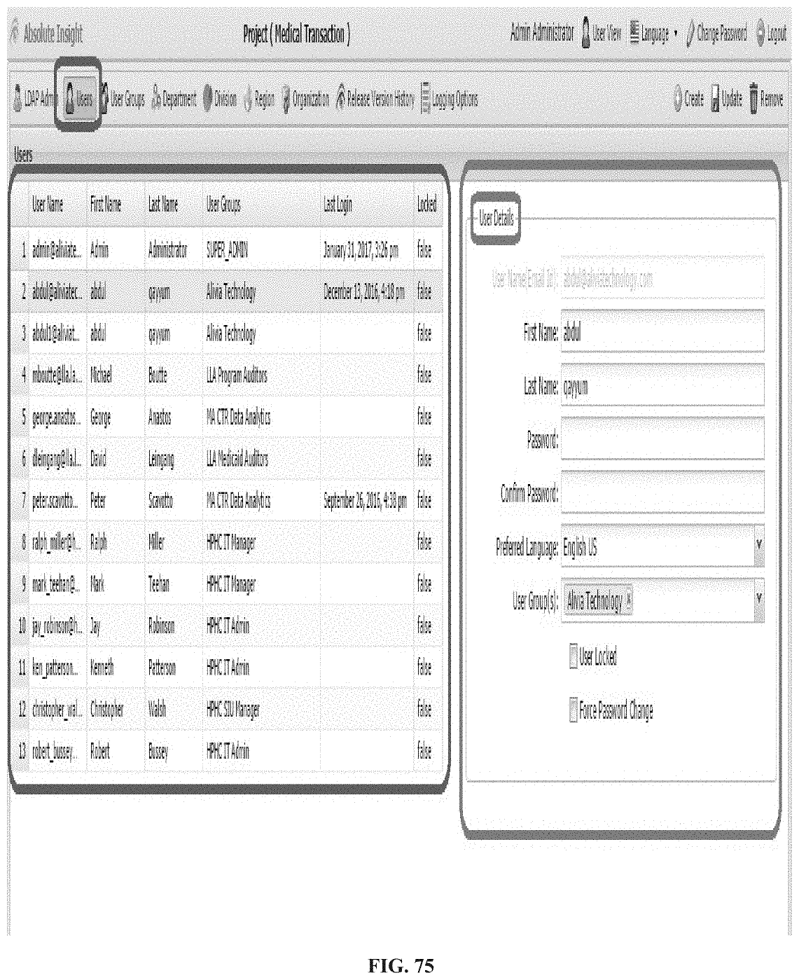

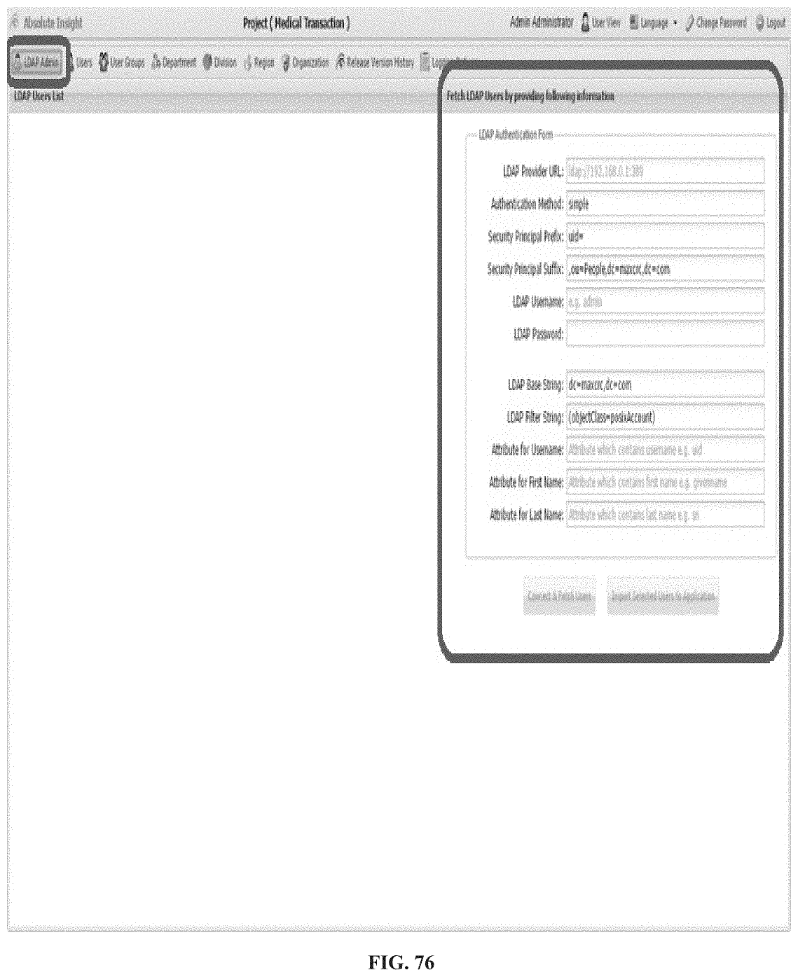

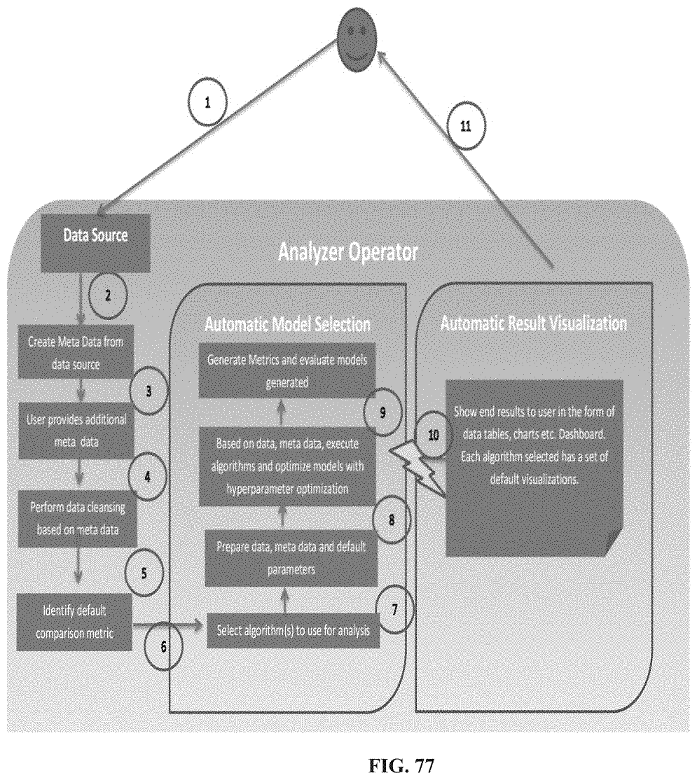

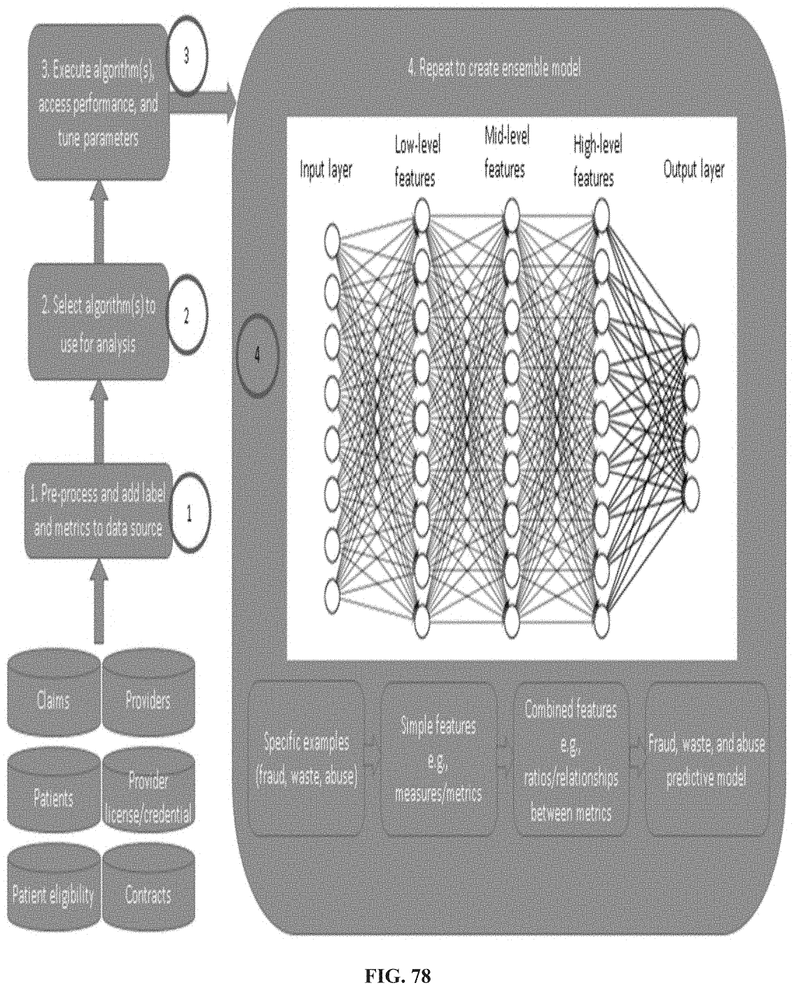

FIG. 21 is a sample annotated screen showing a portion of the sample screen of FIG. 20 highlighting a first set of controls. The following is a brief description of the various items highlighted in FIGS. 21:
Item 1 displays the Title of the currently opened Query Filter.
Item 2 allows the user to Save Query Builder configurations as a Query Filter.
Item 3 allows the user to Remove the currently opened Query Filter.
Item 4 allows the user to Reset the Editor pane to a blank state.
Item 5 allows the user to Execute the current configurations and see results in a snapshot grid.
Item 6 allows the user to Export the execution result of the query filter configuration in multiple formats such as CSV or Data Source.

FIG. 22 is a sample annotated screen showing a portion of the sample screen of FIG. 20 highlighting a second set of controls. The following is a brief description of the various items highlighted in FIG. 22:
Item 1 Allows switching between advance query editing view and interface driven view.
Item 2 allows the user to enable Rule chaining i.e. using results of one rule-filter to create new rule. It will be further explained in upcoming sections.
Item 3 allows the user to Select a data source on which you want to create query filter.
Item 4 allows the user to provide an Alias name for the selected data source.
Item 5 allows the user to Remove the data source if there are multiple data sources selected.
Item 6 allows the user to Add more data sources to create joins and complex query filters.
Item 7 allows the user to open custom query manager where the user can create queries to use in the current query filter; custom queries can be used in a specific way while creating query filter configurations.
Item 8 allows the user to open logical expressions manager, which allows the user to create logical and mathematical expressions based on multiple columns; if those expressions are used in a query filter, then they will result in adding one or more new resultant columns based on the expression.
Item 9 Allows the user to add multiple columns from the selected data source quickly.
Item 10 allows the user to aggregate data on certain time periods e.g. Yearly, Quarterly, Monthly, Daily.

FIG. 23 is a sample annotated screen showing a popup box such as when the “Add Multiple Columns” button is selected in FIG. 22. The following is a brief description of the various items highlighted in FIG. 23:
Item 1 allows the user to add a criteria row into the editor, which allows filtering of data source results.
Item 2 allows the user to add a column into the row editor, e.g., by allowing selection of one of the columns available in the selected data source to be included in execution results.
Item 3 allows the user to Add expression column, e.g., by selecting the expression created in the expression manager; the result of the expression will be included in each resultant row as a new column.
Item 4 allows the user to add multiple columns quickly by opening a popup window having a list of columns.
Item 5 allows the user to Search quickly through all columns to find and include desired columns.
Item 6 allows the user to Uncheck to exclude a given column from processing and from being included in the execution results.
Item 7 allows the user to remove an added row from the editor.
Item 8 allows the user to Move an added row up and down, which affects the order of columns in the execution results, e.g., the column that comes first in the editor will be displayed as first in the execution result.

### Rule Engine

The Rule Engine is designed around Query Builder to execute a sequence of steps needed in analysis of the data, and hence to extract useful information out of huge piles of data.

The Rule Engine gives the user complete control on the execution sequence of queries, and how and where to save and show the results for visualization or further analysis. It works out of the box, so if a user does not choose the place to save results, or position queries in sequence it still does all the jobs automatically.

The Rule Engine allows users to employ existing queries or create new queries and use them as a rule inside rule groups. All rule groups are listed with their proper title and description in the Rule Library section.

Users have the ability to execute the group of rules in a pre-defined order (large play button) with a single click or run one or more rules inside the group individually (small play button).

### Rule Library

The Rule Library as introduced above shows all the previously saved rules grouped by Rule Group for easy and neat access.

Each Rule Group listed has a big play button to execute the whole rule group, but if the user expands the list of rules inside them they can further control execution of each rule manually by clicking on the small play button which is shown beside each rule.

FIG. 24 shows a portion of a sample annotated screen displaying two rule groups.

FIG. 25 shows an expanded version of the sample screen of FIG. 24 with further annotations.

### Example Usage

The user can create a set of queries in the query builder to filter, manage, transform, and query data, for example, as depicted in the sample annotated screen shown in FIG. 26. After creating each query, the user can save it using the “Save As New Rule” button, for example, as depicted in the sample annotated screen shown in FIG. 27.

A query which is used as a rule in some rule group is preferably shown with an icon different from the icons of a standard query, for example, as depicted in the sample annotated screen shown in FIG. 28.

FIG. 29 shows a sample annotated screen that allows the user to configure a rule.

### Rule Snippet

A rule snippet is a rule that cannot be executed independently. It can only be used as a chained rule inside Query Builder while creating rules. The user can also mark an incomplete rule as snippet, so it cannot be executed, otherwise it will give errors or unwanted results.

### How to Create a Rule Snippet

Creating a rule snippet is as easy as creating a normal rule except marking it as a snippet.

In the Query Builder, when the user wants to save a Query Builder item as a rule snippet, the user goes to “Advance Options” and then checks the “Rule Snippet” checkbox to true, for example, as depicted in the sample annotated screen shown in FIG. 30.

As depicted in the sample annotated screen shown in FIG. 31, a rule snippet is available but cannot be executed.

The snippet rule can now be used in Rule Chaining, for example, as depicted in the sample annotated screen shown in FIG. 32.

In advance Sql Mode, the user can use a rule snippet wherever by just referring to the snippet using the following syntax:

Syntax: (#ruletable<<rule-name>>#)

An example is depicted in the sample annotated screen shown in FIG. 33, i.e., (#ruletable<<Snippet Rule>>#).

The user can save this new Chained Rule as a normal Rule, for example, as depicted in the sample screen shown in FIG. 34. Because this Rule is created over a “Snippet Rule”, it first gets result from Snippet Rule execution and then it executes its own configuration on retrieved results.

The user can execute this rule directly inside Query Builder to see results immediately, for example, as depicted in the sample annotated screen shown in FIG. 35. Moreover, the user also can use the export feature to save results as a data source or into CSV document format.

The user can also execute this new Chained Rule in the Rule Library and generate results, for example, as depicted in the sample annotated screen shown in FIG. 36.

### Analysis

The Analysis Module is specially designed to audit, investigate, and find hidden patterns in large amounts of data. It equips the user with the ability to identify patterns in data in just few clicks, and with a list of operators and templates which can help identify fraud, waste or abuse by few drag-and-drops.

In addition to carrying out various analyses, top of the shelf visualization tools allow plotting data, including results, to make them more meaningful, presentable and convincing. The visualizations can further be integrated into dashboards to make full investigation/audit reports, for example, as depicted in the sample annotated screen shown in FIG. 37.

### Ranking

Exemplary embodiments provide a ranking capability for data preparation and manipulation. Features range from basic sorting, filtering, and adding/removing attributes/columns, to exclusive features like creating new combined columns, re-weighting attributes, assigning ranks to each record to detect anomalies/patterns, and creating more informative views of data from the data source. In certain embodiments, each type of data (e.g., each column of data to be used in an analysis or model) is normalized to a value between 0 and 1, e.g., by assigning a value of 0 to the minimum value found among the type of data, assigning a value of 1 to the maximum value found among the type of data, and then normalizing the remaining data relative to these minimum and maximum values. In this way, each relevant column has values from 0 to 1. Values from multiple columns can then be “stacked” (e.g., added) to come up with a pseud-risk score. FIG. 38 is a sample screen showing an analysis of the Medical Transactions data source, with the results sorted by rank.

### Models

In modeling, the user can do analysis and create complex flows in Model by connecting data sources, filters, charts, dashboards, operators and algorithms. It is just easy as drag & drop items into center, connecting item's ports with each other and configuring operator parameters where necessary.

The Models Engine provides a comprehensive canvas to draw analysis visually using drag and drop features and wire up all the items together to make a flow of steps bind together to create results, the complete design can be saved as a reusable model or a template for further analysis.

### Example Usage: Models

FIG. 39 is a sample screen showing an example of a model created using drag-and-drop operations provided by the graphical user interface (GUI) of the application. On the left side of canvas, all sources, filters and other artifacts that can be used are listed. On the right side of the canvas, algorithms, operators and configuration parameters are listed. At the top of the canvas there are buttons to save, execute, or reset the canvas.

In order to create a model visually, for example, as depicted in the sample annotated screen shown in FIG. 39, the user can drag and drop a data source from the left side onto the canvas, drag and drop an algorithm operator onto the canvas, graphically interconnect the data source with the algorithm (e.g., by connecting the output of the data source icon with an input of the algorithm icon) to have the algorithm performed on the data set. In the example shown in FIG. 40, the data source Medical Transactions icon is graphically connected to the Bisecting K-Means algorithm icon and the final results are wired to the output port. The user can execute this model by clicking on the “Execute” button, and when finished, the model can be saved for repetitive usage. It should be noted that different types of blocks (e.g., data sources, algorithms, filters, etc.) may allow for multiple inputs and/or multiple outputs. Thus, for example, if the user had dragged two data sources onto the canvas and connected them to the algorithm block, then the algorithm block would operate on the two data sources. Using these drag-and-drop operations, the user can easily set up a model in which one or more data sources can be operated upon by one or more algorithms or filters (in sequence and/or in parallel), and also can include various types of visualization blocks (e.g., graphs, charts, etc.) to produce visual displays based on the output(s) of one or more other blocks. Thus, the model shown in FIG. 40 is a simple one having one data source and one operator, although more complex models can be built using a wide variety of combinations of data sources, operators, and visualization blocks.

When model execution completes, it will automatically take the user to a “Dashboard View” in order to show the execution results, for example, as depicted in the sample annotated screen shown in FIG. 41. Here, the dashboard view shows all execution results in one area and shows a result table in another area.

### Operators

Operators are a collection of artifacts, functions and algorithms that are used to create models or templates. An operator can have parameters, input ports and output ports associated with it. Input/output ports are used to connect multiple operators with each other and to the output port of the model, which will transfer data from one operator to another. When the model executes, the operator performs certain processing and actions before sending data to output port. Parameters associated with the operator can be used to control the behavior of the operator. FIG. 42 is a sample annotated screen showing various types of operators, including, in this example, a set of basic statistics operators, a set of classification and regression operators, a set of clustering operators, a set of filtering operators, a set of frequent mining operators, a set of outlier operators, and a set of R operators. Each set can include multiple operator blocks, each implementing a different algorithm. For example, the set of outlier operators may include multiple outlier operator blocks, each one implementing a different algorithm for analyzing outliers in the data (e.g., using different metrics from the data source and/or different algorithms for determining an outlier classification based on the metrics from the data source). Thus, for example, a particular doctor might be considered an outlier in one model but not considered an outlier in another model.

Parameters are settings of operator which can be seen by clicking on the operator in the center canvas. All associated parameters will be listed in the “Parameter View” in the bottom right corner in modeling. One can alter operator behavior by changing parameter values.

### Models Library

The Models Library shows all the models that are saved by the user from the modeling canvas. As depicted in the sample annotated screen shown in FIG. 43, models can be deleted if the user no longer wants them, models can be loaded into the modeling canvas in order to view or modify them, and models can also be executed directly by clicking on the play icon without loading it to modeling canvas. This feature allows executing different models concurrently.

### Charting

Charting offers a wide variety of chart types to be used against data sources. It is fully capable of displaying scatter, line, bar, bubble, area, pie, doughnut, and more plots for various descriptors and values.

The charting engine is equipped with aggregations functions, filters, sorting and all the ingredients needed to neatly prepare a meaningful visualization, the chart palette is floating and can be moved out of the view for easy canvas access while building various charts.

The charting engine is intelligent enough to decide on the fly which aggregations would be appropriate for the selected chart and if the current selection of attributes would not fit in a single chart then it creates multiple charts with a scroll bar.

To plot any chart, the user selects a data source the from top left in the charting module, for example, as depicted in the sample annotated screen shown in FIG. 44. When a data source is selected, it will display its columns/attributes in the bottom left corner and assign columns/attributes into one of the following categories: descriptors (text type columns), values (numeric type columns) and dates (columns which are stored as dates in database), as shown below.

Now the user can drag descriptors and values of their choice and drop them into the specified input slots given in the charting canvas. In one exemplary embodiment, the available inputs are Rows, Column, Detail, Color, Size, Tooltip and Filters. There is wide variety of charts supported. Some of the types are detailed below.

### Plotting a Bar Chart

FIG. 45 is a sample annotated screen that highlights that a potentially high-risk doctor has been identified easily by plotting their name in “rows” and putting “total amount paid” in “columns”; also the charting engine “color” feature is engaged to Color band “high red” the high risk doctor also in tooltip all the required information for the doctor is added to visually see on hover to see complete info about the doctor.

The user can change the type of chart by using the Chart Palette. The Chart Palette offers a variety of charts to be draw for the given inputs, and it automatically enables the chart types, which would function given the provided inputs. The number of required descriptors and values for each chart can be seen in the tooltip by taking mouse over to the chart icons.

### Plotting Pie Charts

A potentially high-risk provider can be found by simply plotting TOT_AMT_PAID against PHYSICIAN_NAME using a pie chart, for example, as depicted in the sample annotated screen shown in FIG. 46. Bigger areas/cones in the pie indicate outlying entities. In the tooltip one can see the total amount that has been paid to this physician, for example, as depicted in the sample screen shown in FIG. 47.

### Using Filters in Charting

If the plotted data is too large or the user wants to visualize only meaningful data (e.g., fitting given criteria), then descriptors and values can be dropped into the “Filter” input slot, for example, as depicted in the sample annotated screen shown in FIG. 48. This allows filtering and selecting only data that are needed for visualization. Here, the search box enables looking up values.

### Using Color Feature in Charting

An example of a bar chart with gradient color is shown in the sample annotated screen shown in FIG. 49. Colors can also be customized: here darker red color of bars indicates higher amounts paid to the corresponding entities.

### Creating Scatter Plot in Charting

FIG. 50 is a sample annotated screen showing a scatter plot using the Detail, Color and Size input slots. Drag and drop “TOT_AMT_PAID” and “TOT_NUM_CLMS” to the Rows and Columns slots, respectively, then OutlierScore- to Color, and NUM VISITS to size, and you can now notice that size of a circle indicates the number of visits for each physician while color denotes a degree of “outlyingness.” Here the total number of claims is plotted against the amount paid to each provider. Therefore, the scatter chart can identify anomalies from different perspectives. The X axis can be used to find entities that are scoring higher on amounts paid, while the higher the dot is, the more paid claims the physician has. On the other hand, darker color highlights entities which are flagged as outliers by an analysis algorithm. Finally, the point/circle size shows which provider has the higher number of visits.

Creating Drill Down-Tree Map plot in Charting

Tree maps display hierarchical data by using nested rectangles, that is, smaller rectangles within a larger rectangle. The user can drill down in the data, and the theoretical number of levels is almost unlimited. Tree maps are primarily used with values which can be aggregated.

FIG. 51 is a sample screen of a Tree Map showing physicians grouped by their geographical location and locations with the highest propability of containing outliers.

This chart is easy to create: the user can just drag and drop text type descriptors (dimensions of cube) in columns drop values (measures) in the rows. The user can add multiple descriptors in chain to create a dynamic drillable chart as above. For example, in the sample screen shown in FIG. 51, if the user clicks on “Worcester”, then the application will drill down to explore all Worcester physicians.

FIG. 52 is a sample annotated screen showing a back button in blue circle above, which allows the user to drill back up.

### Creating Bubble Group in Charting

The user can select any Descriptor in Columns and drag and drop any measure against it in Rows, for example, as depicted in the sample annotated screen shown in FIG. 53. Then, for example, the user can drag and drop Physician name and Total amount paid in both to create a bubble chart, and the application can sort it on the total amount paid to see the high risk doctor on top.

Also, in order to create any chart, if users hover over the chart palette on the chart, it will give information about that chart and how to create it. In the case below it shows that at least one descriptor and 2 or more values are needed to draw a bubble chart.

In FIG. 53, the chart is created in a tab called “Chart 0” and there is a small (+) sign next to it that indicates that the user can create, load or save multiple charts in parallel as well.

Creating Grouped Bar chart

FIG. 54 is a sample annotated screen showing a grouped bar chart with more than one set of values that the user wants to see side by side for physicians like total number of visits and total number of office visits. In order to produce this chart, the user typically would drag & drop both total number of visits and total number of office visits to Rows and physician name to column and sort it on one of the measures.

The user also can use zoom by just dragging the mouse while holding left mouse key into an area of the chart. Once the user has zoomed-in on an area of the chart, the user can zoom out by selecting the ‘Reset Zoom’ button on the top right as highlighted in FIG. 54.

Line charts, Grouped Line charts, Bar Graphs, Grouped Bar Graphs, stacked Bar Graphs, area charts, and grouped line charts all work in a similar fashion. For example, FIG. 55 shows a sample annotated screen of an area chart corresponding to the grouped bar chart of FIG. 54.

Note that the user will always have an option to save chart, remove chart or clear the canvas totally.

Note that when the user selects a chart from the charting palette, it is highlighted in the palette and it also shows the required ingredients to make that chart and the chart name, for example, as depicted in the sample annotated screen shown in FIG. 56.

### DRAW Colored TABLE

In order to draw a table, the user can click on “Draw Table” on the top center in charting tab, for example, as depicted in the sample annotated screen shown in FIG. 57.

Upon clicking on “Draw Table,” a “Choose Columns” pop-up screen appears to allow the user to select the columns to use for the table, for example, as depicted in the sample annotated screen shown in FIG. 58.

For the table of FIG. 58, the user has to select a Value (numeric column) that has 3 columns: LTH_column_name, HTH_COLUMN_NAME, and COLUMN_NAME_Outlier_Flag, in above case TOT_AMT_PAID is the target column as it has all the ingredients to create the table, so the user can select it along with its outlier column just to see if it is correct.

FIG. 59 shows a sample annotated screen with resulting information from the selections in FIG. 58. Here, the sorted information includes upper and lower bounds on the data as well as color-coding based on the outlier flag, e.g., the field color is red to indicate that the outlier flag value is 1; data having an outlier flag of −1 might be shown in blue while data having an outlier flag of 0 might be shown without color. Generally speaking, an outlier is an entity that is significantly different than the norm for a given set of performance metrics (e.g., if doctors typically submit two claims per patient on average but one particular doctor typically submits 5 claims per patient on average). In certain exemplary embodiments, outliers may be identified using a Robust Principal Component Analysis (ROBPCA) method as known in the art.

### Dashboards

Dashboard is used to present analysis work done on data and final results. It also holds Model execution results as well as rule execution results, which can also be used to make a dashboard. Dashboards can be saved as well.

### Dashboards Usage

In order to add a grid or a chart to a dashboard, the user can select any Model/Rule execution item from “Dashboard & Execution History” (left side) of Dashboard, for example, as depicted in the sample annotated screen shown in FIG. 60. All related results of that particular item will be displayed on right side of dashboard, for example, as depicted in FIG. 60. The user can double-click on any item which is grid or chart, and it will open a box window in the center of the dashboard. Box window can be resized and dragged anywhere in the center area. This way, all items can be positioned to a suitable location.

FIG. 61 shows a sample screen with Model Execution History information.

FIG. 62 shows a sample screen with Rule Execution Results information.

### Logging Mechanism

Log messages are useful for a various reasons. For example, log management can log the entire read, write, create and delete operations on data and also can keep track of user logins. Security experts, system administrators and managers can view and track all of the log messages coming from the server. The user can filter the messages and sort messages of a specific category to group similar messages together. Lot information in log messages can be discovered with a powerful search option.

### Usage of Audit Log

All operation logs can tell which user has logged in to application at what time, and also can tell what items were created or removed from the application and what items were executed and at what time particular operations were performed along with useful detail information. For example, if the user creates a data source, it will be marked as a create action on a data source action object with a timestamp. If the user executes an algorithm, then it will be marked as an execute operation. FIG. 63 is a sample annotated screen with an audit log. Also, the user will have an option to export these logs into a csv formatted document, for example, as depicted in the sample annotated screen shown in FIG. 64.

### Exporting as CSV

The user can export data into a standard CSV document, which is a simple, flat and human readable format. CSV is understood by almost every piece of software on the planet. There are various places in the application where the user can do CSV data export. For example, FIG. 65 is a sample annotated screen for exporting a Data Source into CSV from the Manage Data Sources tab; FIG. 66 is a sample annotated screen for exporting cleansing filter results data into CSV from the Data Cleansing tab; FIG. 67 is a sample annotated screen for exporting query filter results data into CSV from the Query Builder tab; and FIG. 68 is a sample annotated screen for exporting Dashboard results data into CSV.

### Security Module

The user access control mechanism in the Absolute Insight application has hierarchical structure. Each level contained in the hierarchy can have different control permissions. These permissions have a pattern of effectiveness from top to down in the hierarchy, e.g., granted permissions in a lower level in the hierarchy can be denied by an upper level if those permissions are not assigned in the upper level. The following summarizes the list of security levels in order of effectiveness in the security hierarchy:

Organization>Region>Division>Department>User Group>User

### Levels: Organization, Region, Division, Department

Organization is the top most access control security level of the Absolute Insight application's user interface. Access control permissions will override the permissions of its subsequent level i.e., Organization will override permissions of assigned Region of an organization.

### Example Usage

Each access control level contains the following capabilities (Entity represents any of Organization, Region, Division or Department)
Lists all available entities
View assigned permissions by selecting each entity
Update permissions of any entity
Create new access control entities
Remove any access control entity

An entity that is assigned to one or more subsequent levels (like Region assigned to Divisions) cannot be removed and will be marked as “Locked” until it is no longer assigned to any subsequent level.

FIG. 69 shows a sample annotated screen with Organization access permissions, which can be viewed and updated.

FIG. 70 shows a sample annotated screen for creating a new Organization with access permissions.

FIG. 71 shows a sample annotated screen with Region access permissions, which can be viewed and updated.

FIG. 72 shows a sample annotated screen for creating a new Region with access permissions.

### User Groups

User groups are the fifth access control security level of the Absolute Insight application. Every user group has a department as its parent access control entity and inherits its access control permissions. Every user group has Functional Access Controls through which various parts of application can be controlled and permissions for those Functional Access Controls can be managed.

### Example Usage

User Groups contain the following capabilities:
Lists all available user groups
View all Functional Access Controls available for user groups
View assigned permissions on each Access Control
Update permissions of any Access Control
Create new user group access control entity
Remove any user group entity

One or more user groups can be assigned to User, which is next subsequent level in the hierarchy. If more than one user group is assigned to any user, then control permissions will be aggregated for all assigned user groups.

If a user group is assigned to any user, then it will be marked as “Locked” and cannot be removed.

FIG. 73 shows a sample annotated screen with User Groups access permissions, which can be viewed and updated.

FIG. 74 shows a sample annotated screen for creating a new User Group with access permissions.

### User

User is the sixth access control security level of the Absolute Insight application.

Since every user can have one or more user groups, then all user group permissions will be aggregated in order to get final compacted Access Controls for a user.

### Example Usage

User's interface contains the following capabilities
Lists all available users
View all user's information
Update any user's information
Create new users
Remove any user

Users have the “Locked” property. By using this property, any user can be enabled or disabled. Once a user is locked, then login authentication process will never authenticate the user to enter into the Absolute Insight application.

FIG. 75 shows a sample annotated screen with User access permissions, which can be viewed and updated.

### Security Plug-In—LDAP

The Absolute Insight application can be configured to work with Ldap authentication. In order to allow Ldap user to access application, an administrator first needs to import users into the application by providing necessary information, so that Application Access Controls can be applied while logging in. FIG. 76 shows a sample annotated screen for importing users into the application.

Analyzer Operator

In certain exemplary embodiments, a special type of operator, referred to herein as the “Analyzer Operator,” allows a user to get analytics of the data by specifying metadata about the data source provided to the Analyzer Operator such as the fields to use and labels to be used for algorithm training.

With reference to FIG. 77, at 1, using the modeling screen, the user connects a data source with the Analyzer Operator via the graphical user interface, as discussed above.

At 2, the Analyzer Operator extracts the specified meta-data from the data source.

At 3, the user uses the field selector screen to define one or more label column (fields to use as training columns for the algorithms, such as, for example, a column for indicating if a doctor is fraudulent as determined by a particular algorithm), one or more ID column of the entities to be analyzed (e.g., medical claims records generally have multiple ID fields, such as a claim ID, a provider ID, a patient ID, etc.), a date field for the analysis if more than one exists (e.g., medical claims records generally have multiple date fields, such as the date service was provided to the patient, the date the claim was submitted, the date the claim was processed, etc.), the level of the data (e.g., is the data transactional or aggregate), and a subset of the columns available to be used in the analysis.

At 4, the Analyzer Operator by default applies data cleansing on fields based on the type of field that has been identified (e.g., address, zip codes, date, SSN, latitude, longitude, etc.). For example, if a social security number is provided that is less than 9 characters long, the operator will add zeros to the number so that it becomes 9 characters long.

At 5, based on the meta-data and the data, the Analyzer Operator selects the default metric to use to compare performance of models produced by the algorithms (e.g., Classification Accuracy, Logarithmic Loss, Area Under ROC Curve, Confusion Matrix, Classification Report, etc.).

At 6, based on the meta-data provided, an “Automatic Algorithm Selector” identifies algorithm(s) that can be applied to the data (e.g., unsupervised algorithms like outlier, risk, clustering or supervised algorithms like Support Vector Machines, Decision Trees, Deep Learning, etc.). The user can override the default algorithm selections.

At 7, the Analyzer Operator then prepares the data in the form required by each of the selected algorithms, the meta-data that is required by each algorithm, and the default values for each algorithm.

At 8, each algorithm selected is then executed, and parameters of the algorithm(s) or the hyperparameters (i.e., parameters from a prior execution of the algorithm) are optimized using the Bayesian Optimization algorithm, which learns from the previously run models to refine the hyperparameters of the algorithm. This looks for the optimal model using the specific algorithm used.

At 9, the metrics for each of the optimal models produced by each of the algorithms are then generated, compared and ranked to choose the best model from the multiple models automatically produced by the Analyzer Operator.

At 10, for each of the algorithms that have produced results, generic visualization metadata is prepared and the visualizations dashboards sheets for each of the selected algorithms are produced and presented to the user, e.g., High Risk Providers.

At 11, resultant visualizations are shown to end user so that the user can interact with the visualization to understand the results.

### Miscellaneous

It should be understood from the above disclosure that illustrative embodimetns of Absolute Insight provide state of the art analytic capabilities. They also may provide statistical and predictive anlaytics, as well as imply visualizations for users, and that the analytics results produced are actionable.

It should be noted that headings are used above for convenience and are not to be construed as limiting the present invention in any way.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-along hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

Although the above discussion discloses various exemplary embodiments of the invention, it should be apparent that those skilled in the art can make various modifications that will achieve some of the advantages of the invention without departing from the true scope of the invention.

What is claimed is:

1. A healthcare fraud detection system comprising:
a user interface;
a core processing system coupled to the user interface, the core processing system also coupled to a database storage; and
a data input providing healthcare data, the data input being user selectable from at least one data source, the data input being coupled to the core processing system;
wherein the core processing system comprises a set of stored pre-defined plug-and-play applications and models configured to manipulate the data, and wherein the user interface provides drag-and-drop selection and interconnection of at least one data source and at least one pre-defined plug-and-play application or model by a user to produce a healthcare fraud detection model and displays fraud analytics data produced from execution of the healthcare fraud detection model by the core processing system, wherein the core processing system saves the healthcare fraud detection model as a reusable model for further analysis including for selection and interconnection via the user interface as part of another model.

2. The healthcare fraud detection system according to claim 1, wherein the user interface is a web-browser interface.

3. The healthcare fraud detection system according to claim 1, wherein the core processing system comprises a deep learning engine configured to process the data.

4. The healthcare fraud detection system according to claim 3, wherein the deep learning engine is a machine learning engine.

5. The healthcare fraud detection system according to claim 3, wherein the deep learning engine is configured to automatically determine a set of performance metrics and a plurality of algorithms to use for the at least one data source and create therefrom an ensemble of models, where each component in the ensemble is a deep learning model focusing on a specific type of fraud.

6. The healthcare fraud detection system according to claim 1, wherein graphs and/or dashboards are reusable artifacts that are part of a template that can be integrated with data sources, filters and models to build a complete template.

7. The healthcare fraud detection system according to claim 3, wherein the deep learning engine is configured to detect medical claim fraud in real time, or substantially in real time, from a stream of medical claims.

8. The healthcare fraud detection system according to claim 1, wherein the core processing system allows the user to alter the display of the fraud analytics data.

9. The healthcare fraud detection system according to claim 1, wherein the core processing system allows sharing of the healthcare fraud detection model over a network.

10. The healthcare fraud detection system according to claim 1, wherein the set of stored pre-defined plug-and-play applications includes an analyzer operator.

11. The healthcare fraud detection system according to claim 10, wherein the analyzer operator is configured to extract meta-data from the at least one data source, perform data cleansing on a set of user-specified fields, select a set of default metrics for use in comparing performance of a plurality of fraud detection models, select a set of operators to be applied to the data, format the data for each selected operator, execute the selected operators, and determine a best model from the plurality of models based on the execution of the selected operators.

12. The healthcare fraud detection system according to claim 1, wherein the set of stored pre-defined plug-and-play applications includes at least one filter operator.

13. The healthcare fraud detection system according to claim 1, wherein the set of stored pre-defined plug-and-play applications includes at least one fraud detection operator.

14. The healthcare fraud detection system according to claim 1, wherein the set of stored pre-defined plug-and-play applications includes at least one visualization operator.

15. The healthcare fraud detection system according to claim 1, wherein the core processing system displays the at least one data source and at least one pre-defined plug-and-play application as interconnected icons on the user interface.

16. The healthcare fraud detection system according to claim 1, wherein the core processing system allows the user to associate the at least one data source and the healthcare fraud detection model as a project.

17. The healthcare fraud detection system according to claim 16, wherein the core processing system allows sharing of the project over a network.

18. The healthcare fraud detection system according to claim 1, wherein the core processing system allows the user to export results from the healthcare fraud detection model.

19. The healthcare fraud detection system according to claim 1, further comprising:
a distributed in-memory cache coupled to the core processing unit.

20. The healthcare fraud detection system according to claim 1, wherein the core processing system runs on a distributed computing cluster and utilizes a distributed file system.
